(19) United States
(12) Patent Application Publication
(10) Pub. No.: US 2022/0348674 A1
(43) Pub. Date: Nov. 3, 2022

(54) NOVEL ANTI-CD40 ANTIBODIES

(71) Applicant: Apollomics Inc., Foster City, CA (US)

(72) Inventors: Chan GAO, Redwood City, CA (US); Mu-En LIN, South San Francisco, CA (US); Qiu YU, Moraga, CA (US); Yaohuang KE, San Francisco, CA (US)

(21) Appl. No.: 17/245,345

(22) Filed: Apr. 30, 2021

(51) Int. Cl. C07K 16/28

(52) U.S. Cl. CPC C07K 16/2878; C07K 16/2827; C07K 2317/24; C07K 2317/31; C07K 2317/622

(57) ABSTRACT

The present disclosure provides anti-CD40 antibodies or antigen-binding fragments thereof, isolated polynucleotides encoding the same, pharmaceutical compositions comprising the same, and the uses thereof.

### SEQUENCE LISTING

The sequence listing that is contained in the file named “071017-8013US01_SL_ST25”, which is 136 KB (as measured in Microsoft Windows) and was created on Apr. 30, 2021, is filed herewith by electronic submission and is incorporated by reference herein.

### BACKGROUND

### 1. Field of the Invention

The present disclosure generally relates to novel anti-human CD40 antibodies.

### 2. Description of Related Art

Cluster of differentiation 40, CD40 is a type I integral membrane glycoprotein and a member of the tumor necrosis factor (TNF) receptor superfamily. CD40 is expressed on a variety of cell types, including antigen-presenting cells (APCs) such as normal and neoplastic B cells, dendritic cells (DC), monocytes and macrophages, and nonimmune cells, including epithelial cells (e.g., keratinocytes), fibroblasts (e.g., synoviocytes), smooth muscle cells and platelets. CD40 is also expressed on a wide range of tumor cells including all B-lymphomas, 30-70% of solid tumors, melanomas and carcinomas.

Function as a costimulatory protein, CD40 is required for the activation of the APCs. When binding to its ligand CD154 (CD40L) expressed on T_{H} cells, CD40 induces a variety of downstream effects on the APCs, including secretion of cytokines (e.g., IL-1, IL-6, IL-8, IL-10, IL-12, TNF-α and MIP-1α), up-regulation of costimulatory molecules (e.g., ICAM-1, LFA-3, CD80 and CD86), and APC proliferation. CD40 signaling has been found to be essential in mediating a broad variety of immune and inflammatory responses including T cell-dependent immunoglobulin class switching, memory B cell development, and germinal center formation. CD40 is overexpressed on a wide range of malignant cells, including non-Hodgkin's and Hodgkin's lymphomas, myeloma and some carcinomas including nasopharynx, bladder, cervix, kidney and ovary cancers. The roles of CD40 in the stimulation of the immune system make CD40 an attractive target for an antibody-based immunotherapy (van Mierlo G J et al., Proc Natl Acad Sci USA. (2002) 99(8): 5561 5566; French R R et al., Nat Med. (1999) 5(5):548-553).

In particular, agonist anti-CD40 antibodies have been developed for the treatment of cancer patients. Preclinical investigations showed that agonistic monoclonal anti-CD40 antibodies drive antitumor immunity, whereby CD40-activated dendritic cells (DCs) are poised to prime or activate tumor-specific T cells. CD40 activation on DCs leads to two main cellular phenotypes. First, CD40 activation leads to upregulation of major histocompatibility complex (MHC) molecules and increased expression of immunoglobulin (Ig) superfamily costimulatory molecules such as CD80 and CD86, which are the ligands for the co-stimulatory molecules CD28. Second, CD40-activated DCs elaborate an increase of critical T cell stimulatory cytokines, including Interleukin (IL)-12 p′70, which is important to skewing immune response towards Th1 polarization. Clinical trials of CD40 agonists (e.g., CP-870,893, dacetuzumab, ADC-103, Chi Lob 7/4 and APX005M) have shown signs of clinical activity in multiple indications

However, agonist anti-CD40 antibody treatment is generally associated with toxicity in the clinic, including cytokine release syndrome (CRS) and hepatoxicity associated with increased concentrations of circulating liver enzymes alanine aminotransferase (ALT), aspartate aminotransferase (AST) and glutamate dehydrogenase (GLDH). Therefore, there is a significant need for novel anti-CD40 antibodies that can effectively treat cancer with reduced adverse effects.

### BRIEF SUMMARY OF THE INVENTION

Throughout the present disclosure, the articles “a,” “an,” and “the” are used herein to refer to one or to more than one (i.e., to at least one) of the grammatical object of the article. By way of example, “an antibody” means one antibody or more than one antibody.

The present disclosure in one aspect provides novel monoclonal anti-CD40 antibodies, amino acid and nucleotide sequences thereof, and uses thereof. In certain embodiments, the anti-CD40 antibodies provided herein show low basal activity of inducing CD40 activation and only activate CD40 signaling when the antibodies are in a form that clusters CD40 on the surface of a cell. In certain embodiments, the anti-CD40 antibodies provided herein only activate CD40 in the tumor microenvironment, thus avoiding toxicity mediated by systematic activation of CD40.

In certain embodiments, the anti-CD40 antibody provided herein comprises heavy chain (HC) variable region (VH) and a light chain (LC) variable region (VL) comprising clone-paired CDR sequences as set forth in Table 1, and variants thereof wherein one or more of the CDRs has one, two or three amino acid substitutions, additions, deletions, or combination thereof.

In certain embodiments, the anti-CD40 antibody provided herein comprises the VH and VL having amino acid sequences at least 90% or at least 95% identical to the clone-paired sequences of Table 2. In certain embodiments, the anti-CD40 antibody provided herein comprises the VH and VL having amino acid sequences identical to clone-paired sequences of Table 2.

In certain embodiments, the anti-CD40 antibody provided herein comprises: (a) a heavy chain variable region comprising heavy chain complementarity determining region (HCDR)1 of amino acid sequence TYAX₁S (SEQ ID NO: 313), wherein X₁ is M or V, HCDR2 of amino acid sequence IIDX₂X₃X₄XsX₆X₇YAX₈WX₉KG (SEQ ID NO: 314), wherein each of X₂, X₃ and X₄ is independently G or S, X₅ is F or G, X₆ is A or T, X₇ is A or V, X₈ is N or S, X₉ is A or V, and HCDR3 of amino acid sequence GX₁₀TFFX₁₁L (SEQ ID NO: 315), wherein X₁₀ is A or V, and X₁₁ is G or N; and (b) a light chain variable region comprising light chain complementarity determining region (LCDR)1 of amino acid sequence QASQSISX₁₂X₁₃LX₁₄ (SEQ ID NO: 316), wherein X₁₂ is G, N or S, X₁₃ is A, V or Y, and X₁₄ is A or S, LCDR2 of amino acid sequence RAX₁₅X₁₆LX₁₇S (SEQ ID NO: 317), wherein each of X₁₅ is A or S, X₁₆ is D or T, X₁₇ is A or E, and LCDR3 of amino acid sequence QX₁₈YYYSX₁₉X₂₀GSYX₂₁YA (SEQ ID NO: 318), wherein X₁₈ is A or S, X₁₉ is G, N or S, X₂₀ is G or S, and X₂₁ is D or S.

In certain embodiments, the anti-CD40 antibody provided herein comprises (a) the heavy chain variable region comprises HCDR1 of SEQ ID NO: 31, 41 or 51, HCDR2 of SEQ ID NO: 32, 42 or 52, and HCDR3 of SEQ ID NO: 33, 43 and 53; and (b) the light chain variable region comprises LCDR1 of SEQ ID NO: 34, 44 or 54, LCDR2 of SEQ ID NO: 35, 45 or 55, and LCDR3 of SEQ ID NO: 36, 46 or 56.

In certain embodiments, the anti-CD40 antibody provided herein further comprises an immunoglobulin constant region, optionally a constant region of Ig, or optionally a constant region of human IgG.

In certain embodiments, the anti-CD40 antibody provided herein is an anti-CD40 antigen-binding fragment. In certain embodiments, the anti-CD40 antigen-binding fragment is a scFv (single chain fragment variable), a Fab fragment, a Fab′ fragment, a F(ab)₂ fragment, or a Fv fragment.

In certain embodiments, the anti-CD40 antibody provided herein is capable of specifically binding to human CD40. In certain embodiments, the anti-CD40 antibody provided herein competes with CD40L to bind to CD40.

In certain embodiments, the anti-CD40 antibody provided herein has low basal activity in activating CD40 and has enhanced activity in activating CD40 when crosslinked. In certain embodiments, the anti-CD40 antibody provided herein is capable of selectively activating CD40 in tumor microenvironment. In certain embodiments, the anti-CD40 antibody provided herein is capable of activating dendritic cells.

In certain embodiments, the anti-CD40 antibody provided herein is a murine, a rodent, a rabbit, a chimeric, a humanized or human antibody In certain embodiments, the anti-CD40 antibody provided herein is a humanized antibody which comprises a heavy chain variable region having an amino acid sequence at least about 90% identical to SEQ ID NO: 311; and a light chain variable region having an amino acid sequence at least about 90% identical to SEQ ID NO: 312.

In certain embodiments, the anti-CD40 antibody provided herein is a bispecific antibody. In certain embodiments, the bispecific anti-CD40 antibody provided herein specifically binds to an antigen enriched in tumor microenvironment. In certain embodiments, the antigen is a tumor antigen, including without limitation, alphafetoprotein (AFP), carcinoembryonic antigen (CEA), CA-125, MUC-1, epithelial tumor antigen (ETA), tyrosinase, and melanoma-associated antigen (MAGE). In some embodiments, the anti-CD40 bispecific antibody provided herein is against an immune checkpoint, including without limitation, A2AR, B7.1, B7.2, B7-H2, B7-H3, B7-H4, B7-H6, BTLA, CD48, CD160, CD244, CTLA-4, ICOS, LAG-3, LILRB1, LILRB2, LILRB4, OX40, PD-1, PD-L1, PD-L2, SIRPalpha (CD47), TIGIT, TIM-3, TIM-1, TIM-4, and VISTA.

In certain embodiments, the bispecific anti-CD40 antibody provided herein comprises an anti-CD40 scFv. In certain embodiments, the scFv has an amino acid sequence of SEQ ID NO: 319. In certain embodiments, the scFv is fused to the C-terminus of HC constant region.

In another aspect, the present disclosure provides a pharmaceutical composition comprising the anti-CD40 antibody provided herein, and a pharmaceutically acceptable carrier.

In another aspect, the present disclosure provides an isolated polynucleotide encoding the anti-CD40 antibody provided herein.

In another aspect, the present disclosure provides a vector comprising the isolated polynucleotide provided herein.

In another aspect, the present disclosure provides a host cell comprising the vector provided herein.

In another aspect, the present disclosure provides a method of expressing the anti-CD40 antibody provided herein, comprising culturing the host cell provided herein under the condition at which the vector provided herein is expressed.

In another aspect, the present disclosure provides a method of treating a disease or condition in a subject that would benefit from modulation of CD40 activity, comprising administering to the subject a therapeutically effective amount of the anti-CD40 antibody provided herein or the pharmaceutical composition provided herein. In certain embodiments, the disease or condition is a CD40 related disease or condition. In certain embodiments, the disease or condition is cancer, autoimmune disease, inflammatory disease, or infectious disease. In certain embodiments, the cancer is adrenal cancer, bone cancer, brain cancer, breast cancer, colorectal cancer, esophageal cancer, eye cancer, gastric cancer, head and neck cancer, kidney cancer, liver cancer, lung cancer, non-small cell lung cancer, bronchioloalveolar cell lung cancer, mesothelioma, head and neck cancer, squamous cell carcinoma, lymphoma, lymphocytic leukemia, melanoma, oral cancer, ovarian cancer, cervical cancer, penile cancer, prostate cancer, pancreatic cancer, skin cancer, sarcoma, testicular cancer, thyroid cancer, uterine cancer, vaginal cancer, and Hodgkin's Disease. In certain embodiments, the subject is human. In certain embodiments, the administration is via oral, nasal, intravenous, subcutaneous, sublingual, or intramuscular administration.

In another aspect, the present disclosure provides a method of modulating CD40 activity in a CD40-expressing cell, comprising exposing the CD40-expressing cell to the anti-CD40 antibody provided herein.

In another aspect, the present disclosure provides a method of detecting presence or amount of CD40 in a sample, comprising contacting the sample with the anti-CD40 antibody provided herein, and determining the presence or the amount of CD40 in the sample.

In another aspect, the present disclosure provides a method of diagnosing a CD40 related disease or condition in a subject, comprising: a) contacting a sample obtained from the subject with the anti-CD40 antibody provided herein; b) determining presence or amount of CD40 in the sample; and c) correlating the presence or the amount of CD40 to existence or status of the CD40 related disease or condition in the subject.

In another aspect, the present disclosure provides use of the anti-CD40 antibody provided herein in the manufacture of a medicament for treating a CD40 related disease or condition in a subject.

In another aspect, the present disclosure provides use of the anti-CD40 antibody provided herein in the manufacture of a diagnostic reagent for diagnosing a CD40 related disease or condition.

In another aspect, the present disclosure provides a kit comprising the anti-CD40 antibody provided herein, useful in detecting CD40.

### BRIEF DESCRIPTION OF FIGURES

The following drawings form part of the present specification and are included to further demonstrate certain aspects of the present invention. The invention may be better understood by reference to one or more of these drawings in combination with the detailed description of specific embodiments presented herein.

FIG. 1 illustrates the structure of an exemplary anti-CD40 bispecific antibody.

FIG. 2 shows that anti-CD40 antibodies CP870,893, APX005m and 3B5, but not control IgG1 bind to cell surface human CD4 on HEK-CD40 cells in a flow cytometry experiment.

FIG. 3 shows anti-CD40 antibody 3B5 competes with CD40 L and prevents CD40 activation by CD40L.

FIG. 4 shows anti-CD40 antibodies 3B5, 2E11 and 5F9 alone show low basal activity of inducing CD40 activation and activate CD40 after the anti-CD40 antibodies are crosslinked.

FIG. 5 shows that 3B5ScFv-PD-L1 bispecific antibody competes with CD40L on CD40 binding.

FIG. 6 shows that 3B5ScFv-PD-L1 bi-specific antibody dose-dependently activates Ramos-Blue cells, only in the presence of HEK-PD-L1 cells, suggesting that the crosslink mediated CD40 agonistic activity of 3B5 relies on the bispecific binding of PD-L1. Consistently, two control anti-CD40 antibodies, CP870,893 and APX005m, whose crosslink activities are independent of PD-L1 expression, showed equal activation of Ramos-Blue cells in the presence or absence of HEK-PD-L1 cells.

FIG. 7 shows that anti-CD40 antibody 3B5 did not promote differentiation of monocyte into dendritic cells nor enhance the expression of the activation markers CD80 and CD86, while the 3B5ScFv-PD-L1 bispecific antibody significantly enhanced DC differentiation and activation in the presence of PD-L1.

FIG. 8 shows that the expression level of CD80 and CD86 were enhanced on cells treated with 3B5scFv-PD-L1 bispecific antibody. At the same concentration, 3B5ScFv-PD-L1 bispecific antibody leads to stronger activation of DCs comparing to CP870,893. Solid lines indicate with PD-L1 and dot lines are without PD-L1. Red, PDL1-3B5 scFv; green, 3B5 antibody; black, CP870,893.

### DETAILED DESCRIPTION OF THE INVENTION

The following description of the disclosure is merely intended to illustrate various embodiments of the disclosure. As such, the specific modifications discussed are not to be construed as limitations on the scope of the disclosure. It will be apparent to one skilled in the art that various equivalents, changes, and modifications may be made without departing from the scope of the disclosure, and it is understood that such equivalent embodiments are to be included herein. All references cited herein, including publications, patents and patent applications are incorporated herein by reference in their entirety.

### I. Definitions

It is to be understood that both the foregoing general description and the following detailed description are exemplary and explanatory only and are not restrictive of the invention as claimed. In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise. Also, the use of the term “portion” can include part of a moiety or the entire moiety.

The term “about” as used herein when referring to a measurable value such as an amount, a temporal duration, and the like, is meant to encompass variations of up to ±10% from the specified value. Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the disclosed subject matter. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

The term “antibody” refers to an intact immunoglobulin of any isotype, or a fragment thereof that can compete with the intact antibody for specific binding to the target antigen, and includes, for instance, chimeric, humanized, fully human, and bispecific antibodies. An “antibody” is a species of an antigen binding protein. An intact antibody will generally comprise at least two full-length heavy chains and two full-length light chains, but in some instances can include fewer chains such as antibodies naturally occurring in camelids which can comprise only heavy chains. Antibodies can be derived solely from a single source, or can be “chimeric,” that is, different portions of the antibody can be derived from two different antibodies as described further below. The antigen binding proteins, antibodies, or binding fragments can be produced in hybridomas, by recombinant DNA techniques, or by enzymatic or chemical cleavage of intact antibodies. Unless otherwise indicated, the term “antibody” includes, in addition to antibodies comprising two full-length heavy chains and two full-length light chains, derivatives, variants, fragments, and muteins thereof, examples of which are described below. Furthermore, unless explicitly excluded, antibodies include monoclonal antibodies, bispecific antibodies, minibodies, domain antibodies, synthetic antibodies (sometimes referred to herein as “antibody mimetics”), chimeric antibodies, humanized antibodies, human antibodies, antibody fusions (sometimes referred to herein as “antibody conjugates”), and fragments thereof, respectively. In some embodiments, the term also encompasses peptibodies.

Naturally occurring antibody structural units typically comprise a tetramer. Each such tetramer typically is composed of two identical pairs of polypeptide chains, each pair having one full-length “light” (in certain embodiments, about 25 kDa) and one full-length “heavy” chain (in certain embodiments, about 50-70 kDa). The amino-terminal portion of each chain typically includes a variable region of about 100 to 110 or more amino acids that typically is responsible for antigen recognition. The carboxy-terminal portion of each chain typically defines a constant region that can be responsible for effector function. Human light chains are typically classified as kappa and lambda light chains. Heavy chains are typically classified as mu, delta, gamma, alpha, or epsilon, and define the antibody's isotype as IgM, IgD, IgG, IgA, and IgE, respectively. IgG has several subclasses, including, but not limited to, IgG1, IgG2, IgG3, and IgG4. IgM has subclasses including, but not limited to, IgM1 and IgM2. IgA is similarly subdivided into subclasses including, but not limited to, IgA1 and IgA2. Within full-length light and heavy chains, typically, the variable and constant regions are joined by a “J” region of about 12 or more amino acids, with the heavy chain also including a “D” region of about 10 more amino acids. See, e.g., Fundamental Immunology, Ch. 7 (Paul, W., ed., 2nd ed. Raven Press, N.Y. (1989)) (incorporated by reference in its entirety for all purposes). The variable regions of each light/heavy chain pair typically form the antigen binding site.

The term “variable region” or “variable domain” refers to a portion of the light and/or heavy chains of an antibody, typically including approximately the amino-terminal 120 to 130 amino acids in the heavy chain and about 100 to 110 amino terminal amino acids in the light chain. In certain embodiments, variable regions of different antibodies differ extensively in amino acid sequence even among antibodies of the same species. The variable region of an antibody typically determines specificity of a particular antibody for its target.

The variable regions typically exhibit the same general structure of relatively conserved framework regions (FR) joined by three hyper variable regions, also called complementarity determining regions or CDRs. The CDRs from the two chains of each pair typically are aligned by the framework regions, which can enable binding to a specific epitope. From N-terminal to C-terminal, both light and heavy chain variable regions typically comprise the domains FR1, CDR1, FR2, CDR2, FR3, CDR3 and FR4. The assignment of amino acids to each domain is typically in accordance with the definitions of Kabat Sequences of Proteins of Immunological Interest (National Institutes of Health, Bethesda, Md. (1987 and 1991)), Chothia & Lesk, J. Mol. Biol., 196:901-917 (1987) or Chothia et al., Nature, 342:878-883 (1989).

In certain embodiments, an antibody heavy chain binds to an antigen in the absence of an antibody light chain. In certain embodiments, an antibody light chain binds to an antigen in the absence of an antibody heavy chain. In certain embodiments, an antibody binding region binds to an antigen in the absence of an antibody light chain. In certain embodiments, an antibody binding region binds to an antigen in the absence of an antibody heavy chain. In certain embodiments, an individual variable region specifically binds to an antigen in the absence of other variable regions.

In certain embodiments, definitive delineation of a CDR and identification of residues comprising the binding site of an antibody is accomplished by solving the structure of the antibody and/or solving the structure of the antibody-ligand complex. In certain embodiments, that can be accomplished by any of a variety of techniques known to those skilled in the art, such as X-ray crystallography. In certain embodiments, various methods of analysis can be employed to identify or approximate the CDR regions. Examples of such methods include, but are not limited to, the Kabat definition, the Chothia definition, the AbM definition and the contact definition.

The Kabat definition is a standard for numbering the residues in an antibody and is typically used to identify CDR regions. See, e.g., Johnson & Wu, Nucleic Acids Res., 28: 214-8 (2000). The Chothia definition is similar to the Kabat definition, but the Chothia definition takes into account positions of certain structural loop regions. See, e.g., Chothia et al., J. Mol. Biol., 196: 901-17 (1986); Chothia et al., Nature, 342: 877-83 (1989). The AbM definition uses an integrated suite of computer programs produced by Oxford Molecular Group that model antibody structure. See, e.g., Martin et al., Proc Natl Acad Sci (USA), 86:9268-9272 (1989); “AbM™, A Computer Program for Modeling Variable Regions of Antibodies,” Oxford, UK; Oxford Molecular, Ltd. The AbM definition models the tertiary structure of an antibody from primary sequence using a combination of knowledge databases and ab initio methods, such as those described by Samudrala et al., “Ab Initio Protein Structure Prediction Using a Combined Hierarchical Approach,” in PROTEINS, Structure, Function and Genetics Suppl., 3:194-198 (1999). The contact definition is based on an analysis of the available complex crystal structures. See, e.g., MacCallum et al., J. Mol. Biol., 5:732-45 (1996).

By convention, the CDR regions in the heavy chain are typically referred to as HCDR1, HCDR2, and HCDR3 and are numbered sequentially in the direction from the amino terminus to the carboxy terminus. The CDR regions in the light chain are typically referred to as LCDR1, LCDR2, and LCDR3 and are numbered sequentially in the direction from the amino terminus to the carboxy terminus.

The term “light chain” includes a full-length light chain and fragments thereof having sufficient variable region sequence to confer binding specificity. A full-length light chain includes a variable region domain, VL, and a constant region domain, CL. The variable region domain of the light chain is at the amino-terminus of the polypeptide. Light chains include kappa chains and lambda chains.

The term “heavy chain” includes a full-length heavy chain and fragments thereof having sufficient variable region sequence to confer binding specificity. A full-length heavy chain includes a variable region domain, VH, and three constant region domains, CH1, CH2, and CH3. The VH domain is at the amino-terminus of the polypeptide, and the CH domains are at the carboxyl-terminus, with the CH3 being closest to the carboxy-terminus of the polypeptide. Heavy chains can be of any isotype, including IgG (including IgG1, IgG2, IgG3 and IgG4 subtypes), IgA (including IgA1 and IgA2 subtypes), IgM and IgE.

The term “antigen” refers to a substance capable of inducing adaptive immune responses. Specifically, an antigen is a substance specifically bound by antibodies or T lymphocyte antigen receptors. Antigens are usually proteins and polysaccharides, less frequently also lipids. Suitable antigens include without limitation parts of bacteria (coats, capsules, cell walls, flagella, fimbrai, and toxins), viruses, and other microorganisms. Antigens also include tumor antigens, e.g., antigens generated by mutations in tumors. As used herein, antigens also include immunogens and haptens.

The term “antigen-binding fragment” as used herein refers to an antibody fragment formed from a portion of an antibody comprising one or more CDRs, or any other antibody fragment that binds to an antigen but does not comprise an intact native antibody structure. Examples of antigen-binding fragment include, without limitation, a diabody, a Fab, a Fab′, a F(ab′)₂, an Fv fragment, a disulfide stabilized Fv fragment (dsFv), a (dsFv)₂, a bispecific dsFv (dsFv-dsFv′), a disulfide stabilized diabody (ds diabody), a single-chain fragment variable (scFv), an scFv dimer (bivalent diabody), a camelized single domain antibody, a nanobody, a domain antibody, and a bivalent domain antibody. An antigen-binding fragment is capable of binding to the same antigen to which the parent antibody binds.

A “Fab fragment” comprises one light chain and the C_{H}1 and variable domains of one heavy chain. The heavy chain of a Fab molecule cannot form a disulfide bond with another heavy chain molecule.

A “Fab′ fragment” comprises one light chain and a portion of one heavy chain that contains the VH domain and the C_{H}1 domain and also the region between the C_{H}1 and C_{H}2 domains, such that an interchain disulfide bond can be formed between the two heavy chains of two Fab′ fragments to form an F(ab′)₂ molecule.

A “F(ab′)₂ fragment” contains two light chains and two heavy chains containing a portion of the constant region between the C_{H}1 and C_{H}2 domains, such that an interchain disulfide bond is formed between the two heavy chains. A F(ab′)₂ fragment thus is composed of two Fab′ fragments that are held together by a disulfide bond between the two heavy chains.

“Fv” with regard to an antibody refers to the smallest fragment of the antibody to bear the complete antigen-binding site. An Fv fragment consists of the variable domain of a single light chain bound to the variable domain of a single heavy chain.

A “dsFv” refers to a disulfide-stabilized Fv fragment that the linkage between the variable domain of a single light chain and the variable domain of a single heavy chain is a disulfide bond. In some embodiments, a “(dsFv)₂” or “(dsFv-dsFv′)” comprises three peptide chains: two VH moieties linked by a peptide linker (e.g., a long flexible linker) and bound to two VL moieties, respectively, via disulfide bridges. In some embodiments, dsFv-dsFv′ is bispecific in which each disulfide paired heavy and light chain has a different antigen specificity.

“Single-chain Fv antibody” or “scFv” refers to an engineered antibody consisting of a light chain variable domain and a heavy chain variable domain connected to one another directly or via a peptide linker sequence (Huston J S et al. Proc Natl Acad Sci USA, 85:5879(1988)).

An “Fc” region comprises two heavy chain fragments comprising the C_{H}2 and C_{H}3 domains of an antibody. The two heavy chain fragments are held together by two or more disulfide bonds and by hydrophobic interactions of the C_{H}3 domains. The Fc region of the antibody is responsible for various effector functions such as antibody-dependent cell-mediated cytotoxicity (ADCC), and complement dependent cytotoxicity (CDC), but does not function in antigen binding.

“Single-chain Fv-Fc antibody” or “scFv-Fc” refers to an engineered antibody consisting of a scFv connected to the Fc region of an antibody.

“Camelized single domain antibody,” “heavy chain antibody,” or “HCAb” refers to an antibody that contains two V_{H} domains and no light chains (Riechmann L. and Muyldermans S., J Immunol Methods. December 10; 231(1-2):25-38 (1999); Muyldermans S., J Biotechnol. June; 74(4):277-302 (2001); WO94/04678; WO94/25591; U.S. Pat. No. 6,005,079). Heavy chain antibodies were originally derived from Camelidae (camels, dromedaries, and llamas). Although devoid of light chains, camelized antibodies have an authentic antigen-binding repertoire (Hamers-Casterman C. et al., Nature. June 3; 363(6428):446-8 (1993); Nguyen V K. et al. “Heavy-chain antibodies in Camelidae; a case of evolutionary innovation,” Immunogenetics. April; 54(1):39-47 (2002); Nguyen V K. et al. Immunology. May; 109(1):93-101 (2003)). The variable domain of a heavy chain antibody (VHH domain) represents the smallest known antigen-binding unit generated by adaptive immune responses (Koch-Nolte F. et al., FASEB J. November; 21(13):3490-8. Epub 2007 Jun. 15 (2007)).

A “nanobody” refers to an antibody fragment that consists of a VHH domain from a heavy chain antibody and two constant domains, C_{H}2 and C_{H}3.

“Diabodies” or “dAbs” include small antibody fragments with two antigen-binding sites, wherein the fragments comprise a VH domain connected to a V_{L} domain in the same polypeptide chain (V_{H}-V_{L} or V_{L}-V_{H}) (see, e.g., Holliger P. et al., Proc Natl Acad Sci USA. July 15; 90(14):6444-8 (1993); EP404097; WO93/11161). By using a linker that is too short to allow pairing between the two domains on the same chain, the domains are forced to pair with the complementary domains of another chain, thereby creating two antigen-binding sites. The antigen-binding sites may target the same or different antigens (or epitopes). In certain embodiments, a “bispecific ds diabody” is a diabody target two different antigens (or epitopes). In certain embodiments, an “scFv dimer” is a bivalent diabody or bivalent ScFv (BsFv) comprising V_{H}-V_{L} (linked by a peptide linker) dimerized with another V_{H}-V_{L} moiety such that VH's of one moiety coordinate with the VL's of the other moiety and form two binding sites which can target the same antigens (or epitopes) or different antigens (or epitopes). In other embodiments, an “scFv dimer” is a bispecific diabody comprising V_{H1}-V_{L2} (linked by a peptide linker) associated with V_{L1}-V_{H2} (also linked by a peptide linker) such that V_{H1} and V_{L1} coordinate and V_{H2} and V_{L2} coordinate and each coordinated pair has a different antigen specificity.

A “domain antibody” refers to an antibody fragment containing only the variable domain of a heavy chain or the variable domain of a light chain. In certain instances, two or more VH domains are covalently joined with a peptide linker to create a bivalent or multivalent domain antibody. The two VH domains of a bivalent domain antibody may target the same or different antigens.

As used herein, a “bispecific antibody” refers to an artificial antibody which has fragments derived from two different monoclonal antibodies and is capable of binding to two different epitopes. The two epitopes may present on the same antigen, or they may present on two different antigens.

The term “chimeric” as used herein, means an antibody or antigen-binding fragment, having a portion of heavy and/or light chain derived from one species, and the rest of the heavy and/or light chain derived from a different species. In an illustrative example, a chimeric antibody may comprise a constant region derived from human and a variable region from a non-human animal, such as from mouse or rabbit. In some embodiments, the non-human animal is a mammal, for example, a mouse, a rat, a rabbit, a goat, a sheep, a guinea pig, or a hamster.

The term “humanized” as used herein means that the antibody or antigen-binding fragment comprises CDRs derived from non-human animals, FR regions derived from human, and when applicable, the constant regions derived from human.

“CD40” as used herein, refers to CD40 derived from any vertebrate source, including mammals such as primates (e.g. humans, monkeys) and rodents (e.g., mice and rats). Exemplary sequence of human CD40 includes human CD40 protein (NCBI Ref Seq No. ALQ33424.1). Exemplary sequence of CD40 includes mouse CD40 protein (NCBI Ref Seq No. AAB08705.1); Rattus norvegicus (Rat) CD40 protein (NCBI Ref Seq No. AAH97949.1). The term “CD40” as used herein is intended to encompass any form of CD40, for example, 1) native unprocessed CD40 molecule, “full-length” CD40 chain or naturally occurring variants of CD40, including, for example, splice variants or allelic variants; 2) any form of CD40 that results from processing in the cell; or 3) full length, a fragment (e.g., a truncated form, an extracellular/transmembrane domain) or a modified form (e.g. a mutated form, a glycosylated/PEGylated, a His-tag/immunofluorescence fused form) of CD40 subunit generated through recombinant method.

The term “anti-CD40 antibody” refers to an antibody that is capable of specifically binding to CD40 (e.g. human or mouse or rabbit CD40).

The term “specific binding” or “specifically binds” as used herein refers to a non-random binding reaction between two molecules, such as for example between an antibody and an antigen. In certain embodiments, the antibodies or antigen-binding fragments provided herein specifically bind to human and/or CD40 with a binding affinity (K_{D}) of ≤10⁻⁶ M (e.g., ≤5×10⁻⁷ M, ≤2×10⁻⁷ M, ≤10⁻⁷ M, ≤5×10⁻⁸ M, ≤2×10⁻⁸ M, ≤10⁻⁸M, ≤5×10⁻⁹M, ≤4×10⁻⁹M, ≤3×10⁻⁹M, ≤2×10⁻⁹ M, or ≤10⁻⁹ M). K_{D} used herein refers to the ratio of the dissociation rate to the association rate (k_{off}/kₒₙ), which may be determined by using any conventional method known in the art, including but are not limited to surface plasmon resonance method, microscale thermophoresis method, HPLC-MS method and flow cytometry (such as FACS) method. In certain embodiments, the K_{D} value can be appropriately determined by using flow cytometry.

The ability to “block binding” or “compete with” as used herein refers to the ability of an antibody to inhibit the binding interaction between two molecules (e.g. human CD40 and an CD40L) to any detectable degree. In certain embodiments, an antibody or antigen-binding fragment that blocks binding between two molecules inhibits the binding interaction between the two molecules by at least 85%, or at least 90%. In certain embodiments, this inhibition may be greater than 85%, or greater than 90%.

The term “epitope” as used herein refers to the specific group of atoms or amino acids on an antigen to which an antibody binds. Two antibodies may bind the same or a closely related epitope within an antigen if they exhibit competitive binding for the antigen. For example, if an antibody or antigen-binding fragment blocks binding of a reference antibody to the antigen by at least 85%, or at least 90%, or at least 95%, then the antibody or antigen-binding fragment may be considered to bind the same/closely related epitope as the reference antibody.

Those skilled in the art will recognize that it is possible to determine, without undue experimentation, if a given antibody binds to the same epitope as the antibody of present disclosure by ascertaining whether the former prevents the latter from binding to a CD40 antigen polypeptide. If the given antibody competes with the antibody of present disclosure, as shown by a decrease in binding by the antibody of present disclosure to the CD40 antigen polypeptide, then the two antibodies bind to the same, or a closely related, epitope. Or if the binding of a given antibody to the CD40 antigen polypeptide was inhibited by the antibody of present disclosure, then the two antibodies bind to the same, or a closely related, epitope.

A “conservative substitution” with reference to amino acid sequence refers to replacing an amino acid residue with a different amino acid residue having a side chain with similar physiochemical properties. For example, conservative substitutions can be made among amino acid residues with hydrophobic side chains (e.g. Met, Ala, Val, Leu, and Ile), among residues with neutral hydrophilic side chains (e.g. Cys, Ser, Thr, Asn and Gln), among residues with acidic side chains (e.g. Asp, Glu), among amino acids with basic side chains (e.g. His, Lys, and Arg), or among residues with aromatic side chains (e.g. Trp, Tyr, and Phe). As known in the art, conservative substitution usually does not cause significant change in the protein conformational structure, and therefore could retain the biological activity of a protein.

“Effector functions” as used herein refer to biological activities attributable to the binding of Fc region of an antibody to its effectors such as C1 complex and Fc receptor. Exemplary effector functions include: complement dependent cytotoxicity (CDC) induced by interaction of antibodies and C1q on the C1 complex; antibody-dependent cell-mediated cytotoxicity (ADCC) induced by binding of Fc region of an antibody to Fc receptor on an effector cell; and phagocytosis.

The term “homologue” and “homologous” as used herein are interchangeable and refer to nucleic acid sequences (or its complementary strand) or amino acid sequences that have sequence identity of at least 80% (e.g., at least 85%, 88%, 90%, 91%, 92%, 93%, 94%, 95%, 96%, 97%, 98%, 99%) to another sequences when optimally aligned.

The term “host cell” means a cell that has been transformed, or is capable of being transformed, with a nucleic acid sequence and thereby expresses a gene of interest. The term includes the progeny of the parent cell, whether or not the progeny is identical in morphology or in genetic make-up to the original parent cell, so long as the gene of interest is present.

An “isolated” substance has been altered by the hand of man from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide is “isolated” if it has been sufficiently separated from the coexisting materials of its natural state so as to exist in a substantially pure state. An “isolated nucleic acid sequence” refers to the sequence of an isolated nucleic acid molecule. In certain embodiments, an “isolated antibody or antigen-binding fragment thereof” refers to the antibody or antigen-binding fragments having a purity of at least 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99% as determined by electrophoretic methods (such as SDS-PAGE, isoelectric focusing, capillary electrophoresis), or chromatographic methods (such as ion exchange chromatography or reverse phase HPLC).

“Percent (%) sequence identity” with respect to amino acid sequence (or nucleic acid sequence) is defined as the percentage of amino acid (or nucleic acid) residues in a candidate sequence that are identical to the amino acid (or nucleic acid) residues in a reference sequence, after aligning the sequences and, if necessary, introducing gaps, to achieve the maximum number of identical amino acids (or nucleic acids). Conservative substitution of the amino acid residues may or may not be considered as identical residues. Alignment for purposes of determining percent amino acid (or nucleic acid) sequence identity can be achieved, for example, using publicly available tools such as BLASTN, BLASTp (available on the website of U.S. National Center for Biotechnology Information (NCBI), see also, Altschul S. F. et al, J. Mol. Biol., 215:403-410 (1990); Stephen F. et al, Nucleic Acids Res., 25:3389-3402 (1997)), ClustalW2 (available on the website of European Bioinformatics Institute, see also, Higgins D. G. et al, Methods in Enzymology, 266:383-402 (1996); Larkin M. A. et al, Bioinformatics (Oxford, England), 23(21): 2947-8 (2007)), and ALIGN or Megalign (DNASTAR) software. Those skilled in the art may use the default parameters provided by the tool, or may customize the parameters as appropriate for the alignment, such as for example, by selecting a suitable algorithm.

The pharmaceutically acceptable carriers useful in this invention are conventional. Remington's Pharmaceutical Sciences, by E. W. Martin, Mack Publishing Co., Easton, Pa., 15th Edition (1975), describes compositions and formulations suitable for pharmaceutical delivery of the fusion proteins herein disclosed. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

The term “therapeutically effective amount” or “effective dosage” as used herein refers to the dosage or concentration of a drug effective to treat a disease or condition. For example, with regard to the use of the monoclonal antibodies or antigen-binding fragments thereof disclosed herein to treat cancer, a therapeutically effective amount is the dosage or concentration of the monoclonal antibody or antigen-binding fragment thereof capable of reducing the tumor volume, eradicating all or part of a tumor, inhibiting or slowing tumor growth or cancer cell infiltration into other organs, inhibiting growth or proliferation of cells mediating a cancerous condition, inhibiting or slowing tumor cell metastasis, ameliorating any symptom or marker associated with a tumor or cancerous condition, preventing or delaying the development of a tumor or cancerous condition, or some combination thereof.

“Treating” or “treatment” of a condition as used herein includes preventing or alleviating a condition, slowing the onset or rate of development of a condition, reducing the risk of developing a condition, preventing or delaying the development of symptoms associated with a condition, reducing or ending symptoms associated with a condition, generating a complete or partial regression of a condition, curing a condition, or some combination thereof.

The term “vector” as used herein refers to a vehicle into which a polynucleotide encoding a protein may be operably inserted so as to bring about the expression of that protein. A vector may be used to transform, transduce, or transfect a host cell so as to bring about expression of the genetic element it carries within the host cell. Examples of vectors include plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. Categories of animal viruses used as vectors include retrovirus (including lentivirus), adenovirus, adeno-associated virus, herpesvirus (e.g., herpes simplex virus), poxvirus, baculovirus, papillomavirus, and papovavirus (e.g., SV40). A vector may contain a variety of elements for controlling expression, including promoter sequences, transcription initiation sequences, enhancer sequences, selectable elements, and reporter genes. In addition, the vector may contain an origin of replication. A vector may also include materials to aid in its entry into the cell, including but not limited to a viral particle, a liposome, or a protein coating. A vector can be an expression vector or a cloning vector. The present disclosure provides vectors (e.g., expression vectors) containing the nucleic acid sequence provided herein encoding the antibody or antigen-binding fragment thereof, at least one promoter (e.g., SV40, CMV, EF-1α) operably linked to the nucleic acid sequence, and at least one selection marker. Examples of vectors include, but are not limited to, retrovirus (including lentivirus), adenovirus, adeno-associated virus, herpesvirus (e.g., herpes simplex virus), poxvirus, baculovirus, papillomavirus, papovavirus (e.g., SV40), lambda phage, and M13 phage, plasmid pcDNA3.3, pMD18-T, pOptivec, pCMV, pEGFP, pIRES, pQD-Hyg-GSeu, pALTER, pBAD, pcDNA, pCal, pL, pET, pGEMEX, pGEX, pCI, pEGFT, pSV2, pFUSE, pVITRO, pVIVO, pMAL, pMONO, pSELECT, pUNO, pDUO, Psg5L, pBABE, pWPXL, pBI, p15TV-L, pPro18, pTD, pRS10, pLexA, pACT2.2, pCMV-SCRIPT®, pCDM8, pCDNA1.1/amp, pcDNA3.1, pRc/RSV, PCR 2.1, pEF-1, pFB, pSG5, pXT1, pCDEF3, pSVSPORT, pEF-Bos etc.

### II. Anti-CD40 Antibody

The present disclosure in one aspect provides anti-CD40 antibodies. In certain embodiments, the anti-CD40 antibodies provided herein show low basal activity of inducing CD40 activation and only activate CD40 signaling when the antibodies are in the form that clusters CD40 on the surface of a cell. In certain embodiments, the anti-CD40 antibodies provided herein activate the CD40 signaling when crosslinked by Fab2 fragment targeting IgG Fc. In certain embodiments, the anti-CD40 antibodies provided herein activate the CD40 signaling when in a form of bispecific antibody.

In certain embodiments, the anti-CD40 antibodies provided herein only activate CD40 in the tumor microenvironment, thus avoiding toxicity mediated by systematic activation of CD40.

In certain embodiments, binding of the anti-CD40 antibody provided herein to CD40 on dendritic cells induces DC maturation as manifested by increasing expression of co-stimulatory molecules such as CD80, CD83, CD86. These lead to potent T cell responses (see Stout, R. D., J. Suttles, Immunol (1996) Today 17:487-492; Brendan O'Sullivan, Ranjeny Thomas, Critical Reviews in Immunology (2003) 23: 83-107; Cella, M., D. Scheidegger, K. Palmer-Lehmann, P. Lane, A. Lanzavecchia, G. Alber, J Exp Med (1996) 184:747-452). In certain embodiments, binding of the anti-CD40 antibody provided herein to CD40 induces DC activation measured as upregulation of co-stimulatory molecules such as CD80, CD83, CD86. In certain embodiments, binding of the anti-CD40 antibody provided herein to CD40 induces B cell activation measured as upregulation of co-stimulatory molecules such as B7 family (CD80, CD86).

In certain embodiments, the anti-CD40 antibodies provided herein competes with CD40 ligands for binding to CD40. Naturally occurring ligand of CD40 is CD40L (also referred to as CD154, gp39, and TRAP), a TNF superfamily member. CD40L is a transmembrane protein expressed predominantly on activated CD4⁺ T cells and a small subset of CD8⁺ T cells (Reviewed by Van Kooten C. and Banchereau, J Leukoc Biol (2000) 67(1):2-17). CD40L exists on such cells as a trimeric structure, which induces oligomerization of its receptor upon binding.

The interaction of CD40 with CD40L induces both humoral and cell-mediated immune responses. CD40 regulates this ligand-receptor pair to activate B cells and other antigen-presenting cells (APC) including dendritic cells (DCs) (see, Toubi and Shoenfeld, Autoimmunity (2004) 37:457-64; Kiener, et al., J Immunol (1995) 155(10):4917-25). Activation of CD40 on B cells induces proliferation, immunoglobulin class switching, antibody secretion, and also has a role in the development of germinal centers and the survival of memory B cells, all of which are essential to humoral immune responses (Kehry M R. J Immunol (1996) 156: 2345-2348). Binding of CD40L to CD40 on dendritic cells induces DC maturation as manifested by increasing expression of co-stimulatory molecules such as B7 family (CD80, CD86) and production of proinflammatory cytokines such as interleukin 12. These lead to potent T cell responses (Stout, R. D., J. Suttles, Immunol (1996) Today 17:487-492; Brendan O'Sullivan, Ranjeny Thomas, Critical Reviews in Immunology (2003) 23: 83-107; Cella, M., et al., Exp Med (1996) 184:747-452). As CD40-CD40L plays a crucial role in driving an efficient T cell-dependent immune response, by competing with CD40L for binding to CD40, the anti-CD40 antibodies provided herein may block the CD40 signaling via CD40L, and thereby suppressing a pathogenic autoimmune response.

In certain embodiments, the anti-CD40 antibodies provided herein comprises heavy chain (HC) variable region (VH) and a light chain (LC) variable region (VL) comprising clone-paired CDR sequences as set forth in Table 1, and variants thereof wherein one or more of the CDRs has one, two or three amino acid substitution, additions, deletions, or combination thereof.

TABLE 1
Clone-Paired CDR Sequences

| Clone | Chain | CDR1 | CDR2 | CDR3 |
|---|---|---|---|---|
| 1D3 | H1 | SEQ ID NO: 1 | SEQ ID NO: 2 | SEQ ID NO: 3 |
| | | NYHMC | YNSYGGSAYYASWAKG | AYLDYNGDMGFAL |
| | L1 | SEQ ID NO: 4 | SEQ ID NO: 5 | SEQ ID NO: 6 |
| | | QASQSISSYLS | RASTLES | QTNYYDSGNTYGT |
| 2B8 | H1 | SEQ ID NO: 11 | SEQ ID NO: 12 | SEQ ID NO: 13 |
| | | SYAMG | VIASNSGISYYATWAEG | GYIGDGAL |
| | L1 | SEQ ID NO: 14 | SEQ ID NO: 15 | SEQ ID NO: 16 |
| | | QSSKSVYYNNWLA | RASTLAS | AGGYSSDSDTT |
| 3B12 | H1 | SEQ ID NO: 21 | SEQ ID NO: 22 | SEQ ID NO: 23 |
| | | SYAMG | VIASNSGISYYATWAEG | GYIGDGAL |
| | L2 | SEQ ID NO: 24 | SEQ ID NO: 25 | SEQ ID NO: 26 |
| | | QSSKTVYYNNWLA | RASTLAS | AGGFSSDSDTT |
| 3B5 | H1 | SEQ ID NO: 31 | SEQ ID NO: 32 | SEQ ID NO: 33 |
| | | TYAVS | IIDSGGGTAYASWVKG | GVTFFGL |
| | L1 | SEQ ID NO: 34 | SEQ ID NO: 35 | SEQ ID NO: 36 |
| | | QASQSISNYLS | RASDLAS | QAYYYSGGGSYDYA |
| 2E3 | H2 | SEQ ID NO: 41 | SEQ ID NO: 42 | SEQ ID NO: 43 |
| | | TYAMS | IIDGSSFTVYANWAKG | GATFFGL |
| | L1 | SEQ ID NO: 44 | SEQ ID NO: 45 | SEQ ID NO: 46 |
| | | QASQSISGVLA | RAADLAS | QSYYYSNSGSYSYA |
| 4A7 | H1 | SEQ ID NO: 51 | SEQ ID NO: 52 | SEQ ID NO: 53 |
| | | TYAMS | IIDGGGFAVYANWAKG | GVTFFNL |
| | LCDR | SEQ ID NO: 54 | SEQ ID NO: 55 | SEQ ID NO: 56 |
| | | QASQSISSALA | RASTLES | QSYYYSSGGSYDYA |
| 3E12 | H2 | SEQ ID NO: 61 | SEQ ID NO: 62 | SEQ ID NO: 63 |
| | | NYRMI | VIDRNDNTYYATWAKS | DTDYTTGGI |
| | L1 | SEQ ID NO: 64 | SEQ ID NO: 65 | SEQ ID NO: 66 |
| | | QASQNIGDNLA | GASTLAS | QSNYYSTSSSYYNS |
| 4B7 | H2 | SEQ ID NO: 71 | SEQ ID NO: 72 | SEQ ID NO: 73 |
| | | NYRVI | GIDRSAVAYYASWAKS | DTDYTSGGI |
| | L2 | SEQ ID NO: 74 | SEQ ID NO: 75 | SEQ ID NO: 76 |
| | | QASQNIGGNLA | GSSTLES | QSNYWSRSSSYVNS |
| 4F11 | H2 | SEQ ID NO: 81 | SEQ ID NO: 82 | SEQ ID NO: 83 |
| | | SNALS | IINSYGSTYYASWAKG | VLEYSSTDYGDWGYFNL |
| | L2 | SEQ ID NO: 84 | SEQ ID NO: 85 | SEQ ID NO: 86 |
| | | QASEDIASNLA | GASNLES | QTSYYDSNTRNV |
| 6C2 | H1 | SEQ ID NO: 91 | SEQ ID NO: 92 | SEQ ID NO: 93 |
| | | TYYMI | IISSNGNAYYATWAKG | AASIANSYYFNV |
| | L2 | SEQ ID NO: 94 | SEQ ID NO: 95 | SEQ ID NO: 96 |
| | | QSSQSVRDNNWLS | GASNLES | QGGFSTYSDNS |
| 6E1 | H2 | SEQ ID NO: 101 | SEQ ID NO: 102 | SEQ ID NO: 103 |
| | | SYAMT | IIDTIGSTYYASWAKG | DYIGSTSGKKL |
| | L2 | SEQ ID NO: 104 | SEQ ID NO: 105 | SEQ ID NO: 106 |
| | | QSSKSVYYKNWLA | GASTLAS | AGGYSSDSDTG |
| 6G8 | H2 | SEQ ID NO: 111 | SEQ ID NO: 112 | SEQ ID NO: 113 |
| | | TYYMT | YINTGGSTYYASRAKG | ADASEHNYNGLNI |
| | L1 | SEQ ID NO: 114 | SEQ ID NO: 115 | SEQ ID NO: 116 |
| | | QASESISSNLA | RASTLAS | QSNYGSISSSYGNA |
| 6H9 | H2 | SEQ ID NO: 121 | SEQ ID NO: 122 | SEQ ID NO: 123 |
| | | SYAMG | VIASNSGISYYATWAEG | GYIGDGAL |
| | L2 | SEQ ID NO: 124 | SEQ ID NO: 125 | SEQ ID NO: 126 |
| | | QSSKTVYYNNWLA | RASTLAS | AGGFSSDSDTT |
| 7A6 | H2 | SEQ ID NO: 131 | SEQ ID NO: 132 | SEQ ID NO: 133 |
| | | TWTMS | IINSDSNTYYASWAKG | GVGGYSSGPDYLDI |
| | L2 | SEQ ID NO: 134 | SEQ ID NO: 135 | SEQ ID NO: 136 |
| | | QASQSIYRSLA | HASDLAS | QSYYYSGINTYGNA |
| 7C11 | H2 | SEQ ID NO: 141 | SEQ ID NO: 142 | SEQ ID NO: 143 |
| | | FNGMG | FIRVTAGTDYANWAKG | GHANNDAYVYFNL |
| | L2 | SEQ ID NO: 144 | SEQ ID NO: 145 | SEQ ID NO: 146 |
| | | QASQSIDRYLA | YTSKLAS | QSYFTSDDANS |
| 8C10 | H1 | SEQ ID NO: 151 | SEQ ID NO: 152 | SEQ ID NO: 153 |
| | | TDGMG | TIRVTGGTDFATWAKG | GHANNDAYVYFNL |
| | L1 | SEQ ID NO: 154 | SEQ ID NO: 155 | SEQ ID NO: 156 |
| | | QASQSIDRYLA | AAANLAS | QSYFTSDDGNT |
| 2C10 | H2 | SEQ ID NO: 161 | SEQ ID NO: 162 | SEQ ID NO: 163 |
| | | RYRMS | TINNDAGASYASWVNG | AGDNIYVDLYNL |
| | L4 | SEQ ID NO: 164 | SEQ ID NO: 165 | SEQ ID NO: 166 |
| | | QASENIFSYLA | GASNLAS | QFTNYGSSDIRSP |
| 7G6 | H2 | SEQ ID NO: 171 | SEQ ID NO: 172 | SEQ ID NO: 173 |
| | | TNGMG | TIRVTGGTDYATWAKG | GHANNDAYVYFNL |
| | L2 | SEQ ID NO: 174 | SEQ ID NO: 175 | SEQ ID NO: 176 |
| | | QASQSIDRYLA | YTSTLTS | QSYFTSDDANT |
| 7H11 | H2 | SEQ ID NO: 181 | SEQ ID NO: 182 | SEQ ID NO: 183 |
| | | KYWMA | ILSTTGSTYYASWAKG | GNYIGNGYTWDI |
| | L2 | SEQ ID NO: 184 | SEQ ID NO: 185 | SEQ ID NO: 186 |
| | | QASQSIGSYLA | SPSTLAS | QSTAVGIT |
| 8C6 | H1 | SEQ ID NO: 191 | SEQ ID NO: 192 | SEQ ID NO: 193 |
| | | SYYII | IIDTGGSADYASWAKG | ADVGSVGSI |
| | L2 | SEQ ID NO: 194 | SEQ ID NO: 195 | SEQ ID NO: 196 |
| | | QASQNIYTNLA | YASTLAS | QCTYIKWRHVCG |
| 7D2 | H2 | SEQ ID NO: 201 | SEQ ID NO: 202 | SEQ ID NO: 203 |
| | | NYYMG | WITSGRIAAYANWAKG | TTTSDAYFFPRLGL |
| | L1 | SEQ ID NO: 204 | SEQ ID NO: 205 | SEQ ID NO: 206 |
| | | QSSQSVDNNRLS | DASTLAS | AGGYSFPSENA |
| 7F10 | H1 | SEQ ID NO: 211 | SEQ ID NO: 212 | SEQ ID NO: 213 |
| | | DYYMT | IISSSGNTYNATWANG | EDGSGGRFDAFDL |
| | L1 | SEQ ID NO: 214 | SEQ ID NO: 215 | SEQ ID NO: 216 |
| | | QASQSISSYCS | RASILQS | QQGSTIANIENT |
| 2E11 | H1 | SEQ ID NO: 221 | SEQ ID NO: 222 | SEQ ID NO: 223 |
| | | RYAMG | IITSNSGDTYYATWAKG | GYIGAPNL |
| | L2 | SEQ ID NO: 224 | SEQ ID NO: 225 | SEQ ID NO: 226 |
| | | QSSKTVYYNNYLS | YASTLSS | AGGYNSDSDNT |
| 5F9 | H2 | SEQ ID NO: 231 | SEQ ID NO: 232 | SEQ ID NO: 233 |
| | | GYVMG | WISFGGDPYYASWVKG | STYGDI |
| | L2 | SEQ ID NO: 234 | SEQ ID NO: 235 | SEQ ID NO: 236 |
| | | QASQSISSRLA | SASTLAS | QNHGYATSESYTNT |
| 5D10 | H10 | SEQ ID NO: 241 | SEQ ID NO: 242 | SEQ ID NO: 243 |
| | | DHTMI | FINIGGRAYYASWAKG | GDTHGYSYPEI |
| | L9 | SEQ ID NO: 244 | SEQ ID NO: 245 | SEQ ID NO: 246 |
| | | QASQTIYSYLS | RASTLAS | QNYYDSTSSNA |
| 192 | H2 | SEQ ID NO: 251 | SEQ ID NO: 252 | SEQ ID NO: 253 |
| | | SYAMN | MIATGGKTFYAGWAEG | GYVGDIYFGF |
| | L2 | SEQ ID NO: 254 | SEQ ID NO: 255 | SEQ ID NO: 256 |
| | | QSSKSVYYNTHLS | RASTLAS | AGGYANDVDNG |
| 194 | H2 | SEQ ID NO: 261 | SEQ ID NO: 262 | SEQ ID NO: 263 |
| | | SYAMS | VMNTGGSAHYATWAKG | GYIGSPNL |
| | L1 | SEQ ID NO: 264 | SEQ ID NO: 265 | SEQ ID NO: 266 |
| | | QSSKSVYYNNYLS | LASTLAS | AGGYTDSDDA |
| 195 | H2 | SEQ ID NO: 271 | SEQ ID NO: 272 | SEQ ID NO: 273 |
| | | SYAMN | MIATGGKTFYAGWAEG | GYVGDIYFGF |
| | L1 | SEQ ID NO: 274 | SEQ ID NO: 275 | SEQ ID NO: 276 |
| | | QSSKSVYYNTHLP | RASTLAS | AGGYANDVDNG |
| 196 | H2 | SEQ ID NO: 281 | SEQ ID NO: 282 | SEQ ID NO: 283 |
| | | AYAIH | IIDAGGNIVYATWALG | GLSSYSNSAMDL |
| | L2 | SEQ ID NO: 284 | SEQ ID NO: 285 | SEQ ID NO: 286 |
| | | QASQNIYTYLA | QASTLAS | QSTAGNAYNA |
| 197 | H2 | SEQ ID NO: 291 | SEQ ID NO: 292 | SEQ ID NO: 293 |
| | | SYAMN | VITSSGTIYYANWATG | GITGAVTTGLSL |
| | L2 | SEQ ID NO: 294 | SEQ ID NO: 295 | SEQ ID NO: 296 |
| | | QASQNIYNNLA | AASNLAS | LGGYYGNNVGNT |
| 198 | H1 | SEQ ID NO: 301 | SEQ ID NO: 302 | SEQ ID NO: 303 |
| | | SYTMA | TIDSSGSADYASWAQS | PTNYEYTWALNI |
| | L2 | SEQ ID NO: 304 | SEQ ID NO: 305 | SEQ ID NO: 306 |
| | | QASQTIYSYLA | GASKLAS | QCTGYDSAYVYND |

CDRs are known to be responsible for antigen binding, however, it has been found that not all of the 6 CDRs are indispensable or unchangeable. In other words, it is possible to replace or change or modify one or more CDRs in the anti-CD40 antibody clones in Table 1, yet substantially retain the specific binding affinity to CD40.

Heavy chain CDR3 regions are located at the center of the antigen-binding site, and therefore are believed to make the most contact with antigen and provide the most free energy to the affinity of antibody to antigen. It is also believed that the heavy chain CDR3 is by far the most diverse CDR of the antigen-binding site in terms of length, amino acid composition and conformation by multiple diversification mechanisms (Tonegawa S, Nature (1983) 302:575-81). The diversity in the heavy chain CDR3 is sufficient to produce most antibody specificities (Xu J L, Davis M M, Immunity (2000) 13:37-45) as well as desirable antigen-binding affinity (Schier R, et al., J Mol Biol (1996) 263:551-67).

In certain embodiments, the anti-CD40 antibodies provided herein comprise suitable framework region (FR) sequences, as long as the antibodies and antigen-binding fragments thereof can specifically bind to CD40. The CDR sequences provided in Table 1 are obtained from rabbit antibodies, but they can be grafted to any suitable FR sequences of any suitable species such as mouse, human, rat, rabbit, among others, using suitable methods known in the art such as recombinant techniques.

In certain embodiments, the anti-CD40 antibodies provided herein are humanized. A humanized antibody or antigen-binding fragment is desirable in its reduced immunogenicity in human. A humanized antibody is chimeric in its variable regions, as non-human CDR sequences are grafted to human or substantially human FR sequences. Humanization of an antibody or antigen-binding fragment can be essentially performed by substituting the non-human (such as murine) CDR genes for the corresponding human CDR genes in a human immunoglobulin gene (see, for example, Jones et al., Nature (1986) 321:522-525; Riechmann et al., Nature (1988) 332:323-327; Verhoeyen et al., Science (1988) 239:1534-1536).

Suitable human heavy chain and light chain variable domains can be selected to achieve this purpose using methods known in the art. In an illustrative example, “best-fit” approach can be used, where a non-human (e.g., rodent) antibody variable domain sequence is screened or BLASTed against a database of known human variable domain sequences, and the human sequence closest to the non-human query sequence is identified and used as the human scaffold for grafting the non-human CDR sequences (see, for example, Sims et al., J Immunol (1993) 151:2296; Chothia et al., J Mot Biol (1987) 196:901). Alternatively, a framework derived from the consensus sequence of all human antibodies may be used for the grafting of the non-human CDRs (see, for example, Carter et al., Proc. Natl. Acad. Sci. USA (1992) 89:4285; Presta et al., J Immunol (1993) 151:2623).

In certain embodiments, the humanized antibodies or antigen-binding fragments provided herein are composed of substantially all human sequences except for the CDR sequences which are non-human. In some embodiments, the variable region FRs, and constant regions if present, are entirely or substantially from human immunoglobulin sequences. The human FR sequences and human constant region sequences may be derived different human immunoglobulin genes, for example, FR sequences derived from one human antibody and constant region from another human antibody. In some embodiments, the humanized antibody or antigen-binding fragment comprise human FR1-4.

In certain embodiments, the humanized antibodies and antigen-binding fragment thereof provided herein comprise one or more FR sequences of antibody clone HZD_3B5

The exemplary humanized anti-CD40 antibody clone HZD_3B5 retained the specific binding affinity to CD40-expressing cell, and are at least comparable to, or even better than, the parent rabbit antibodies in that aspect. The exemplary humanized antibody HZD_3B5 retained their functional interaction with CD40-expressing cell, in that both can induce human B cell activation and induce human dendritic cell maturation and activation.

In some embodiments, the FR regions derived from human may comprise the same amino acid sequence as the human immunoglobulin from which it is derived. In some embodiments, one or more amino acid residues of the human FR are substituted with the corresponding residues from the parent non-human antibody. This may be desirable in certain embodiments to make the humanized antibody or its fragment closely approximate the non-human parent antibody structure. In certain embodiments, the humanized antibody or antigen-binding fragment provided herein comprises no more than 10, 9, 8, 7, 6, 5, 4, 3, 2, or 1 amino acid residue substitutions in each of the human FR sequences, or no more than 10, 9, 8, 7, 6, 5, 4, 3, 2, or 1 amino acid residue substitutions in all the FRs of a heavy or a light chain variable domain. In some embodiments, such change in amino acid residue could be present in heavy chain FR regions only, in light chain FR regions only, or in both chains.

In certain embodiments, the anti-CD40 antibody provided herein comprises the VH and VL having amino acid sequences at least 90% or 95% identical to clone-paired sequences of Table 2. In certain embodiments, the anti-CD40 antibody provided herein comprises the VH and VL having amino acid sequences identical to clone-paired sequences of Table 2.

TABLE 2
Clone-Paired VH and VL Sequences

| Clone | VH | VL |
|---|---|---|
| 1D3 | H1 SEQ ID NO: 7 | L1 SEQ ID NO: 8 |
| | QSLEESGGRLVTPGTPLTLTCTASG | AEVVMTQTPSSVSAAVGGTVTFKCQ |
| | FSLSNYHMCWVRQAPGKGLEWIG | ASQSISSYLSWYQQKPGQPPKLLIYRA |
| | YNSYGGSAYYASWAKGRFTISKTS | STLESGVPSRFKGSGSGTQFTLTISDLE |
| | TTVDLKITRPTTEDTATYFCARAYL | CADAATYYCQTNYYDSGNTYGTFGG |
| | DYNGDMGFALWGPGTLVTVSS | GTEVVVK |
| 2B8 | H1 SEQ ID NO: 17 | L1 SEQ ID NO: 18 |
| | QSLEESGGRLVTPGTPLTLTCTVSGI | AVLTQTPSSVSAAVGGTVNIGCQSSK |
| | DLSSYAMGWVRQAPGKGLEYIGVI | SVYYNNWLAWYQQKPGQPPKLLIYR |
| | ASNSGISYYATWAEGRFTISKASTT | ASTLASGVPSRFKGSGSGTQFTLTISD |
| | VDLKITSPTTEDTATYFCVRGYIGD | VQCADATTYYCAGGYSSDSDTTFGG |
| | GALWGPGTLVTVSS | GTEVVVK |
| 3B12 | H1 SEQ ID NO: 27 | L2 SEQ ID NO: 28 |
| | QSLEESGGRLVTPGTPLTLTCTVSGI | AVLTQTPSSVSAAVGGTVTINCQSSK |
| | DLSSYAMGWVRQAPGKGLEYIGVI | TVYYNNWLAWYQQKPGQPPKLLIYR |
| | ASNSGISYYATWAEGRFTISKASTT | ASTLASGVPSRFKGSGSGTQFTLTISD |
| | VDLKITSPTTEDTATYFCVRGYIGD | VQCDDATTYYCAGGFSSDSDTTFGG |
| | GALWGPGTLVTVSS | GTEVVVK |
| 3B5 | H1 SEQ ID NO: 37 | L1 SEQ ID NO: 38 |
| | QSVEESGGRLITPGTPLTLTCTVSGF | ADIVMTQTPASVEAAVGGTVTIKCQA |
| | SLSTYAVSWVRQAPGKGLEWIGIID | SQSISNYLSWYQQKPGQPPKLLIYRAS |
| | SGGGTAYASWVKGRFTISKTSTTV | DLASGVPSRFKGSGSGTEFTLTISDLE |
| | DLKITSPTTEDTATYFCARGVTFFG | CADAATYYCQAYYYSGGGSYDYAFG |
| | LWGPGTLVTVSS | GGTEVVVK |
| 2E3 | H2 SEQ ID NO: 47 | L1 SEQ ID NO: 48 |
| | QSVEESGGRLVTPGTPLTLTCTVSG | ADVVMTQTPASVSAAVGGTVTIKCQ |
| | FSLSTYAMSWVRQAPGKGLEWIGII | ASQSISGVLAWYQQKPGQPPKLLIYR |
| | DGSSFTVYANWAKGRFTISKTSTTV | AADLASGVPSRFKGSGSGTEFTLTIND |
| | DLKMTSLTASDTATYFCARGATFF | LECDDAATYYCQSYYYSNSGSYSYAF |
| | GLWGPGTLVTVSS | GGGTEVVVK |
| 4A7 | H1 SEQ ID NO: 57 | L1 SEQ ID NO: 58 |
| | QSVEESGGRLVTPGTPLTLTCTVSG | ADIVMTQTPASVEAAVGDTVTIKCQA |
| | FSLSTYAMSWVRQAPGKGLEWIGII | SQSISSALAWYQQKPGQPPKLLIYRAS |
| | DGGGFAVYANWAKGRFTISKASTT | TLESGVPSRFKGSGSGTEFTLTISDLEC |
| | VDLKLPSPTTEDTATYFCARGVTFF | ADAATYYCQSYYYSSGGSYDYAFGG |
| | NLWGPGTLVTVSS | GTEVVVK |
| 3E12 | H2 SEQ ID NO: 67 | L1 SEQ ID NO: 58 |
| | QSVKESEGGLFKPTDTLTLTCTVSG | ADIVMTQTPASVSAAVGGTVTIKCQA |
| | FSFSNYRMIWVRQAPGNGLEWIGVI | SQNIGDNLAWHQQKPGQPPKLLIYGA |
| | DRNDNTYYATWAKSRSTITRNTNL | STLASGVPSRFKGSGSRTEYTLTISDL |
| | NTVTLKMTSLTAADTATYFCARDT | ECADAATYYCQSNYYSTSSSYYNSFG |
| | DYTTGGIWGPGTLVTVSS | GGTEVVVK |
| 4B7 | H2 SEQ ID NO: 77 | L2 SEQ ID NO: 78 |
| | QSVKESEGGLFKPTDTLTLTCTVSG | DIVMTQTPSSVSAAVGGTVTIKCQAS |
| | FTLSNYRVIWVRQAPGNGLEWIGGI | QNIGGNLAWYQQKPGQPPKLLIYGSS |
| | DRSAVAYYASWAKSRSTITRNTNL | TLESGVPSRFSGSGSGTEYTLTISDLQ |
| | NTVTLKMTSLTAADTATYFCARDT | CADAATYYCQSNYWSRSSSYVNSFG |
| | DYTSGGIWGPGTLVTVSS | GGTEVVVK |
| 4F11 | H2 SEQ ID NO: 87 | L2 SEQ ID NO: 88 |
| | QSLEESGGRLVTPGTPLTLTRTVSG | AYDMTQTPSPVSAAVGGTVTINCQAS |
| | FSLSSNALSWVRQAPGKGLEWIGII | EDIASNLAWYQQKPGQRPKLLIYGAS |
| | NSYGSTYYASWAKGRFTISKTSTTV | NLESGVPSRFKGSGSGTEYTLTISDLE |
| | DLRITSPTPEDTATYFCARVLEYSST | CADAATYYCQTSYYDSNTRNVFGGG |
| | DYGDWGYFNLWGPGTLVTVSS | TEVVVK |
| 6C2 | H1 SEQ ID NO: 97 | L2 SEQ ID NO: 98 |
| | QSVEESGGRLVTPGTPLTLTCTASG | AVLTQTPSPVSAAVGGTVSISCQSSQS |
| | FSLSTYYMIWVRQAPGKGLDYIGII | VRDNNWLSWFQQKPGHPPKLLIYGA |
| | SSNGNAYYATWAKGRFTISKTSTT | SNLESGVPSRFSGSGSGTEFTLTISDVQ |
| | VDLKMTSPTTEDTATYFCARAASIA | CDDAATYYCQGGFSTYSDNSFGGGT |
| | NSYYFNVWGPGTLVTVSS | EVVVK |
| 6E1 | H2 SEQ ID NO: 107 | L2 SEQ ID NO: 108 |
| | QSVEESGGRLVTPGTPLTLTCTVSG | AVLTQTPSSVEAAVGGTVSISCQSSKS |
| | FSLSSYAMTWVRQAPGKGLEWLGI | VYYKNWLAWYQQKPGQPPKLLIYGA |
| | IDTIGSTYYASWAKGRFTISKTSTTV | STLASGVPSRFKGSGSGAQFTLTISDV |
| | DLKITSPTTEDTATYFCVRDYIGSTS | QCDDAATYYCAGGYSSDSDTGFGGG |
| | GKKLWGRGTLVTVSS | TEVVVK |
| 6G8 | H2 SEQ ID NO: 117 | L1 SEQ ID NO: 118 |
| | QSVEESGGRLVTPGTPLTLTCTASG | ADIVMTQTPASVSEPVGGTVTIKCQA |
| | FSLSTYYMTWVRQAPGKGLEAIGY | SESISSNLAWYQQKPGQPPKFLIYRAS |
| | INTGGSTYYASRAKGRFTISKTSTT | TLASGVPSRFKGSGFGTQFTLTISDLE |
| | VDLKMTSPTTEDTATYFCARADAS | CADAATYYCQSNYGSISSSYGNAFGG |
| | EHNYNGLNIWGPGTLVTVSS | GTEVVVK |
| 6H9 | H2 SEQ ID NO: 127 | L2 SEQ ID NO: 128 |
| | QSLEESGGRLVTPGTPLTLTCTVSGI | AVLTQTPSSVSAAVGGTVTINCQSSK |
| | DLSSYAMGWVRQAPGKGLEYIGVI | TVYYNNWLAWYQQKPGQPPKLLIYR |
| | ASNSGISYYATWAEGRFTISKASTT | ASTLASGVPSRFKGSGSGTQFTLTISD |
| | VDLKITSPTTEDTATYFCVRGYIGD | VQCDDATTYYCAGGFSSDSDTTFGG |
| | GALWGPGTLVTVSS | GTEVVVK |
| 7A6 | H2 SEQ ID NO: 137 | L2 SEQ ID NO: 138 |
| | QSVEESGGRLVTPGTPLTLACTVSG | ADIVMTQTPASVEAAVGGTVTIKCQA |
| | IDLSTWTMSWVRQAPGKGLEWIGII | SQSIYRSLAWYQQKPGQPPKLLMYH |
| | NSDSNTYYASWAKGRFTISKASTT | ASDLASGVPSRFKGSGSGTEYTLTISD |
| | VDLKMTSLTTEDTATYFCARGVGG | LECADAATYYCQSYYYSGINTYGNAF |
| | YSSGPDYLDIWGPGTLVTVSS | GGGTEVVVK |
| 7C11 | H2 SEQ ID NO: 147 | L2 SEQ ID NO: 148 |
| | QSLEESGGRLVTPGGSLTLTCTVSGI | AFELTQTPASVEAGVGGTVTIKCQAS |
| | DLSTNGMGWVRQGPGKGLEWIGTI | QSIDRYLAWYQQKSGQPPKLLISYTS |
| | RVTAGTDYANWAKGRFTISKTSST | KLASGVPSRFKGSGSGTEFTLTISDLE |
| | TVDLKMTSLTTEDTATYFCARGHA | CADAATYYCQSYFTSDDANSFGGGT |
| | NNDAYVYFNLWGQGTLVTVSS | EVVVK |
| 8C10 | H1 SEQ ID NO: 157 | L1 SEQ ID NO: 158 |
| | QSLAESGGRLVTPGGSLTLTCTVSG | AFELTQTPASVEAGVGGTVTIKCQAS |
| | IDLSTDGMGWVRQGPGKGLEWIGT | QSIDRYLAWYQQKPGQPPKLLMAAA |
| | IRVTGGTDFATWAKGRFTIAKTSST | ANLASGVPSRFKGSRSGTEFTLTISDL |
| | TVDLKMTSLTTEDTATYFCARGHA | ECADGATYYCQSYFTSDDGNTFGGG |
| | NNDAYVYFNLWGQGTLVTVSS | TEVVVK |
| 2C10 | H2 SEQ ID NO: 167 | L4 SEQ ID NO: 168 |
| | QSLEESGGRLVTPGTPLTLTCTASG | DVVMTQTPASVSGAVGGTVTIKCQA |
| | FTMNRYRMSWVRQAPGKGLEWIG | SENIFSYLAWYQQKPGQPPKLLFYGA |
| | TINNDAGASYASWVNGRFTISKTSS | SNLASGVPSRFSGSRSGTEFTLTISDLE |
| | TTVDLKITGPTTEDTATYFCARAGD | CADAATYYCQFTNYGSSDIRSPFGGG |
| | NIYVDLYNLWGPGTLVTVSS | TEVVVK |
| 7G6 | H2 SEQ ID NO: 177 | L2 SEQ ID NO: 178 |
| | QSLEESGGRLVTPGGSLTLTCTVSGI | AFELTQTPASVEAGVGGTVTIKCQAS |
| | DLSTNGMGWVRQGPGKGLEWIGTI | QSIDRYLAWYQQKPGQPPKLLISYTST |
| | RVTGGTDYATWAKGRFTISKTSSTT | LTSGVPSRFKGSGSGTEFTLTISDLEC |
| | VDLKMTSPTTEDTATYFCARGHAN | ADAATYYCQSYFTSDDANTFGGGTE |
| | NDAYVYFNLWGQGTLVTVSS | VVVK |
| 7H11 | H2 SEQ ID NO: 187 | L2 SEQ ID NO: 188 |
| | QSVEESGGRLVTPGTPLTLTCTVSGI | DPVLTQTPSSASEPVGGTVTIKCQASQ |
| | DFSKYWMAWVRQAPGEGLEYIGIL | SIGSYLAWYQQKPGQPPKLLIYSPSTL |
| | STTGSTYYASWAKGRFTISKTSTTV | ASGVSSRFKGSRSGTEFTLTISDLECA |
| | DLKITSLTTEDTATYFCARGNYIGN | DAATYYCQSTAVGITFGGGTEVVVK |
| | GYTWDIWGPGTLVTVSS | |
| 8C6 | H1 SEQ ID NO: 197 | L2 SEQ ID NO: 198 |
| | QSVEESGGRLVTPGTPLTLTCTASG | DVAMTQTPSSVSAAVGGTVTIECQAS |
| | FTITSYYIIWVRQAPGEGLEYIGIIDT | QNIYTNLAWYQQKPGQRPKLLIYYAS |
| | GGSADYASWAKGRFTISRTSTTVD | TLASGVPSRFSGSGSGTEFSLTISDLEC |
| | LKMTSLTTEDTATYFCARADVGSV | ADAATYYCQCTYIKWRHVCGFGGGT |
| | GSIWGPGTLVTVSS | EVVVK |
| 7D2 | H2 SEQ ID NO: 207 | L1 SEQ ID NO: 208 |
| | QSVEESGGRLVTPGTPLTLTCTVSG | AVLTQTPSPVSAAVEGTVTINCQSSQS |
| | FSLNNYYMGWVRQAPGKGLEYIG | VDNNRLSWFQQKPGQPPKLLIYDAST |
| | WITSGRIAAYANWAKGRFTISKTST | LASGVPPRFKGSGSGTQFTLTISDVQC |
| | TVDLKITSPTIGDTATHFCATTTTSD | DDAATYYCAGGYSFPSENAFGGGTE |
| | AYFFPRLGLWGQGTLVTVSS | VVVK |
| 7F10 | H1 SEQ ID NO: 217 | L1 SEQ ID NO: 218 |
| | QSVEESGGRLVTPGTPLTLTCTASG | AYDMTQTPASVEAAVGGTVTIKCQA |
| | FSLSDYYMTWVRQAPGKGLEWIGII | SQSISSYCSWYQQKPGQPPKLLIYRAS |
| | SSSGNTYNATWANGRFTISKTSTTV | ILQSGVPSRFKGSGSGTDFTLTISDLEC |
| | DLKITSPTTEDTATYFCAREDGSGG | ADAATYYCQQGSTIANIENTFGGGXX |
| | RFDAFDLWGPGTLVTVSS | VVVK |
| 2E11 | H1 SEQ ID NO: 227 | L2 SEQ ID NO: 228 |
| | QSLEESGGRLVTPGTPLTLTCTVSGI | AVLTQTPSPVSAAVGGTVSISCQSSKT |
| | DLSRYAMGWVRQAPGKGLEFIGIIT | VYYNNYLSWFQQKPGQPPKELIYYAS |
| | SNSGDTYYATWAKGRFTISKTSTTV | TLSSGVPSRFKGSGSGTQFTLTISDVQ |
| | DLKITSPTTEDTATYFCVRGYIGAP | CDDAATYYCAGGYNSDSDNTFGGGT |
| | NLWGPGTLVTVSS | EVVVK |
| 5F9 | H2 SEQ ID NO: 237 | L2 SEQ ID NO: 238 |
| | QSVEESGGRLVTPGTPLTLTCTASG | ADIVMTQTPASVEAAVGGTVTIKCQA |
| | FSLIGYVMGWFRQAPGKGLEYIGW | SQSISSRLAWYQLKPGQPPKLLIYSAS |
| | ISFGGDPYYASWVKGRFTISKTSTT | TLASGVPSRFKGSGSGTEFTLTISDLE |
| | VDLKMTSLTTEDTATYFCARSTYG | CADAATYYCQNHGYATSESYTNTFG |
| | DIWGPGTLVTVSS | GGTEVVVK |
| 5D10 | H10 SEQ ID NO: 247 | L9 SEQ ID NO: 248 |
| | QSVEESGGRLVTPGTPLTLTCTVSG | ADIVMTQTPASVEAGVGGTVTIKCQA |
| | FSFYDHTMIWVRQAPGKGLEYIGFI | SQTIYSYLSWYQQKPGQPPKLLIYRAS |
| | NIGGRAYYASWAKGRFTISRTSTTV | TLASGVPSRFRGSGSGTEFTLTISDLEC |
| | DLEMTSLTTEDTATYFCARGDTHG | ADAAAYYCQNYYDSTSSNAFGKGTE |
| | YSYPEIWGPGTLVTVSS | VVVK |
| 192 | H2 SEQ ID NO: 257 | L2 SEQ ID NO: 258 |
| | QSVEESGGRLVTPGTPLTLTCTVSG | AVLTQTPSPVSAGVGGTVTISCQSSKS |
| | FSLSSYAMNWVRQAPGKGLEYTG | VYYNTHLSWFQQKPGQPPKLLIYRAS |
| | MIATGGKTFYAGWAEGRFTISKTST | TLASGVPSRFKGSGSGTQFTLTISDVQ |
| | TVDLHIASLTTEDTATYYCARGYV | CDDAATYYCAGGYANDVDNGFGGG |
| | GDIYFGFWGPGTLVTVSS | TEVVVK |
| 194 | H2 SEQ ID NO: 267 | L1 SEQ ID NO: 268 |
| | QSVEESGGRLVTPGTPLTLTCTVSG | AVLTQTPSPVSAAVGGTVSISCQSSKS |
| | FSLSSYAMSWVRQAPGKGLEYIGV | VYYNNYLSWFQQKPGQPPKQLIYLAS |
| | MNTGGSAHYATWAKGRFTISKTST | TLASGVPSRFKGSGSGTEFTLTISDVQ |
| | TVDLKITSPTTADTATYFCARGYIG | CADAATYYCAGGYTDSDDAFGGGTE |
| | SPNLWGPGTLVTVSS | VVVK |
| 195 | H2 SEQ ID NO: 277 | L1 SEQ ID NO: 278 |
| | QSVEESGGRLVTPGTPLTLTCTVSG | AVLTQTPSPVSAGVGGTVTISCQSSKS |
| | FSLSSYAMNWVRQAPGKGLEYIGM | VYYNTHLPWFQQKPGQPPKLLIYRAS |
| | IATGGKTFYAGWAEGRFTISKTSTT | TLASGVPSRFKGSGSGTQFTLTISDVQ |
| | VDLHIASLTTEDTATYYCARGYVG | CDDAATYYCAGGYANDVDNGFGGG |
| | DIYFGFWGPGTLVTVSS | TEVVVK |
| 196 | H2 SEQ ID NO: 287 | L2 SEQ ID NO: 288 |
| | QSVEESGGRLVTPGTPLTLTCTVSG | DVVMTQTPASVSEPVGGTVTMKCQA |
| | FSLSAYAIHWVRQAPGKGLEYIGII | SQNIYTYLAWYQQKPGRPPKLLIYQA |
| | DAGGNIVYATWALGRFTISKTSTTV | STLASGVSSRFSGSGSGTEFTLTISDLE |
| | DLKITSPTTEDTATYFCGRGLSSYS | CADAATYYCQSTAGNAYNAFGGGTE |
| | NSAMDLWGPGTLVTVSS | VVVK |
| 197 | H2 SEQ ID NO: 297 | L2 SEQ ID NO: 298 |
| | QEQLKESGGGLVTPGTALTLTCTAS | DVVMTQTPPSVSEPVGGTVTIKCQAS |
| | GSDISSYAMNWVRQAPGKGLEYIG | QNIYNNLAWYQQTPGQPPKLLGYAA |
| | VITSSGTIYYANWATGRFTISKASST | SNLASGVSSRFSGSGYGTEFTLTISDL |
| | TVDLKTTRLTTEDTATYFCARGITG | ECADAATYYCLGGYYGNNVGNTFGG |
| | AVTTGLSLWGPGTLVTVSS | GTEVVVK |
| 198 | H1 SEQ ID NO: 307 | L2 SEQ ID NO: 308 |
| | QSVKESEGGLFKPTDTLTLTCTVSG | DVVMTQTPASVSEPVGGTVTIKCQAS |
| | FSLGSYTMAWVRQAPGNGLEYIGT | QTIYSYLAWYQQKPGQPPKLLIYGAS |
| | IDSSGSADYASWAQSRSTITRNTNL | KLASGVPSRFSGSRSGTEFTLTISDLEC |
| | NTVTLKMTSLTAADTATYFCARPT | ADAATYYCQCTGYDSAYVYNDFGG |
| | NYEYTWALNIWGPGTLVTVSS | GTEVVVK |
| HZD_3B5 | VH SEQ ID NO: 311 | VK SEQ ID NO: 312 |
| | EVQLVESGGGLIQPGGSLRLSCAVS | DIVMTQSPSTLSASVGDRVTITCQASQ |
| | GFSLSTYAVSWVRQAPGKGLEWV | SISNYLSWYQQKPGKAPKLLIYRASD |
| | GIIDSGGGTAYASWVKGRFTISRDN | LASGVPSRFSGSGSGTEFTLTISSLQPD |
| | SKNTLYLQMNSLRAEDTAVYYCAR | DFATYYCQAYYYSGGGSYDYAFGGG |
| | GVTFFGLWGQGTLVTVSS | TKVEIK |

In certain embodiments, the anti-CD40 antibodies provided herein further comprise an immunoglobulin constant region. In some embodiments, an immunoglobulin constant region comprises a heavy chain and/or a light chain constant region. The heavy chain constant region comprises CH1, hinge, and/or CH2-CH3 regions. In certain embodiments, the heavy chain constant region comprises an Fc region. In certain embodiments, the light chain constant region comprises Cκ or Cλ.

The antibodies or antigen-binding fragments thereof provided herein can be a monoclonal antibody, polyclonal antibody, humanized antibody, chimeric antibody, recombinant antibody, bispecific antibody, labeled antibody, bivalent antibody, or anti-idiotypic antibody. A recombinant antibody is an antibody prepared in vitro using recombinant methods rather than in animals.

### III. Antibody Variants

The antibodies and antigen-binding fragments thereof provided herein also encompass various variants thereof. In certain embodiments, the antibodies and antigen-binding fragments thereof encompasses various types of variants of an exemplary antibody provided herein, i.e., antibody clone listed in Tables 1 and 2.

In certain embodiments, the antibody variants comprise one or more modifications or substitutions in one or more CDR sequences as provided in Table 1, one or more variable region sequences (but not in any of the CDR sequences) provided in Table 2, and/or the constant region (e.g., Fc region). Such variants retain specific binding affinity to CD40 of their parent antibodies, but have one or more desirable properties conferred by the modification(s) or substitution(s). For example, the antibody variants may have improved antigen-binding affinity, improved glycosylation pattern, reduced risk of glycosylation, reduced deamination, reduced or depleted effector function(s), improved FcRn receptor binding, increased pharmacokinetic half-life, pH sensitivity, and/or compatibility to conjugation (e.g., one or more introduced cysteine residues).

The parent antibody sequence may be screened to identify suitable or preferred residues to be modified or substituted, using methods known in the art, for example “alanine scanning mutagenesis” (see, for example, Cunningham and Wells, Science (1989) 244:1081-1085). Briefly, target residues (e.g., charged residues such as Arg, Asp, His, Lys, and Glu) can be identified and replaced by a neutral or negatively charged amino acid (e.g., alanine or polyalanine), and the modified antibodies are produced and screened for the interested property. If substitution at a particular amino acid location demonstrates an interested functional change, then the position can be identified as a potential residue for modification or substitution. The potential residues may be further assessed by substituting with a different type of residue (e.g., cysteine residue, positively charged residue, etc.).

Affinity Variant

Affinity variant may contain modifications or substitutions in one or more CDR sequences as provided in Table 1, one or more FR sequences, or the heavy or light chain variable region sequences provided in Table 2. FR sequences can be readily identified by a skilled person in the art based on the CDR sequences in Table 1 and variable region sequences in Table 2, as it is well-known in the art that a CDR region is flanked by two FR regions in the variable region. The affinity variants retain specific binding affinity to CD40 of the parent antibody, or even have improved CD40 specific binding affinity over the parent antibody. In certain embodiments, at least one (or all) of the substitution(s) in the CDR sequences, FR sequences, or variable region sequences comprises a conservative substitution.

A skilled artisan will understand that in the CDR sequences provided in Table 1 and variable region sequences provided in Table 2, one or more amino acid residues may be substituted yet the resulting antibody or antigen-binding fragment still retain the binding affinity to CD40, or even have an improved binding affinity. Various methods known in the art can be used to achieve this purpose. For example, a library of antibody variants (such as Fab or scFv variants) can be generated and expressed with phage display technology, and then screened for the binding affinity to human CD40. For another example, computer software can be used to virtually simulate the binding of the antibodies to human CD40, and identify the amino acid residues on the antibodies which form the binding interface. Such residues may be either avoided in the substitution so as to prevent reduction in binding affinity, or targeted for substitution to provide for a stronger binding.

In certain embodiments, the humanized antibody or antigen-binding fragment provided herein comprises one or more amino acid residue substitutions in one or more CDR sequences, and/or one or more FR sequences. In certain embodiments, an affinity variant comprises no more than 10, 9, 8, 7, 6, 5, 4, 3, 2, or 1 substitutions in the CDR sequences and/or FR sequences in total.

In certain embodiments, the anti-CD40 antibodies and antigen-binding fragments thereof comprise 1, 2, or 3 CDR sequences having at least 80% (e.g., at least 85%, 88%, 90%, 91%, 92%, 93%, 94%, 95%, 96%, 97%, 98%, 99%) sequence identity to that (or those) listed in Table 1, and in the meantime retain the binding affinity to CD40 at a level similar to or even higher than its parent antibody.

In certain embodiments, the anti-CD40 antibody provided herein comprises: (a) a heavy chain variable region comprising heavy chain complementarity determining region (HCDR)1 of amino acid sequence TYAX₁S (SEQ ID NO: 313), wherein X₁ is M or V, HCDR2 of amino acid sequence IIDX₂X₃X₄X₅X₆X₇YAX₈WX₉KG (SEQ ID NO: 314), wherein each of X₂, X₃ and X₄ is independently G or S, X₅ is F or G, X₆ is A or T, X₇ is A or V, X₈ is N or S, X₉ is A or V, and HCDR3 of amino acid sequence GX₁₀TFFX₁₁L (SEQ ID NO: 315), wherein X₁₀ is A or V, and X₁₁ is G or N; and (b) a light chain variable region comprising light chain complementarity determining region (LCDR)1 of amino acid sequence QASQSISX₁₂X₁₃LX₁₄ (SEQ ID NO: 316), wherein X₁₂ is G, N or S, X₁₃ is A, V or Y, and X₁₄ is A or S, LCDR2 of amino acid sequence RAX₁₅X₁₆LX₁₇S (SEQ ID NO: 317), wherein each of X₁₅ is A or S, X₁₆ is D or T, X₁₇ is A or E, and LCDR3 of amino acid sequence QX₁₈YYYSX₁₉X₂₀GSYX₂₁YA (SEQ ID NO: 318), wherein X₁₈ is A or S, X₁₉ is G, N or S, X₂₀ is G or S, and X₂₁ is D or S.

In certain embodiments, the anti-CD40 antibodies and antigen-binding fragments thereof comprise one or more variable region sequences having at least 80% (e.g., at least 85%, 88%, 90%, 91%, 92%, 93%, 94%, 95%, 96%, 97%, 98%, 99%) sequence identity to that (or those) provided herein, and in the meantime retain the binding affinity to CD40 at a level similar to or even higher than its parent antibody. In some embodiments, a total of 1 to 10 amino acids have been substituted, inserted, or deleted in a variable region sequence listed in Table 2. In some embodiments, the substitutions, insertions, or deletions occur in regions outside the CDRs (e.g., in the FRs).

Glycosylation Variant

The anti-CD40 antibodies and antigen-binding fragments provided herein also encompass a glycosylation variant, which can be obtained to either increase or decrease the extent of glycosylation of the antibody or antigen binding fragment.

The antibody or antigen binding fragment thereof may comprise one or more amino acid residues with a side chain to which a carbohydrate moiety (e.g., an oligosaccharide structure) can be attached. Glycosylation of antibodies is typically either N-linked or O-linked. N-linked refers to the attachment of the carbohydrate moiety to the side chain of an asparagine residue, for example, an asparagine residue in a tripeptide sequence such as asparagine-X-serine and asparagine-X-threonine, where X is any amino acid except proline. O-linked glycosylation refers to the attachment of one of the sugars N-aceylgalactosamine, galactose, or xylose to a hydroxyamino acid, most commonly to serine or threonine. Removal of a native glycosylation site can be conveniently accomplished, for example, by altering the amino acid sequence such that one of the above-described tripeptide sequences (for N-linked glycosylation sites) or serine or threonine residues (for O-linked glycosylation sites) present in the sequence in the is substituted. A new glycosylation site can be created in a similar way by introducing such a tripeptide sequence or serine or threonine residue.

Cysteine-Engineered Variant

The anti-CD40 antibodies and antigen-binding fragments provided herein also encompass a cysteine-engineered variant, which comprises one or more introduced free cysteine amino acid residues.

A free cysteine residue is one which is not part of a disulfide bridge. A cysteine-engineered variant is useful for conjugation with for example, a cytotoxic and/or imaging compound, a label, or a radioisoptype among others, at the site of the engineered cysteine, through for example a maleimide or haloacetyl. Methods for engineering antibodies or antigen-binding fragments to introduce free cysteine residues are known in the art, see, for example, WO2006/034488.

Fc Variant

The anti-CD40 antibodies and antigen-binding fragments provided herein also encompass an Fc variant, which comprises one or more amino acid residue modifications or substitutions at its Fc region and/or hinge region.

In certain embodiments, the anti-CD40 antibodies or antigen-binding fragments comprise one or more amino acid substitution(s) that improves pH-dependent binding to neonatal Fc receptor (FcRn). Such a variant can have an extended pharmacokinetic half-life, as it binds to FcRn at acidic pH which allows it to escape from degradation in the lysosome and then be translocated and released out of the cell. Methods of engineering an antibody and antigen-binding fragment thereof to improve binding affinity with FcRn are well-known in the art, see, for example, Vaughn, D. et al, Structure, 6(1): 63-73, 1998; Kontermann, R. et al, Antibody Engineering, Volume 1, Chapter 27: Engineering of the Fc region for improved PK, published by Springer, 2010; Yeung, Y. et al, Cancer Research, 70: 3269-3277 (2010); and Hinton, P. et al, J. Immunology, 176:346-356 (2006).

In certain embodiments, the anti-CD40 antibodies or antigen-binding fragments comprise one or more amino acid substitution(s) that alters the antibody-dependent cellular cytotoxicity (ADCC). Certain amino acid residues at CH2 domain of the Fc region can be substituted to provide for enhanced ADCC activity. Alternatively or additionally, carbohydrate structures on the antibody can be changed to enhance ADCC activity. Methods of altering ADCC activity by antibody engineering have been described in the art, see for example, Shields R L. et al., J Biol Chem (2001) 276(9): 6591-604; Idusogie E E. et al., J Immunol (2000) 164(8):4178-84; Steurer W. et al., J Immunol (1995) 155(3): 1165-74; Idusogie E E. et al., J Immunol (2001) 166(4): 2571-5; Lazar G A. et al., PNAS (2006) 103(11): 4005-4010; Ryan M C. et al., Mol. Cancer Ther (2007) 6: 3009-3018; Richards J O, et al., Mol Cancer Ther (2008) 7(8): 2517-27; Shields R. L. et al, J Biol Chem (2002) 277: 26733-26740; Shinkawa T. et al, J Biol Chem (2003) 278: 3466-3473.

In certain embodiments, the anti-CD40 antibodies or antigen-binding fragments comprise one or more amino acid substitution(s) that alters Complement Dependent Cytotoxicity (CDC), for example, by improving or diminishing C1q binding and/or CDC (see, for example, WO99/51642; Duncan & Winter, Nature (1988) 322:738-40; U.S. Pat. Nos. 5,648,260; 5,624,821); and WO94/29351 concerning other examples of Fc region variants.

In certain embodiments, the anti-CD40 antibodies or antigen-binding fragments comprise one or more amino acid substitution(s) in the interface of the Fc region to facilitate and/or promote heterodimerization. These modifications comprise introduction of a protuberance into a first Fc polypeptide and a cavity into a second Fc polypeptide, wherein the protuberance can be positioned in the cavity so as to promote interaction of the first and second Fc polypeptides to form a heterodimer or a complex. Methods of generating antibodies with these modifications are known in the art, e.g., as described in U.S. Pat. No. 5,731,168.

### IV. Antigen-Binding Fragments

Provided herein are also anti-CD40 antigen-binding fragments. Various types of antigen-binding fragments are known in the art and can be developed based on the anti-CD40 antibodies provided herein, including for example, the exemplary antibodies whose CDR are shown in Table 1 and variable sequences are shown in Table 2, and their different variants (such as affinity variants, glycosylation variants, Fc variants, cysteine-engineered variants and so on).

In certain embodiments, an anti-CD40 antigen-binding fragment provided herein is a camelized single domain antibody, a diabody, a single chain Fv fragment (scFv), an scFv dimer, a BsFv, a dsFv, a (dsFv)₂, a dsFv-dsFv′, an Fv fragment, a Fab, a Fab′, a F(ab′)₂, a bispecific antibody, a ds diabody, a nanobody, a domain antibody, a single domain antibody, or a bivalent domain antibody.

Various techniques can be used for the production of such antigen-binding fragments. Illustrative methods include, enzymatic digestion of intact antibodies (see, e.g., Morimoto et al., Journal of Biochemical and Biophysical Methods (1992) 24:107-117; and Brennan et al., Science (1985) 229:81), recombinant expression by host cells such as E. Coli (e.g., for Fab, Fv and ScFv antibody fragments), screening from a phase display library as discussed above (e.g., for ScFv), and chemical coupling of two Fab′-SH fragments to form F(ab′)2 fragments (Carter et al., Bio/Technology (1992) 10:163-167). Other techniques for the production of antibody fragments will be apparent to a skilled practitioner.

Single Chain Variable Fragment

A Single Chain Variable Fragment (scFv) is a fusion of the variable regions of the heavy and light chains of immunoglobulins, linked together with a short (usually serine, glycine) linker. This chimeric molecule retains the specificity of the original immunoglobulin, despite removal of the constant regions and the introduction of a linker peptide. This modification usually leaves the specificity unaltered. These molecules were created historically to facilitate phage display where it is highly convenient to express the antigen binding domain as a single peptide. Alternatively, scFv can be created directly from subcloned heavy and light chains derived from a hybridoma. Single chain variable fragments lack the constant Fc region found in complete antibody molecules, and thus, the common binding sites (e.g., protein A/G) used to purify antibodies. These fragments can often be purified/immobilized using Protein L since Protein L interacts with the variable region of kappa light chains.

Flexible linkers generally are comprised of helix- and turn-promoting amino acid residues such as alanine, serine and glycine. However, other residues can function as well. Tang et al. (1996) used phage display as a means of rapidly selecting tailored linkers for single-chain antibodies (scFvs) from protein linker libraries. A random linker library was constructed in which the genes for the heavy and light chain variable domains were linked by a segment encoding an 18-amino acid polypeptide of variable composition. The scFv repertoire (approx. 5×10⁶ different members) was displayed on filamentous phage and subjected to affinity selection with hapten. The population of selected variants exhibited significant increases in binding activity but retained considerable sequence diversity. Screening 1054 individual variants subsequently yielded a catalytically active scFv that was produced efficiently in soluble form. Sequence analysis revealed a conserved proline in the linker two residues after the VH C terminus and an abundance of arginines and prolines at other positions as the only common features of the selected tethers.

In a separate embodiment, a single-chain antibody can be created by joining receptor light and heavy chains using a non-peptide linker or chemical unit. Generally, the light and heavy chains will be produced in distinct cells, purified, and subsequently linked together in an appropriate fashion (i.e., the N-terminus of the heavy chain being attached to the C-terminus of the light chain via an appropriate chemical bridge).

Cross-linking reagents are used to form molecular bridges that tie functional groups of two different molecules, e.g., a stabilizing and coagulating agent. However, it is contemplated that dimers or multimers of the same analog or heteromeric complexes comprised of different analogs can be created. To link two different compounds in a step-wise manner, hetero-bifunctional cross-linkers can be used that eliminate unwanted homopolymer formation.

An exemplary hetero-bifunctional cross-linker contains two reactive groups: one reacting with primary amine group (e.g., N-hydroxyl succinimide) and the other reacting with a thiol group (e.g., pyridyl disulfide, maleimides, halogens, etc.). Through the primary amine reactive group, the cross-linker may react with the lysine residue(s) of one protein (e.g., the selected antibody or fragment) and through the thiol reactive group, the cross-linker, already tied up to the first protein, reacts with the cysteine residue (free sulfhydryl group) of the other protein (e.g., the selective agent).

It is preferred that a cross-linker having reasonable stability in blood will be employed. Numerous types of disulfide-bond containing linkers are known that can be successfully employed to conjugate targeting and therapeutic/preventative agents. Linkers that contain a disulfide bond that is sterically hindered may prove to give greater stability in vivo, preventing release of the targeting peptide prior to reaching the site of action. These linkers are thus one group of linking agents.

Another cross-linking reagent is SMPT, which is a bifunctional cross-linker containing a disulfide bond that is “sterically hindered” by an adjacent benzene ring and methyl groups. It is believed that steric hindrance of the disulfide bond serves a function of protecting the bond from attack by thiolate anions such as glutathione which can be present in tissues and blood, and thereby help in preventing decoupling of the conjugate prior to the delivery of the attached agent to the target site.

The SMPT cross-linking reagent, as with many other known cross-linking reagents, lends the ability to cross-link functional groups such as the SH of cysteine or primary amines (e.g., the epsilon amino group of lysine). Another possible type of cross-linker includes the hetero-bifunctional photoreactive phenylazides containing a cleavable disulfide bond such as sulfosuccinimidyl-2-(p-azido salicylamido) ethyl-1,3′-dithiopropionate. The N-hydroxy-succinimidyl group reacts with primary amino groups and the phenylazide (upon photolysis) reacts non-selectively with any amino acid residue.

In addition to hindered cross-linkers, non-hindered linkers also can be employed in accordance herewith. Other useful cross-linkers, not considered to contain or generate a protected disulfide, include SATA, SPDP and 2-iminothiolane (Wawrzynczak & Thorpe, Methods for preparing immunotoxins: effect of the linkage on activity and stability. In: Vogel C-W (ed) Immunoconjugates: antibody conjugates in radioimaging and therapy of cancer. (1987) Oxford University Press, New York, p 28). The use of such cross-linkers is well understood in the art. Another embodiment involves the use of flexible linkers.

U.S. Pat. No. 4,680,338 describes bifunctional linkers useful for producing conjugates of ligands with amine-containing polymers and/or proteins, especially for forming antibody conjugates with chelators, drugs, enzymes, detectable labels and the like. U.S. Pat. Nos. 5,141,648 and 5,563,250 disclose cleavable conjugates containing a labile bond that is cleavable under a variety of mild conditions. This linker is particularly useful in that the agent of interest may be bonded directly to the linker, with cleavage resulting in release of the active agent. Particular uses include adding a free amino or free sulfhydryl group to a protein, such as an antibody, or a drug.

U.S. Pat. No. 5,856,456 provides peptide linkers for use in connecting polypeptide constituents to make fusion proteins, e.g., single chain antibodies. The linker is up to about 50 amino acids in length, contains at least one occurrence of a charged amino acid (preferably arginine or lysine) followed by a proline, and is characterized by greater stability and reduced aggregation. U.S. Pat. No. 5,880,270 discloses aminooxy-containing linkers useful in a variety of immunodiagnostic and separative techniques.

Bispecific Antibody

In certain embodiments, the anti-CD40 antibody disclosed herein is a bispecific antibody. In certain embodiments, the anti-CD40 bispecific antibody can be used to treat tumor by specifically activating CD40 signaling in the tumor microenvironment. In some embodiments, the anti-CD40 bispecific antibody is against an antigen enriched in tumor microenvironment. In certain embodiments, the anti-CD40 bispecific antibody provided herein shows low basal activity in activating CD40 signaling when the antibody does not bind to the antigen enriched in the tumor microenvironment. After the anti-CD40 bispecific antibody binds to the antigen in the tumor microenvironment, the bispecific antibody is enriched and clusters CD40 on the surface of a cell, thus activating the CD40 signaling. As a result, the anti-CD40 bispecific antibody provided herein can activate the immune response against tumor cells without inducing systemic immune response that leads to adverse effects.

In some embodiments, the antigen enriched in tumor microenvironment is a tumor antigen. Examples of tumor antigen include alphafetoprotein (AFP), carcinoembryonic antigen (CEA), CA-125, MUC-1, epithelial tumor antigen (ETA), tyrosinase, and melanoma-associated antigen (MAGE). In some embodiments, the anti-CD40 bispecific antibody is against an immune checkpoint. Immune checkpoints include, for example: A2AR, B7.1, B7.2, B7-H2, B7-H3, B7-H4, B7-H6, BTLA, CD48, CD160, CD244, CTLA-4, ICOS, LAG-3, LILRB1, LILRB2, LILRB4, OX40, PD-1, PD-L1, PD-L2, SIRPalpha (CD47), TIGIT, TIM-3, TIM-1, TIM-4, and VISTA.

The antigen-binding portion of the anti-CD40 bispecific antibody against the antigen enriched in tumor microenvironment or immune checkpoint can be generated based on the antibodies known in the art. For example, an antigen-binding portion against PD-L1 can be generated based on the antibodies disclosed in PCT application publication No. WO2016/022630.

It is appreciated that the anti-CD40 bispecific antibodies of the present disclosure can have various forms and structures. In certain embodiments, anti-CD40 bispecific antibodies could be constructed in many ways as reviewed by Konterman et al., MAbs (2017) 9:182-212. In particular, anti-CD40 bispecific antibodies could be constructed as covalent antibody conjugates, asymmetric F(ab′)₂, CovX-bodies, mouse/rat chimeric IgGs, Kλ-bodies with common heavy chains, tandem single-chain variable domains (scFv), BiTEs, triplebodies, diabodies, tandem domain antibodies, scFv fusions with CH1/CL domains, Fab-scFv bibodies or tribodies, Fab-Fv fusion, Fab-single domain antibody (sdAb)/VHH fusions, orthogonal Fab-Fab, scFv2-albumin/toxin fusions, single-chain diabody-albumin/toxin fusion, tandem scFv albumin/toxin fusions, dock-and-lock (DNL) Fab₃, DNL-Fab₂-scFv, DNL-Fab-IgG fusions, ImmTAC TCR-scFv fusions, IgG with different heavy chains and different or common light chains, IgG-scFv fusions to the heavy or light chain N or C terminus, IgG single-chain Fab (scFab) fusions to the heavy or light chain N- or C-terminus, single-chain IgG (scIgG) with scFv fusions, dual-variable domain (DVD) bispecific antibodies, asymmetric scFv-Fc, tandem-scFv-Fc fusions, dual affinity re-tarting antibodies (DART) with or without Fc fusion, asymmetric Fab-scFv-Fc fusions, scFv-CH3 fusions, TriFabs, IgG tandem scFv fusions, IgG-crossFab fusions, tandem Fab-IgG fusions with orthogonal Fabs, didiabody-Fc fusion, single-chain diabody Fc-fusion, Fab-scFv-Fc fusions, scFv₄-Fc fusion, scFv2-Fcab, Di-diabody, single-chain diabody CH3 fusion, IgE/M CH2 fusions, F(ab′)₂ fusions, CH1/CK fusions, two-in-one dual action Fabs (DAF), or DutaMab, DNL-Fab₂-IgG fusion.

Heterodimizeration of heavy chains for bispecific antibodies which contain Fc domains can be accomplished by a number of means including but not limited to knob-in-holes (Ridgway et al., Protein Eng (1996) 9(7):617-21; Atwell et al., J Mol Biol (1997) 270:26-35; Merchant et al., Nat Biotechnol (1998) 16:677-81), HA-TF mutations (Moore et al., Mabs (2011) 3:546-57), ZW1 (Von Kreudenstein et al., MAbs (2013) 5:646-54), C_{H}3 charge pairs (Gunasekaran et al., J Biol Chem (2010) 285:19637-46), IgG1 hinge/CH3 charge pairs (Strop et al., J Mol Biol (2012) 420:204-19), IgG2 hinge/CH3 charge pairs (Strop et al., J Mol Biol (2012) 420:204-19), EW-RVT mutations with or without an engineered disulfide (Choi et al., Mol Cancer Ther (2013) 12:2748-59; Choi et al., Mol Immunol (2015) 65:377-83), biclonic (Geuijen et al., J Clin Oncol (2014)), DuoBody (Labrijn et al., Proc Natl Acad Sci USA (2013) 110:5145-50), SEEDbody IgG/A chimera (Davis et al., Protein Eng Des Sel (2010) 23:195-202), BEAT (Moretti et al., BMC Proceedings (2013) 7, 09), 7.8.60 or 29.8.34 (Leaver-Fay et al., Structure (2016) 24:641-51). Correct pairing of different light chains can be accomplished by a variety of methods including, but not limited to CrossMab (Schaefer et al., Cancer Cell (2011) 20:472-86), orthologonal Fab (Lewis et al., Nat Biotechnol (2014) 32:191-8), T-cell receptor fusions (Wu et al., MAbs (2015) 7:470-82), CR3 (Golay et al., J Immunol (2016) 196:3199-211), MUT4 (Golay et al., J Immunol (2016) 196:3199-211), DuetMab (Mazor et al., MAbs (2015) 7:377-89; Mazor et al., MAbs (2015) 7:461-669).

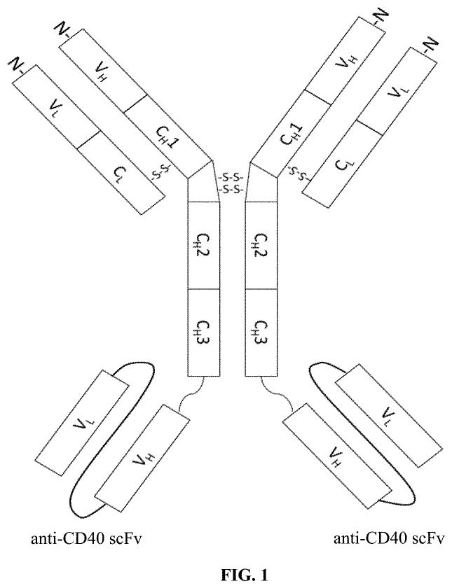

In certain embodiments, the anti-CD40 bispecific antibodies provided herein comprises two anti-CD40 scFv linked at each of the C-terminus of the heavy chain. In certain embodiments, the anti-CD40 scFc is linked to the heavy chain via a linker disclosed herein. In certain embodiments, the anti-CD40 bispecific antibodies provided herein have a structure as illustrated in FIG. 1.

### V. Conjugates

In some embodiments, the anti-CD40 antibodies and antigen-binding fragments thereof further comprise a conjugate moiety. The conjugate moiety can be linked to the antibodies and antigen-binding fragments thereof. A conjugate moiety is a non-proteinaceous moiety that can be attached to the antibody or antigen-binding fragment thereof. It is contemplated that a variety of conjugate moieties may be linked to the antibodies or antigen-binding fragments provided herein (see, for example, “Conjugate Vaccines”, Contributions to Microbiology and Immunology, J. M. Cruse and R. E. Lewis, Jr. (eds.), Carger Press, New York, (1989)). These conjugate moieties may be linked to the antibodies or antigen-binding fragments by covalent binding, affinity binding, intercalation, coordinate binding, complexation, association, blending, or addition, among other methods.

In certain embodiments, the antibodies and antigen-binding fragments disclosed herein may be engineered to contain specific sites outside the epitope binding portion that may be utilized for binding to one or more conjugate moieties. For example, such a site may include one or more reactive amino acid residues, such as for example cysteine or histidine residues, to facilitate covalent linkage to a conjugate moiety.

In certain embodiments, the antibodies may be linked to a conjugate moiety indirectly, or through another conjugate moiety. For example, the antibody or antigen-binding fragments may be conjugated to biotin, then indirectly conjugated to a second conjugate that is conjugated to avidin. The conjugate can be a clearance-modifying agent, a toxin (e.g., a chemotherapeutic agent), a detectable label (e.g., a radioactive isotope, a lanthanide, a luminescent label, a fluorescent label, or an enzyme-substrate label), or purification moiety.

A “toxin” can be any agent that is detrimental to cells or that can damage or kill cells. Examples of toxin include, without limitation, taxol, cytochalasin B, gramicidin D, ethidium bromide, emetine, mitomycin, etoposide, tenoposide, vincristine, MMAE, MMAF, DM1, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin and analogs thereof, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), anti-mitotic agents (e.g., vincristine and vinblastine), a topoisomerase inhibitor, and a tubulin-binders.

Examples of detectable label may include a fluorescent labels (e.g. fluorescein, rhodamine, dansyl, phycoerythrin, or Texas Red), enzyme-substrate labels (e.g. horseradish peroxidase, alkaline phosphatase, luceriferases, glucoamylase, lysozyme, saccharide oxidases or β-D-galactosidase), radioisotopes (e.g. ¹²³I, ¹²⁴I, ¹²⁵I, ¹³¹I, ³⁵S, ³H, ¹¹¹In, ¹¹²In, ¹⁴C, ⁶⁴Cu, ⁶⁷Cu, 86Y, 88 Y, ⁹⁰Y, ¹⁷⁷Lu, ²¹¹At, ¹⁸⁶Re, ¹⁸⁸Re, ¹⁵³Sm, ²¹²Bi, and ³²P, other lanthanides), luminescent labels, chromophoric moiety, digoxigenin, biotin/avidin, a DNA molecule or gold for detection.

In certain embodiments, the conjugate moiety can be a clearance-modifying agent which helps increase half-life of the antibody. Illustrative example include water-soluble polymers, such as PEG, carboxymethylcellulose, dextran, polyvinyl alcohol, polyvinyl pyrrolidone, copolymers of ethylene glycol/propylene glycol, and the like. The polymer may be of any molecular weight, and may be branched or unbranched. The number of polymers attached to the antibody may vary, and if more than one polymer are attached, they can be the same or different molecules.

In certain embodiments, the conjugate moiety can be a purification moiety such as a magnetic bead.

In certain embodiments, the antibodies and antigen-binding fragments thereof provided herein is used for a base for a conjugate.

### VI. Polynucleotides and Recombinant Methods

The present disclosure provides isolated polynucleotides that encode the anti-CD40 antibodies and antigen-binding fragments thereof. In certain embodiments, the isolated polynucleotides comprise one or more nucleotide sequences as shown in SEQ ID NOS: 9, 10, 19, 20, 29, 30, 39, 40, 49, 50, 59, 60, 69, 70, 79, 80, 89, 90, 99, 100, 109, 110, 119, 120, 129, 130, 139, 140, 149, 150, 159, 160, 169, 170, 179, 180, 189, 190, 199, 200, 209, 210, 219, 220, 229, 230, 239, 240, 249, 250, 259, 260, 269, 270, 279, 280, 289, 290, 299, 300, 309 and 310, which encodes the variable region of the exemplary antibodies provided herein. DNA encoding the monoclonal antibody is readily isolated and sequenced using conventional procedures (e.g., by using oligonucleotide probes that are capable of binding specifically to genes encoding the heavy and light chains of the antibody). The encoding DNA may also be obtained by synthetic methods.

The isolated polynucleotide that encodes the anti-CD40 antibodies and antigen-binding fragments thereof can be inserted into a vector for further cloning (amplification of the DNA) or for expression, using recombinant techniques known in the art. Many vectors are available. The vector components generally include, but are not limited to, one or more of the following: a signal sequence, an origin of replication, one or more marker genes, an enhancer element, a promoter (e.g. SV40, CMV, EF-1a), and a transcription termination sequence.

The present disclosure provides vectors (e.g., expression vectors) containing the nucleic acid sequence provided herein encoding the antibodies or antigen-binding fragments, at least one promoter (e.g., SV40, CMV, EF-1a) operably linked to the nucleic acid sequence, and at least one selection marker. Examples of vectors include, but are not limited to, retrovirus (including lentivirus), adenovirus, adeno-associated virus, herpesvirus (e.g., herpes simplex virus), poxvirus, baculovirus, papillomavirus, papovavirus (e.g., SV40), lambda phage, and M13 phage, plasmid pcDNA3.3, pMD18-T, pOptivec, pCMV, pEGFP, pIRES, pQD-Hyg-GSeu, pALTER, pBAD, pcDNA, pCal, pL, pET, pGEMEX, pGEX, pCI, pEGFT, pSV2, pFUSE, pVITRO, pVIVO, pMAL, pMONO, pSELECT, pUNO, pDUO, Psg5L, pBABE, pWPXL, pBI, p15TV-L, pPro18, pTD, pRS10, pLexA, pACT2.2, pCMV-SCRIPT®, pCDM8, pCDNA1.1/amp, pcDNA3.1, pRc/RSV, PCR 2.1, pEF-1, pFB, pSG5, pXT1, pCDEF3, pSVSPORT, pEF-Bos etc.

Vectors comprising the polynucleotide sequence encoding the antibody or antigen-binding fragment can be introduced to a host cell for cloning or gene expression. Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces.

In addition to prokaryotes, eukaryotic microbes such as filamentous fungi or yeast are suitable cloning or expression hosts for anti-CD40 antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; Yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated antibodies or antigen-fragment provided here are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TM cells (Mather et al., Annals N.Y. Acad. Sci. 383:44-68 (1982)); MRC 5 cells; FS4 cells; and a human hepatoma line (Hep G2). In some preferable embodiments, the host cell is 293F cell.

Host cells are transformed with the above-described expression or cloning vectors for anti-CD40 antibody production and cultured in conventional nutrient media modified as appropriate for inducing promoters, selecting transformants, or amplifying the genes encoding the desired sequences. In another embodiment, the antibody may be produced by homologous recombination known in the art.

The host cells used to produce the antibodies or antigen-binding fragments provided herein may be cultured in a variety of media. Commercially available media such as Ham's F10 (Sigma), Minimal Essential Medium (MEM), (Sigma), RPMI-1640 (Sigma), and Dulbecco's Modified Eagle's Medium (DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. No. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

When using recombinant techniques, the antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. Carter et al., Bio/Technology 10:163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The anti-CD40 antibodies prepared from the cells can be purified using, for example, hydroxylapatite chromatography, gel electrophoresis, dialysis, DEAE-cellulose ion exchange chromatography, ammonium sulfate precipitation, salting out, and affinity chromatography, with affinity chromatography being the preferred purification technique.

In certain embodiments, Protein A immobilized on a solid phase is used for immunoaffinity purification of the antibody and antigen-binding fragment thereof. The suitability of protein A as an affinity ligand depends on the species and isotype of any immunoglobulin Fc domain that is present in the antibody. Protein A can be used to purify antibodies that are based on human gamma1, gamma2, or gamma4 heavy chains (Lindmark et al., J. Immunol. Meth. 62:1-13 (1983)). Protein G is recommended for all mouse isotypes and for human gamma3 (Guss et al., EMBO J. 5:1567 1575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25M salt).

### VII. Pharmaceutical Composition

The present disclosure further provides pharmaceutical compositions comprising the anti-CD40 antibodies or antigen-binding fragments thereof and one or more pharmaceutically acceptable carriers.

Pharmaceutical acceptable carriers for use in the pharmaceutical compositions disclosed herein may include, for example, pharmaceutically acceptable liquid, gel, or solid carriers, aqueous vehicles, nonaqueous vehicles, antimicrobial agents, isotonic agents, buffers, antioxidants, anesthetics, suspending/dispending agents, sequestering or chelating agents, diluents, adjuvants, excipients, or non-toxic auxiliary substances, other components known in the art, or various combinations thereof.

Suitable components may include, for example, antioxidants, fillers, binders, disintegrants, buffers, preservatives, lubricants, flavorings, thickeners, coloring agents, emulsifiers or stabilizers such as sugars and cyclodextrins. Suitable antioxidants may include, for example, methionine, ascorbic acid, EDTA, sodium thiosulfate, platinum, catalase, citric acid, cysteine, thioglycerol, thioglycolic acid, thiosorbitol, butylated hydroxanisol, butylated hydroxytoluene, and/or propyl gallate. As disclosed herein, inclusion of one or more antioxidants such as methionine in a composition comprising an antibody or antigen-binding fragment and conjugates as provided herein decreases oxidation of the antibody or antigen-binding fragment. This reduction in oxidation prevents or reduces loss of binding affinity, thereby improving antibody stability and maximizing shelf-life. Therefore, in certain embodiments compositions are provided that comprise one or more antibodies or antigen-binding fragments as disclosed herein and one or more antioxidants such as methionine. Further provided are methods for preventing oxidation of, extending the shelf-life of, and/or improving the efficacy of an antibody or antigen-binding fragment as provided herein by mixing the antibody or antigen-binding fragment with one or more antioxidants such as methionine.

To further illustrate, pharmaceutical acceptable carriers may include, for example, aqueous vehicles such as sodium chloride injection, Ringer's injection, isotonic dextrose injection, sterile water injection, or dextrose and lactated Ringer's injection, nonaqueous vehicles such as fixed oils of vegetable origin, cottonseed oil, corn oil, sesame oil, or peanut oil, antimicrobial agents at bacteriostatic or fungistatic concentrations, isotonic agents such as sodium chloride or dextrose, buffers such as phosphate or citrate buffers, antioxidants such as sodium bisulfate, local anesthetics such as procaine hydrochloride, suspending and dispersing agents such as sodium carboxymethylcelluose, hydroxypropyl methylcellulose, or polyvinylpyrrolidone, emulsifying agents such as Polysorbate 80 (TWEEN-80), sequestering or chelating agents such as EDTA (ethylenediaminetetraacetic acid) or EGTA (ethylene glycol tetraacetic acid), ethyl alcohol, polyethylene glycol, propylene glycol, sodium hydroxide, hydrochloric acid, citric acid, or lactic acid. Antimicrobial agents utilized as carriers may be added to pharmaceutical compositions in multiple-dose containers that include phenols or cresols, mercurials, benzyl alcohol, chlorobutanol, methyl and propyl p-hydroxybenzoic acid esters, thimerosal, benzalkonium chloride and benzethonium chloride. Suitable excipients may include, for example, water, saline, dextrose, glycerol, or ethanol. Suitable non-toxic auxiliary substances may include, for example, wetting or emulsifying agents, pH buffering agents, stabilizers, solubility enhancers, or agents such as sodium acetate, sorbitan monolaurate, triethanolamine oleate, or cyclodextrin.

The pharmaceutical compositions can be a liquid solution, suspension, emulsion, pill, capsule, tablet, sustained release formulation, or powder. Oral formulations can include standard carriers such as pharmaceutical grades of mannitol, lactose, starch, magnesium stearate, polyvinyl pyrollidone, sodium saccharine, cellulose, magnesium carbonate, etc.

In certain embodiments, the pharmaceutical compositions are formulated into an injectable composition. The injectable pharmaceutical compositions may be prepared in any conventional form, such as for example liquid solution, suspension, emulsion, or solid forms suitable for generating liquid solution, suspension, or emulsion. Preparations for injection may include sterile and/or non-pyretic solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use, and sterile and/or non-pyretic emulsions. The solutions may be either aqueous or nonaqueous.

In certain embodiments, unit-dose parenteral preparations are packaged in an ampoule, a vial or a syringe with a needle. All preparations for parenteral administration should be sterile and not pyretic, as is known and practiced in the art.

In certain embodiments, a sterile, lyophilized powder is prepared by dissolving an antibody or antigen-binding fragment as disclosed herein in a suitable solvent. The solvent may contain an excipient which improves the stability or other pharmacological components of the powder or reconstituted solution, prepared from the powder. Excipients that may be used include, but are not limited to, water, dextrose, sorbital, fructose, corn syrup, xylitol, glycerin, glucose, sucrose or other suitable agent. The solvent may contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides a desirable formulation. In one embodiment, the resulting solution will be apportioned into vials for lyophilization. Each vial can contain a single dosage or multiple dosages of the anti-CD40 antibody or antigen-binding fragment thereof or composition thereof. Overfilling vials with a small amount above that needed for a dose or set of doses (e.g., about 10%) is acceptable so as to facilitate accurate sample withdrawal and accurate dosing. The lyophilized powder can be stored under appropriate conditions, such as at about 4° C. to room temperature.

Reconstitution of a lyophilized powder with water for injection provides a formulation for use in parenteral administration. In one embodiment, for reconstitution the sterile and/or non-pyretic water or other liquid suitable carrier is added to lyophilized powder. The precise amount depends upon the selected therapy being given, and can be empirically determined.

### VIII. Methods of Use

The present disclosure also provides therapeutic methods comprising: administering a therapeutically effective amount of the antibody or antigen-binding fragment as provided herein to a subject in need thereof, thereby treating or preventing a CD40-related condition or a disorder. In some embodiment, the CD40-related condition or a disorder is cancer, autoimmune disease, inflammatory disease, or infectious disease.

Examples of cancer include but are not limited to, non-small cell lung cancer (squamous/nonsquamous), small cell lung cancer, renal cell cancer, colorectal cancer, colon cancer, ovarian cancer, breast cancer (including basal breast carcinoma, ductal carcinoma and lobular breast carcinoma), pancreatic cancer, gastric carcinoma, bladder cancer, esophageal cancer, mesothelioma, melanoma, head and neck cancer, thyroid cancer, sarcoma, prostate cancer, glioblastoma, cervical cancer, thymic carcinoma, melanoma, myelomas, mycoses fungoids, merkel cell cancer, hepatocellular carcinoma (HCC), fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, lymphoid malignancy, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, hepatoma, bile duct carcinoma, choriocarcinoma, Wilms' tumor, cervical cancer, testicular tumor, seminoma, classical Hodgkin lymphoma (CHL), primary mediastinal large B-cell lymphoma, T-cell/histiocyte-rich B-cell lymphoma, acute lymphocytic leukemia, acute myelocytic leukemia, acute myelogenous leukemia, chronic myelocytic (granulocytic) leukemia, chronic myelogenous leukemia, chronic lymphocytic leukemia, polycythemia vera, mast cell derived tumors, EBV-positive and -negative PTLD, and diffuse large B-cell lymphoma (DLBCL), plasmablastic lymphoma, extranodal NK/T-cell lymphoma, nasopharyngeal carcinoma, HHV8-associated primary effusion lymphoma, non-Hodgkin's lymphoma, multiple myeloma, Waldenstrom's macroglobulinemia, heavy chain disease, myelodysplastic syndrome, hairy cell leukemia and myelodysplasia, primary CNS lymphoma, spinal axis tumor, brain stem glioma, astrocytoma, medulloblastoma, craniopharyogioma, ependymoma, pinealoma, hemangioblastoma, acoustic neuroma, oligodendroglioma, menangioma, melanoma, neuroblastoma and retinoblastoma.

Autoimmune diseases include, but are not limited to, Acquired Immunodeficiency Syndrome (AIDS, which is a viral disease with an autoimmune component), alopecia areata, ankylosing spondylitis, antiphospholipid syndrome, autoimmune Addison's disease, autoimmune hemolytic anemia, autoimmune hepatitis, autoimmune inner ear disease (AIED), autoimmune lymphoproliferative syndrome (ALPS), autoimmune thrombocytopenic purpura (ATP), Behcet's disease, cardiomyopathy, celiac sprue-dermatitis hepetiformis; chronic fatigue immune dysfunction syndrome (CFIDS), chronic inflammatory demyelinating polyneuropathy (CIPD), cicatricial pemphigold, cold agglutinin disease, crest syndrome, Crohn's disease, Degos' disease, dermatomyositis-juvenile, discoid lupus, essential mixed cryoglobulinemia, fibromyalgia-fibromyositis, Graves' disease, Guillain-Barre syndrome, Hashimoto's thyroiditis, idiopathic pulmonary fibrosis, idiopathic thrombocytopenia purpura (ITP), IgA nephropathy, insulin-dependent diabetes mellitus, juvenile chronic arthritis (Still's disease), juvenile rheumatoid arthritis, Meniere's disease, mixed connective tissue disease, multiple sclerosis, myasthenia gravis, pemacious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomena, Reiter's syndrome, rheumatic fever, rheumatoid arthritis, sarcoidosis, scleroderma (progressive systemic sclerosis (PSS), also known as systemic sclerosis (SS)), Sjogren's syndrome, stiff-man syndrome, systemic lupus erythematosus, Takayasu arteritis, temporal arteritis/giant cell arteritis, ulcerative colitis, uveitis, vitiligo and Wegener's granulomatosis. Inflammatory disorders, include, for example, chronic and acute inflammatory disorders. Examples of inflammatory disorders include Alzheimer's disease, asthma, atopic allergy, allergy, atherosclerosis, bronchial asthma, eczema, glomerulonephritis, graft vs. host disease, hemolytic anemias, osteoarthritis, sepsis, stroke, transplantation of tissue and organs, vasculitis, diabetic retinopathy and ventilator induced lung injury. In some embodiments, the CD3 associated conditions are inflammatory diseases such as systemic lupus erythematosus (SLE), intestinal mucosal inflammation, wasting disease associated with colitis, multiple sclerosis, viral infections, rheumatoid arthritis, osteoarthritis, Cohn's disease, and inflammatory bowel disease, psoriasis, systemic scleroderma, autoimmune diabetes and the like.

Infectious disease include, but are not limited to, fungus infection, parasite/protozoan infection or chronic viral infection, for example, malaria, coccidioiodmycosis immitis, histoplasmosis, onychomycosis, aspergilosis, blastomycosis, candidiasis albicans, paracoccidioiomycosis, microsporidiosis, Acanthamoeba keratitis, Amoebiasis, Ascariasis, Babesiosis, Balantidiasis, Baylisascariasis, Chagas disease, Clonorchiasis, Cochliomyia, Cryptosporidiosis, Diphyllobothriasis, Dracunculiasis, Echinococcosis, Elephantiasis, Enterobiasis, Fascioliasis, Fasciolopsiasis, Filariasis, Giardiasis, Gnathostomiasis, Hymenolepiasis, Isosporiasis, Katayama fever, Leishmaniasis, Lyme disease, Metagonimiasis, Myiasis, Onchocerciasis, Pediculosis, Scabies, Schistosomiasis, Sleeping sickness, Strongyloidiasis, Taeniasis, Toxocariasis, Toxoplasmosis, Trichinosis, Trichuriasis, Trypanosomiasis, helminth infection, infection of hepatitis B (HBV), hepatitis C (HCV), herpes virus, Epstein-Barr virus, HIV, cytomegalovirus, herpes simplex virus type I, herpes simplex virus type II, human papilloma virus, adenovirus, human immunodeficiency virus I, human immunodeficiency virus II, Kaposi West sarcoma associated herpes virus epidemics, thin ring virus (Torquetenovirus), human T lymphotrophic viruse I, human T lymphotrophic viruse II, varicella zoster, JC virus or BK virus.

The therapeutically effective amount of an antibody or antigen-binding fragment as provided herein will depend on various factors known in the art, such as for example body weight, age, past medical history, present medications, state of health of the subject and potential for cross-reaction, allergies, sensitivities and adverse side-effects, as well as the administration route and extent of disease development. Dosages may be proportionally reduced or increased by one of ordinary skill in the art (e.g., physician or veterinarian) as indicated by these and other circumstances or requirements.

In certain embodiments, the antibody or antigen-binding fragment as provided herein may be administered at a therapeutically effective dosage of about 0.01 mg/kg to about 100 mg/kg. In certain of these embodiments, the antibody or antigen-binding fragment is administered at a dosage of about 50 mg/kg or less, and in certain of these embodiments the dosage is 10 mg/kg or less, 5 mg/kg or less, 3 mg/kg or less, 1 mg/kg or less, 0.5 mg/kg or less, or 0.1 mg/kg or less. In certain embodiments, the administration dosage may change over the course of treatment. For example, in certain embodiments the initial administration dosage may be higher than subsequent administration dosages. In certain embodiments, the administration dosage may vary over the course of treatment depending on the reaction of the subject.

Dosage regimens may be adjusted to provide the optimum desired response (e.g., a therapeutic response). For example, a single dose may be administered, or several divided doses may be administered over time.

The antibodies and antigen-binding fragments disclosed herein may be administered by any route known in the art, such as for example parenteral (e.g., subcutaneous, intraperitoneal, intravenous, including intravenous infusion, intramuscular, or intradermal injection) or non-parenteral (e.g., oral, intranasal, intraocular, sublingual, rectal, or topical) routes.

In some embodiments, the antibodies or antigen-binding fragments disclosed herein may be administered alone or in combination with one or more additional therapeutic means or agents. For example, the antibodies or antigen-binding fragments disclosed herein may be administered in combination with another therapeutic agent, for example, a chemotherapeutic agent or an anti-cancer drug.

In certain of these embodiments, an antibody or antigen-binding fragment as disclosed herein that is administered in combination with one or more additional therapeutic agents may be administered simultaneously with the one or more additional therapeutic agents, and in certain of these embodiments the antibody or antigen-binding fragment and the additional therapeutic agent(s) may be administered as part of the same pharmaceutical composition. However, an antibody or antigen-binding fragment administered “in combination” with another therapeutic agent does not have to be administered simultaneously with or in the same composition as the agent. An antibody or antigen-binding fragment administered prior to or after another agent is considered to be administered “in combination” with that agent as the phrase is used herein, even if the antibody or antigen-binding fragment and second agent are administered via different routes. Where possible, additional therapeutic agents administered in combination with the antibodies or antigen-binding fragments disclosed herein are administered according to the schedule listed in the product information sheet of the additional therapeutic agent, or according to the Physicians' Desk Reference 2003 (Physicians' Desk Reference, 57th Ed; Medical Economics Company; ISBN: 1563634457; 57th edition (November 2002)) or protocols well known in the art.

The present disclosure further provides methods of using the anti-CD40 antibodies or antigen-binding fragments thereof.

In some embodiments, the present disclosure provides methods of detecting presence or amount of CD40 in a sample, comprising contacting the sample with the antibody or antigen-binding fragment thereof, and determining the presence or the amount of CD40 in the sample.

In some embodiments, the present disclosure provides methods of diagnosing a CD40 related disease or condition in a subject, comprising: a) contacting a sample obtained from the subject with the antibody or antigen-binding fragment thereof provided herein; b) determining presence or amount of CD40 in the sample; and c) correlating the existence of the CD40 to the CD40 related disease or condition in the subject.

In some embodiments, the present disclosure provides kits comprising the antibody or antigen-binding fragment thereof provided herein, optionally conjugated with a detectable moiety. The kits may be useful in detection of CD40 or diagnosis of CD40 related disease.

In some embodiments, the present disclosure also provides use of the antibody or antigen-binding fragment thereof provided herein in the manufacture of a medicament for treating a CD40 related disease or condition in a subject, in the manufacture of a diagnostic reagent for diagnosing a CD40 related disease or condition.

The following examples are provided to better illustrate the claimed invention and are not to be interpreted as limiting the scope of the invention. All specific compositions, materials, and methods described below, in whole or in part, fall within the scope of the present invention. These specific compositions, materials, and methods are not intended to limit the invention, but merely to illustrate specific embodiments falling within the scope of the invention. One skilled in the art may develop equivalent compositions, materials, and methods without the exercise of inventive capacity and without departing from the scope of the invention. It will be understood that many variations can be made in the procedures herein described while still remaining within the bounds of the present invention. It is the intention of the inventors that such variations are included within the scope of the invention.

### Example 1: Materials and Methods

Reagents

CP-870,893, a IgG2 isotype fully human anti-CD40 mAb was purified from a HEK293 cell line and produced at Revmab (Lot R181219-1). APX005m, a humanized anti-CD40 mAb, IgG1 isotype with S267E mutation, was purified form a HEK293 cell line and produced at Revmab (Lot R190724-2). 3B5, a humanized anti-CD40 mAb, IgG1 isotype, was purified from a HEK293 cell line and produced at Revmab (Lot R200429-1). Human IgG1 isotype control was purchased from Bioxell (Catalog BE 0297, Lot 760619N1).

3B5ScFv-PD-L1 (or PDL1-3B5 scFv), a tetravalent bispecific antibody, comprises an anti-programmed death-ligand 1 (PD-L1) antibody, and two single-chain variable fragments (scFv) that are genetically fused to the carboxyl termini of the heavy chain. The human version of 3B5ScFv-PD-L1 uses anti-human-PD-L1 antibody of IgG4 isotype. The mouse version of 3B5ScFv-PD-L1 uses anti-mouse PD-L1, clone 10F.9G2, IgG2a isotype with D265A and N297A mutations (Lot #R201007-3).

R10 media contains Roswell Park Memorial Institute Medium (RPMI) 1640 with L-glutamine (Corning, Catalog 10-040-CM) supplemented with 10% Fetal Bovine Serum (FBS, Sigma, Catalog F4135). D10 media contains Dulbecco's Modification of Eagle's Medium (DMEM) with 4.5 g/L glucose, L-glutamine, sodium pyruvate (Corning, Catalog 10-013-CM) supplemented with 10% Fetal Bovine Serum (FBS, Sigma, Catalog F4135). Classical Monocyte Isolation Kit for human was purchased from Miltenyi Biotec (Catalog 130-117-337). Recombinant Human Granulocyte-Macrophage Colony Stimulating Factor (GM-CSF) was purchased from Peprotech (Catalog 300-03). Recombinant Human Interleukin 4 (IL-4) was purchased from Peprotech (Catalog 200-04). FACS Buffer contains Phosphate-Buffered Saline (PBS, Sigma Catalog D8537) supplied with 2 mM Ethylenediaminetetraacetic acid (EDTA, EMT Millipore Catalog 324506) and 2% Fetal Bovine Serum (FBS, Sigma, Catalog F4135)

Antibodies and reagents used for flow cytometry staining are listed in Table 3

TABLE 3
Antibodies and Reagents

| Marker | Fluorophore | Clone | Vendor | Catalog No. |
|---|---|---|---|---|
| CD80 | BV421 | 2D10 | BioLegend | 305222 |
| CD86 | APC | IT2.2 | BioLegend | 305412 |
| CD11c | FITC | 3.9 | BioLegend | 301604 |
| Mouse IgG | Alexa Fluor | polyclone | Jackson | 715-605-150 |
| (H + L) | 647 | | ImmunoResearch | |
| Human IgG | Alexa Fluor | polyclone | Jackson | 709-605-149 |
| (H + L) | 647 | | ImmunoResearch | |
| Propidium | | | BioLegend | 421301 |
| Iodide (PI) | | | | |
| DAPI | | | BioLegend | 422801 |

Cells

HEK-PDL1, a human embryonic kidney (HEK) cell line engineered to stably express mouse PD-L1, was made by Apollomics (Lot APL-2-PDL1-01). HEK-CD40, a HEK cell line engineered to stably express human CD40 and NFκB-luciferase reporter with puromycin and green fluorescent protein (GFP) dual selection markers, was made by Apollomics (Lot APL-1-CD40RE-03). Ramos-Blue™ cell, engineered to stably express an NF-κB/AP-1-inducible SEAP (secreted embryonic alkaline phosphatase) reporter gene, was purchased from InvivoGen (Catalog rms-sp). Ramos cell was purchased from ATCC (Catalog CRL-1596).

Methods

ELISA Assay

ELISA plate was coated with purified recombinant CD40 protein in coating buffer at lug/ml overnight. After washed for 3 times, the plate was blocked with 1% BSA, and then cell culture supernatants containing rabbit-human chimeric CD40 antibodies were added to the plate. For detection of the antibody binding to CD40 protein, goat anti-human Fc polyclonal antibody conjugated to alkaline phosphatase was used.

Binding to Cell Surface Human CD40 in Flow Cytometry Experiments

HEK-CD40 were collected and plated at 10⁵ cells per well in 96-well round bottom plate in FACS buffer. Serial dilutions of antibodies (Control IgG1, CP870,893, APX005m and 3B5) in 100 ul were incubated with the HEK-CD40 cells for 60 min at 4° C. Cells were washed 3 times with 200 ul FACS buffer. Cell-bound antibodies were detected using an APC-conjugated secondary antibody specific to human IgG heavy and light chain. After incubating 30 min with secondary antibody, HEK-CD40 cells were washed 3 times with 200 ul FACS buffer and resuspended in 200 ul FACS with 1 ug/ml DAPI. Flow cytometric analyses were performed using Attune N×T flow cytometer (ThermoFisher Scientific, Waltham, Mass.). After gating on FCS-SSC parameters to exclude debris and selectively gating DAPI negative cells on DAPI-SSC parameters to exclude dead cells, the geometric mean fluorescence intensity (MFI) of antibodies was determined using FlowJo v10 (BD Bioscience, San Jose, Calif.). Dose-response curves were generated and EC50s calculated using GraphPad Prism 8 (GraphPad Software, San Diego, Calif.).

Stimulation of Ramos-Blue Cells

Ramos-Blue cells were counted and resuspended in fresh R10 medium at a density of 4×10⁶ cells/ml. 400,000 cells per well were added in a flat-bottom 96-well plate. HEK-Control cells or HEK-PD-L1 cells which engineered express mouse PD-L1, were added at 60,000 cells per well for a final Ramos to HEK ratio of 20:3. A serial dilution of antibodies (CP870,893, APX005m and PDL1-3B5 scFv) was added to each well and the plate was then incubated at 37° C. in a 5% CO₂ incubator for 18 h.

QUANTI-Blue™ Assay

QUANTI-Blue™ solution was prepared by adding 1 ml of QB reagent and 1 ml of QB buffer to 98 ml of sterile water and mixed well by vertexing and incubate at room temperature for 10 minutes. Immediately before use, 180 ul QUANTI-Blue™ solution and 20 ul cell supernatants from Ramos-blue cells were mixed at 10:1 ratio and added per well in a flat-bottom 96-well plate. The assay plate was incubated at 37° C. for 1 h and read on SpectraMax ID3 (Molecular Devices, San Jose, Calif.) at OD 630.

Differentiation and Activation of Monocyte-Derived Human Dendritic Cells (moDCs)

Frozen peripheral blood mononuclear cells (PBMCs) were purchased from Physician's Plasma Alliance Research Group (Johnson City, Tenn.). Monocyte were purified from PBMCs using a magnetic bead-based separation kit. Purified monocytes were resuspended at 2×10⁶ cells/ml in R10 media supplemented with 100 ng/ml IL-4 and 100 ng/ml GM-CSF and plated at 2×10⁶ cells/well into a 6 well plate. Half of the medium were replaced every two days. Immature DCs were differentiated from monocytes after 4 days by incubating at 37° C. in a 5% CO₂ incubator.

In vitro moDCs were lifted and resuspend in fresh R10 medium at a density of 1×10⁶ cells/ml. 100,000 cells per well were added in a flat-bottom 96-well plate. HEK-Control cells or HEK-PDL1 cells which engineered express mouse PDL1, were added at 20,000 cells per well for a final moDC to HEK ratio of 5:1. A serial dilution of antibodies (CP870,893, APX005m and PDL1-3B5 scFv) was added to each well and the plate was then incubated at 37° C. in a 5% CO₂ incubator. After 48 hours incubation, moDCs were collected by pipetting and washed with FACS buffer and resuspended in 200 ul FACS buffer with 5% human serum for blocking. After 30 minutes in blocking buffer, moDCs were stained with anti-CD11c, anti-CD80, anti-CD86 antibodies and viability dye. Flow cytometric analyses were performed using MAC SQuant Analyzer 10 (Milentyi, Germany).

### Example 2: Identification of Anti-CD40 Antibodies

A group of rabbit anti-CD40 antibodies were generated by immunization of rabbits with human CD40 antigen. Anti-CD40 rabbit hybridoma clones were produced and used to determine the anti-CD40 variable region sequences. The variable region sequences were fused to human Fc region to generate rabbit-human chimeric antibodies, which were assayed for their binding to human CD40 using ELISA. The results of the ELISA are shown in Table 4 below.

TABLE 4
Binding of Anti-CD40 Antibodies to Recombinant Human CD40

| Clone | OD₄₀₅ | Clone | OD₄₀₅ | Clone | OD₄₀₅ |
|---|---|---|---|---|---|
| 1D3 | 1.51 | 6G8 | 1.77 | 2E11 | 1.79 |
| 2B8 | 1.64 | 6H9 | 1.97 | 5F9 | 1.83 |
| 3B12 | 1.57 | 7A6 | 1.99 | 5D10 | 0.98 |
| 3B5 | 1.48 | 7C11 | 1.8 | 192 | 2 |
| 2E3 | 1.62 | 8C10 | 2.09 | 194 | 1.69 |
| 4A7 | 1.65 | 2C10 | 2.14 | 195 | 1.84 |
| 3E12 | 1.39 | 7G6 | 1.82 | 196 | 1.92 |
| 4B7 | 1.72 | 7H11 | 1.46 | 197 | 2.01 |
| 4F11 | 1.54 | 8C6 | 1.97 | 198 | 1.8 |
| 6C2 | 1.96 | 7D2 | 1.82 | No Ab | 0.07 |
| 6E1 | 1.82 | 7F10 | 1.9 | | |

### Example 3: Binding to Cell Surface Human CD40

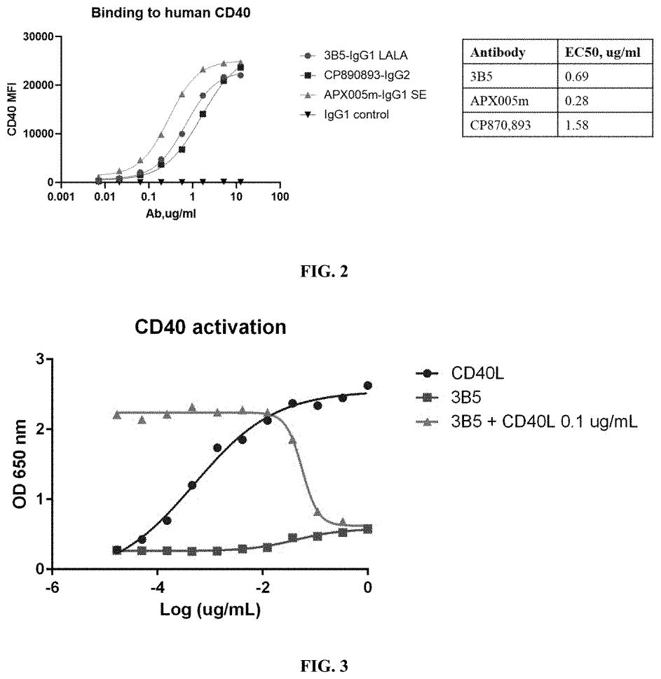

In a flow cytometry experiment, the titration of CP870,893, APX005m and 3B5, but not control IgG1, on HEK-CD40 cells showed binding to cell surface human CD40. EC50 calculated based on MFI data from the experiments are summarized in FIG. 2 and Table 5.

TABLE 5
MFI of antibodies binding to cell surface CD40

| | mAb Concentration [μg/ml] | | | | | | | |
|---|---|---|---|---|---|---|---|---|
| Antibody | 12 | 5 | 1.7 | 0.56 | 0.78 | 0.06 | 0.02 | 0.007 |
| CP770, 793 | 23682 | 20923 | 14092 | 6792 | 3668 | 1533 | 635 | 271 |
| APX005m | 24712 | 24538 | 23319 | 18153 | 9840 | 4591 | 2364 | 1147 |
| 3B5 | 22050 | 21693 | 17875 | 9989 | 4747 | 2088 | 847 | 363 |
| IgG control | 73.7 | 112 | 61.3 | 30.7 | 32.3 | 40.2 | 40.1 | 28.3 |

### Example 3: Competing with CD40L

The competition between 3B5 and CD40L leads to the decreased CD40 activation by CD40L. Briefly, HEKBlue CD40L cells were treated with CD40L, 3B5 separately or in combination. As shown in FIG. 3, 3B5 blocks CD40L induced CD40 activation in a dose dependent manner.

### Example 4: Low Basal Activity

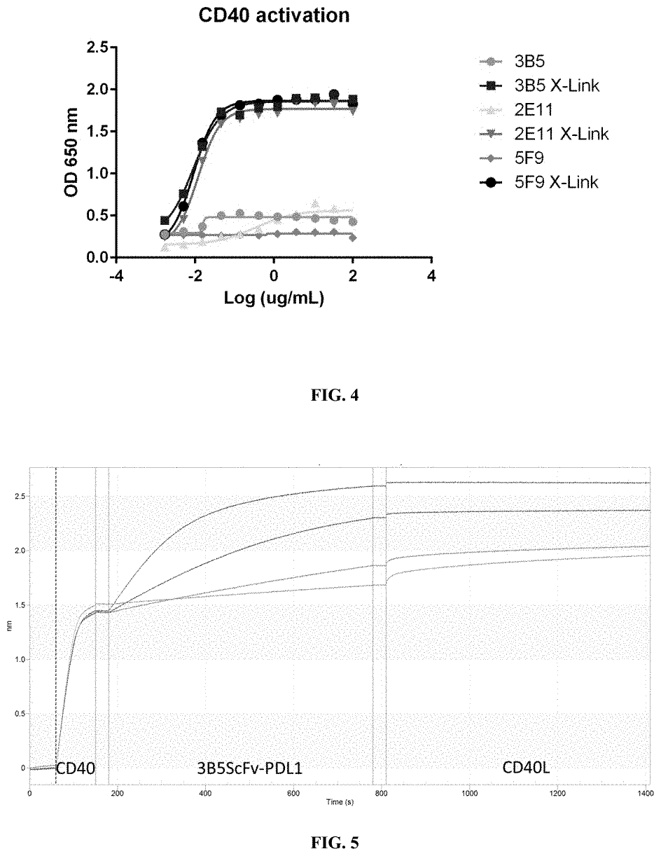

HEKBlue CD40L cells were used to test the ability of candidate antibodies to activate CD40 signaling with or without crosslinking. Briefly, 5×10⁴ HEKBlue cells were treated with the anti-CD40 antibody with or without the presence of Fab2 fragment targeting human IgG Fc that crosslinks the anti-CD40 antibody. The CD40 activity was measured by detecting SEAP level in the supernatant. As shown in FIG. 4, anti-CD40 antibodies 3B5, 2E11 and 5F9 showed low basal activity of inducing CD40 activation in the absence of crosslinking and activated CD40 after the anti-CD40 antibodies are crosslinked by the Fab2 fragment targeting human IgG Fc.

### Example 5: Generating Anti-CD40 Bispecific Antibody

Two version of anti-CD40 bispecific antibodies were generated based on clone 3B5: 1) 3B5ScFv PD-L1, wherein 3B5 ScFv is fused to the C-terminus of each heavy chain of a PD-L1 antibody; and 2) PD-L1 ScFc 3B5, wherein PD-L1 ScFc is fused to the C-terminus of each heavy chain of 3B5 antibody. The anti-CD40 bispecific antibodies were then tested for their binding to CD40 using Octet. As shown in Table 6, 3B5ScFv_PD-L1 retains the high affinity of 3B5 while PD-L1 ScFc_3B5 deceases the affinity to CD40 compared to 3B5.

TABLE 6
Binding affinity to recombinant CD40

| | KD (M) | kon(1/Ms) | kdis(1/s) |
|---|---|---|---|
| 3B5 | <1.0E−12 | 1.06E+06 | <1.0E−07 |
| 3B5ScFv_PD-L1 | 1.30E−11 | 7.78E+05 | 1.01E−05 |
| PD-L1ScFv_ 3B5 | <1.0E−12 | 1.56E+05 | <1.0E−07 |

### Example 6: Bispecific Antibody Competing with CD40L Binding

Competition between 3B5ScFv_PD-L1 and CD40L on CD40 binding were measured using Octet. Briefly, recombinant CD40 were loaded onto the probe and subsequently exposed to 3B5ScFv_PDL1 and CD40L. As shown in FIG. 5, decreasing CD40L binding were observed with increasing 3B5ScFv_PDL1 concentration.

### Example 7: Activation of Ramos-Blue Cells

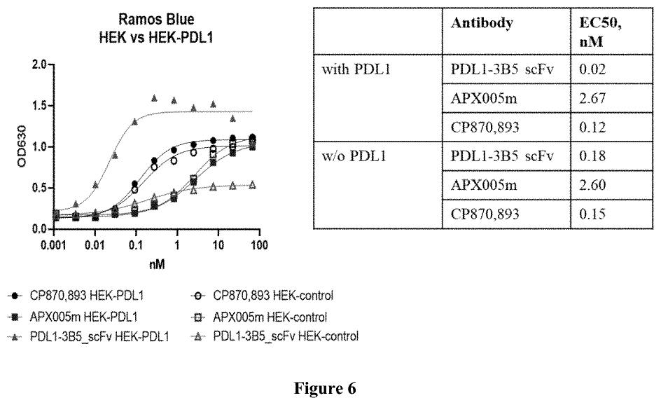

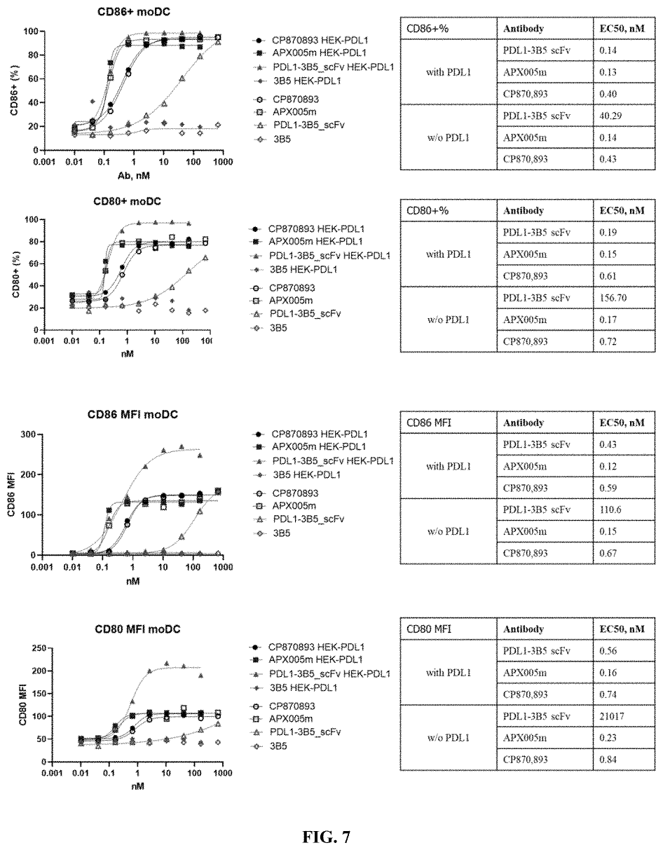

The stimulation of Ramos-Blue cells was assessed by measuring the NFkB-SEAP reporter activity using QUANTI-Blue™ assay. As shown in FIG. 6, PDL1-3B5 scFv showed dose-dependent activation Ramos-Blue cells, only in the presence of HEK-PDL1 cells, suggesting that the crosslink-mediated CD40 agonistic activity by 3B5 is relying on the bispecific binding of PD-L1. Consistently, two control antibodies, CP870,893 and APX005m, whose crosslink activities are independent of PD-L1 expression, showed equal activation of Ramos-Blue cells in the present or absent of HEK-PD-L1 cells.

### Example 8: Activation of Human Primary moDC

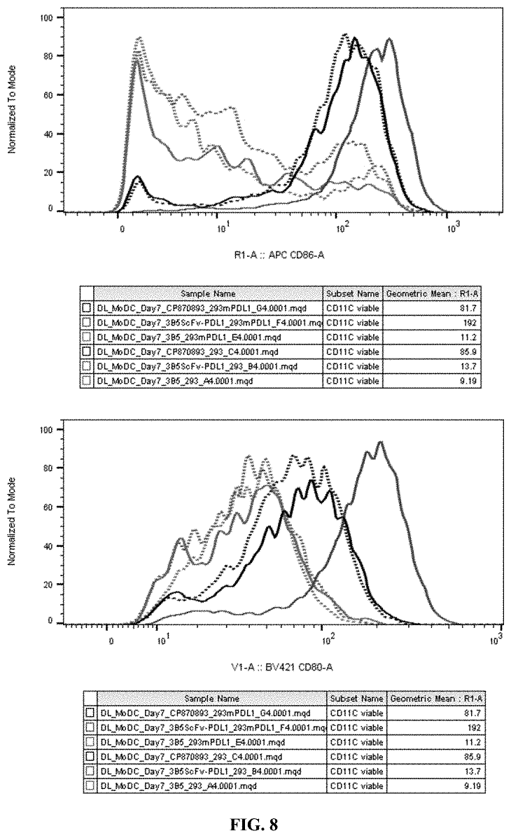

In vitro derived human moDCs were analyzed by gating for CD11c positive and PI negative viable dendritic cells. The activation of moDCs was assessed by surface expression of co-stimulatory molecules CD80 and CD86 using flow cytometry, as well as the percentage of CD80 or CD86 positive cells. As shown in FIG. 7, 3B5 antibody did not promote differentiation of monocyte into dendritic cells nor enhance the expression of the activation marker CD80 and CD86. The bispecific PDL1-3B5 scFv significantly enhanced DC differentiation and activation in the presence of PD-L1 comparing to the absence of PD-L1. In addition, as shown in FIG. 8, the expression level of CD80 and CD86 were much higher on cells treated with bispecific PDL1-3B5 scFv. At the same concentration, PDL1-3B5 scFv leads to much stronger activation of DCs comparing to antibody CP87,893.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. An isolated anti-CD40 antibody or an antigen-binding fragment thereof, comprising:
a) a heavy chain variable region comprising
heavy chain complementarity determining region (HCDR)1 of amino acid sequence TYAX₁S (SEQ ID NO: 313), wherein X₁ is M or V,
HCDR2 of amino acid sequence IIDX₂X₃X₄X₅X₆X₇YAX₈WX₉KG (SEQ ID NO: 314, wherein each of X₂, X₃ and X₄ is independently G or S, X₅ is F or G, X₆ is A or T, X₇ is A or V, X₈ is N or S, X₉ is A or V, and
HCDR3 of amino acid sequence GX₁₀TFFX₁₁L (SEQ ID NO: 315), wherein X₁₀ is A or V, and X₁₁ is G or N; and
b) a light chain variable region comprising
light chain complementarity determining region (LCDR)1 of amino acid sequence QASQSISX₁₂X₁₃LX₁₄ (SEQ ID NO: 316), wherein X₁₂ is G, N or S, X₁₃ is A, V or Y, and X₁₄ is A or S,
LCDR2 of amino acid sequence RAX₁₅X₁₆LX₁₇S (SEQ ID NO: 317), wherein each of X₁₅ is A or S, X₁₆ is D or T, X₁₇ is A or E, and
LCDR3 of amino acid sequence QX₁₈YYYSX₁₉X₂₀GSYX₂₁YA (SEQ ID NO: 318), wherein X₁₈ is A or S, X₁₉ is G, N or S, X₂₀ is G or S, and X₂₁ is D or S.

2. The antibody or an antigen-binding fragment thereof of claim 1, wherein
a) the heavy chain variable region comprises HCDR1 of SEQ ID NO: 31, 41 or 51, HCDR2 of SEQ ID NO: 32, 42 or 52, and HCDR3 of SEQ ID NO: 33, 43 and 53; and
b) the light chain variable region comprises LCDR1 of SEQ ID NO: 34, 44 or 54, LCDR2 of SEQ ID NO: 35, 45 or 55, and LCDR3 of SEQ ID NO: 36, 46 or 56.

3. The antibody or an antigen-binding fragment thereof of claim 1, wherein
(a) the heavy chain variable region comprises HCDR1 of SEQ ID NO: 31, HCDR2 of SEQ ID NO: 32, and HCDR3 of SEQ ID NO: 33; and the light chain variable region comprises LCDR1 of SEQ ID NO: 34, LCDR2 of SEQ ID NO: 35, and LCDR3 of SEQ ID NO: 36; or
(b) the heavy chain variable region comprises HCDR1 of SEQ ID NO: 41, HCDR2 of SEQ ID NO: 42, and HCDR3 of SEQ ID NO: 43; and the light chain variable region comprises LCDR1 of SEQ ID NO: 44, LCDR2 of SEQ ID NO: 45, and LCDR3 of SEQ ID NO: 46; or
(c) the heavy chain variable region comprises HCDR1 of SEQ ID NO: 51, HCDR2 of SEQ ID NO: 52, and HCDR3 of SEQ ID NO: 53; and the light chain variable region comprises LCDR1 of SEQ ID NO: 54, LCDR2 of SEQ ID NO: 55, and LCDR3 of SEQ ID NO: 56.

4. The antibody or an antigen-binding fragment thereof of claim 1, wherein
(a) the heavy chain variable region has an amino acid sequence at least about 90% identical to SEQ ID NO: 37; and the light chain variable region has an amino acid sequence at least about 90% identical to SEQ ID NO: 38; or
(b) the heavy chain variable region has an amino acid sequence at least about 90% identical to SEQ ID NO: 47; and the light chain variable region has an amino acid sequence at least about 90% identical to SEQ ID NO: 48; or
(c) the heavy chain variable region has an amino acid sequence at least about 90% identical to SEQ ID NO: 57; and the light chain variable region has an amino acid sequence at least about 90% identical to SEQ ID NO: 58.

5. The antibody or an antigen-binding fragment thereof of claim 1, wherein
(a) the heavy chain variable region has an amino acid sequence of SEQ ID NO: 37; and
the light chain variable region has an amino acid sequence of SEQ ID NO: 38; or
(b) the heavy chain variable region has an amino acid sequence of SEQ ID NO: 47; and
the light chain variable region has an amino acid sequence of SEQ ID NO: 48; or
(c) the heavy chain variable region has an amino acid sequence of SEQ ID NO: 57; and
the light chain variable region has an amino acid sequence of SEQ ID NO: 58.

6. The antibody or antigen-binding fragment thereof of claim 1, wherein the antibody competes with CD40L to bind to CD40.

7. The antibody or antigen-binding fragment thereof of claim 6, wherein the antibody is capable of selectively activating CD40 in tumor microenvironment.

8. The antibody or antigen-binding fragment thereof of claim 7, wherein the antibody is capable of activating dendritic cells.

9. The antibody or an antigen-binding fragment thereof of claim 1, which is chimeric or humanized.

10. The antibody or an antigen-binding fragment thereof of claim 9, wherein the heavy chain variable region has an amino acid sequence at least about 90% identical to SEQ ID NO: 311; and the light chain variable region has an amino acid sequence at least about 90% identical to SEQ ID NO: 312.

11. The antibody or antigen-binding fragment thereof of claim 1, which is a scFv (single chain fragment variable), a Fab fragment, a Fab′ fragment, a F(ab′)₂ fragment, or a Fv fragment.

12. The antibody or antigen-binding fragment thereof of claim 1, which is a bispecific antibody.

13. The antibody or an antigen-binding fragment thereof of claim 12, which specifically binds to an antigen enriched in tumor microenvironment.

14. The antibody or an antigen-binding fragment thereof of claim 13, wherein the antigen is selected from (CEA), CA-125, MUC-1, epithelial tumor antigen (ETA), tyrosinase, and melanoma-associated antigen (MAGE).

15. The antibody or an antigen-binding fragment thereof of claim 12, which specifically binds to an immune checkpoint.

16. The antibody or an antigen-binding fragment thereof of claim 15, wherein the immune checkpoint is selected from A2AR, B7.1, B7.2, B7-H2, B7-H3, B7-H4, B7-H6, BTLA, CD48, CD160, CD244, CTLA-4, ICOS, LAG-3, LILRB1, LILRB2, LILRB4, OX40, PD-1, PD-L1, PD-L2, SIRPalpha (CD47), TIGIT, TIM-3, TIM-1, TIM-4, and VISTA.

17. The antibody or an antigen-binding fragment thereof of claim 12, comprising a scFv.

18. The antibody or an antigen-binding fragment thereof of claim 17, wherein the scFv has an amino acid sequence of SEQ ID NO: 319.

19. A pharmaceutical composition comprising the antibody or antigen-binding fragment thereof of claim 1, and a pharmaceutically acceptable carrier.

20. An isolated polynucleotide encoding the antibody or an antigen-binding fragment thereof of claim 1.

21. A vector comprising the isolated polynucleotide of claim 20.

22. A host cell comprising the vector of claim 21.

23. A method of producing an anti-CD40 antibody or antigen-binding fragment thereof, comprising culturing the host cell of claim 22 under the condition at which the anti-CD40 antibody or antigen-binding fragment thereof is expressed.

24. A method of treating a disease or condition in a subject that would benefit from modulation of CD40 activity, comprising administering to the subject a therapeutically effective amount of the antibody or antigen-binding fragment thereof of claim 1.

25. The method of claim 24, wherein the disease or condition is a CD40 related disease or condition.

26. The method of claim 25, wherein the disease or condition is cancer.

27. The method of claim 26, wherein the cancer is adrenal cancer, bone cancer, brain cancer, breast cancer, colorectal cancer, esophageal cancer, eye cancer, gastric cancer, head and neck cancer, kidney cancer, liver cancer, lung cancer, non-small cell lung cancer, bronchioloalveolar cell lung cancer, mesothelioma, head and neck cancer, squamous cell carcinoma, lymphoma, lymphocytic leukemia, melanoma, oral cancer, ovarian cancer, cervical cancer, penile cancer, prostate cancer, pancreatic cancer, skin cancer, sarcoma, testicular cancer, thyroid cancer, uterine cancer, vaginal cancer, and Hodgkin's Disease.

28. The method of claim 24, wherein the subject is human.

29. The method of claim 24, wherein the administration is via oral, nasal, intravenous, subcutaneous, sublingual, or intramuscular administration.


SEQUENCE LISTING (319 sequences)

SEQ ID NO: 1 - protein, 5 aa, Rattus rattus
NYHMC

SEQ ID NO: 2 - protein, 16 aa, Rattus rattus
YNSYGGSAYYASWAKG

SEQ ID NO: 3 - protein, 13 aa, Rattus rattus
AYLDYNGDMGFAL

SEQ ID NO: 4 - protein, 11 aa, Rattus rattus
QASQSISSYLS

SEQ ID NO: 5 - protein, 7 aa, Rattus rattus
RASTLES

SEQ ID NO: 6 - protein, 13 aa, Rattus rattus
QTNYYDSGNTYGT

SEQ ID NO: 7 - protein, 118 aa, Rattus rattus
QSLEESGGRLVTPGTPLTLTCTASGFSLSNYHMCWVRQAPGKGLEWIGYNSYGGSAYYASWAKGRFTISKTSTTVDLKITRPTTEDTATYFCARAYLDYNGDMGFALWGPGTLVTVSS

SEQ ID NO: 8 - protein, 112 aa, Rattus rattus
AEVVMTQTPSSVSAAVGGTVTFKCQASQSISSYLSWYQQKPGQPPKLLIYRASTLESGVPSRFKGSGSGTQFTLTISDLECADAATYYCQTNYYDSGNTYGTFGGGTEVVVK

SEQ ID NO: 9 - DNA, 354 bp, Rattus rattus
cagtcgctggaggagtccgggggtcgcctggtcacgcctgggacacccctgacactcacctgcacagcctctggattctccctcagtaactaccacatgtgctgggtccgccaggctccagggaaggggctggagtggatcggatacaatagttatggtggtagcgcatactacgcgagctgggcgaaaggccgattcaccatctccaaaacctcgaccacggtggatctgaaaatcaccagaccgacaaccgaggacacggccacctatttctgtgccagagcatatcttgattataatggtgatatgggttttgccttgtggggcccaggcaccctggtcaccgtctcctca

SEQ ID NO: 10 - DNA, 336 bp, Rattus rattus
gccgaagtagtgatgacccagactccatcctccgtgtctgcagctgtgggaggcacagtcaccttcaagtgccaggccagtcagagcattagcagttacttatcctggtatcagcagaaaccagggcagcctcccaagctcctgatctacagggcatccactctggaatctggggtcccatcgcggttcaaaggcagtggatctgggacacagttcactctcaccatcagcgacctggagtgtgccgatgctgccacttactactgtcaaaccaactattatgatagtggtaatacttatggtactttcggcggagggaccgaggtggtggtcaaa

SEQ ID NO: 11 - protein, 5 aa, Rattus rattus
SYAMG

SEQ ID NO: 12 - protein, 17 aa, Rattus rattus
VIASNSGISYYATWAEG

SEQ ID NO: 13 - protein, 8 aa, Rattus rattus
GYIGDGAL

SEQ ID NO: 14 - protein, 13 aa, Rattus rattus
QSSKSVYYNNWLA

SEQ ID NO: 15 - protein, 7 aa, Rattus rattus
RASTLAS

SEQ ID NO: 16 - protein, 11 aa, Rattus rattus
AGGYSSDSDTT

SEQ ID NO: 17 - protein, 114 aa, Rattus rattus
QSLEESGGRLVTPGTPLTLTCTVSGIDLSSYAMGWVRQAPGKGLEYIGVIASNSGISYYATWAEGRFTISKASTTVDLKITSPTTEDTATYFCVRGYIGDGALWGPGTLVTVSS

SEQ ID NO: 18 - protein, 110 aa, Rattus rattus
AVLTQTPSSVSAAVGGTVNIGCQSSKSVYYNNWLAWYQQKPGQPPKLLIYRASTLASGVPSRFKGSGSGTQFTLTISDVQCADATTYYCAGGYSSDSDTTFGGGTEVVVK

SEQ ID NO: 19 - DNA, 342 bp, Rattus rattus
cagtcgctggaggagtccgggggtcgcctggtcacgcctgggacacccctgacactcacctgcacagtctctggaatcgacctcagtagttatgcaatgggctgggtccgccaggctccagggaaggggctggaatacatcggagtcattgctagtaatagtggtatctcatactacgcgacctgggcagaaggccgattcaccatctccaaggcctcgaccacggtggatctgaaaatcaccagtccgacaaccgaagacacggccacctatttctgtgtcagaggatatattggtgatggtgccttgtggggcccaggcaccctggtcaccgtctcctca

SEQ ID NO: 20 - DNA, 330 bp, Rattus rattus
gccgtgctgacccagactccatcctccgtgtctgcagctgtgggaggcacagtcaacatcggttgccagtccagtaagagtgtttattacaacaactggttagcctggtatcagcagaaaccagggcagcctcccaagctcctaatctacagggcatccactctggcatctggggtcccatcgcggttcaaaggcagtggatctgggacacagttcactctcaccatcagcgacgtgcagtgtgccgatgctaccacttactattgtgcaggcggttatagtagtgatagtgatactactttcggcggagggaccgaggtggtggtcaaa

SEQ ID NO: 21 - protein, 5 aa, Rattus rattus
SYAMG

SEQ ID NO: 22 - protein, 17 aa, Rattus rattus
VIASNSGISYYATWAEG

SEQ ID NO: 23 - protein, 8 aa, Rattus rattus
GYIGDGAL

SEQ ID NO: 24 - protein, 13 aa, Rattus rattus
QSSKTVYYNNWLA

SEQ ID NO: 25 - protein, 7 aa, Rattus rattus
RASTLAS

SEQ ID NO: 26 - protein, 11 aa, Rattus rattus
AGGFSSDSDTT

SEQ ID NO: 27 - protein, 114 aa, Rattus rattus; identical to SEQ ID NO: 17

SEQ ID NO: 28 - protein, 110 aa, Rattus rattus
AVLTQTPSSVSAAVGGTVTINCQSSKTVYYNNWLAWYQQKPGQPPKLLIYRASTLASGVPSRFKGSGSGTQFTLTISDVQCDDATTYYCAGGFSSDSDTTFGGGTEVVVK

SEQ ID NO: 29 - DNA, 342 bp, Rattus rattus
cagtcgctggaggagtccgggggtcgcctggtcacgcctgggacacccctgacactcacctgcacggtctctggaatcgacctcagtagttatgcaatgggctgggtccgccaggctccagggaaggggctggaatacatcggagtcattgctagtaatagtggtatctcatactacgcgacctgggcagaaggccgattcaccatctccaaggcctcgaccacggtggatctgaaaatcaccagtccgacaaccgaagacacggccacctatttctgtgtcagaggatatattggtgatggtgccttgtggggcccaggcaccctggtcaccgtctcctca

SEQ ID NO: 30 - DNA, 330 bp, Rattus rattus
gccgtgctgacccagactccatcctccgtgtctgcagctgtgggaggcacagtcaccatcaattgccagtccagtaagactgtttattataataactggttagcctggtatcagcagaaaccagggcagcctcccaagctcctaatctacagggcatccactctggcatctggggtcccatcgcggttcaaaggcagtggatctgggacacagttcactctcaccatcagcgacgtgcagtgtgacgatgctaccacttactattgtgcaggcggtttcagtagtgatagtgatactactttcggcggagggaccgaggtggtggtcaaa

SEQ ID NO: 31 - protein, 5 aa, Rattus rattus
TYAVS

SEQ ID NO: 32 - protein, 16 aa, Rattus rattus
IIDSGGGTAYASWVKG

SEQ ID NO: 33 - protein, 7 aa, Rattus rattus
GVTFFGL

SEQ ID NO: 34 - protein, 11 aa, Rattus rattus
QASQSISNYLS

SEQ ID NO: 35 - protein, 7 aa, Rattus rattus
RASDLAS

SEQ ID NO: 36 - protein, 14 aa, Rattus rattus
QAYYYSGGGSYDYA

SEQ ID NO: 37 - protein, 112 aa, Rattus rattus
QSVEESGGRLITPGTPLTLTCTVSGFSLSTYAVSWVRQAPGKGLEWIGIIDSGGGTAYASWVKGRFTISKTSTTVDLKITSPTTEDTATYFCARGVTFFGLWGPGTLVTVSS

SEQ ID NO: 38 - protein, 113 aa, Rattus rattus
ADIVMTQTPASVEAAVGGTVTIKCQASQSISNYLSWYQQKPGQPPKLLIYRASDLASGVPSRFKGSGSGTEFTLTISDLECADAATYYCQAYYYSGGGSYDYAFGGGTEVVVK

SEQ ID NO: 39 - DNA, 336 bp, Rattus rattus
cagtcggtggaggagtccgggggtcgcctaatcacgcctgggacaccccttacactcacctgcacagtctctggattctccctcagtacctatgcagtgagctgggtccgccaggctccagggaaggggctggaatggatcggaatcattgatagtggtggtggcaccgcctacgcgagctgggtgaaaggccgattcaccatctccaaaacctcgaccacggtagatctcaaaatcaccagtccgacaaccgaggacacggccacctatttctgtgccagaggagttaccttctttggcttgtggggcccaggcaccctggtcaccgtctcctca

SEQ ID NO: 40 - DNA, 339 bp, Rattus rattus
gctgacattgtgatgacccagactccagcctccgtggaggcagctgtgggaggcacagtcaccatcaagtgccaggccagtcagagcattagtaattacttatcctggtatcagcagaaaccagggcagcctcccaagctcctgatctacagggcatccgatctggcatctggggtcccatcgcggttcaaaggcagtggatctgggacagagttcactctcaccatcagcgacctggagtgtgccgatgctgccacttactactgtcaagcctattattatagtggtggtggtagttatgattacgctttcggcggagggaccgaggtggtggtcaaa

SEQ ID NO: 41 - protein, 5 aa, Rattus rattus
TYAMS

SEQ ID NO: 42 - protein, 16 aa, Rattus rattus
IIDGSSFTVYANWAKG

SEQ ID NO: 43 - protein, 7 aa, Rattus rattus
GATFFGL

SEQ ID NO: 44 - protein, 11 aa, Rattus rattus
QASQSISGVLA

SEQ ID NO: 45 - protein, 7 aa, Rattus rattus
RAADLAS

SEQ ID NO: 46 - protein, 14 aa, Rattus rattus
QSYYYSNSGSYSYA

SEQ ID NO: 47 - protein, 112 aa, Rattus rattus
QSVEESGGRLVTPGTPLTLTCTVSGFSLSTYAMSWVRQAPGKGLEWIGIIDGSSFTVYANWAKGRFTISKTSTTVDLKMTSLTASDTATYFCARGATFFGLWGPGTLVTVSS

SEQ ID NO: 48 - protein, 113 aa, Rattus rattus
ADVVMTQTPASVSAAVGGTVTIKCQASQSISGVLAWYQQKPGQPPKLLIYRAADLASGVPSRFKGSGSGTEFTLTINDLECDDAATYYCQSYYYSNSGSYSYAFGGGTEVVVK

SEQ ID NO: 49 - DNA, 336 bp, Rattus rattus
cagtcggtggaggagtccgggggtcgcctggtcacgcctgggacacccctgacactcacctgcacagtctctggattctccctcagtacctatgcaatgagctgggtccgccaggctccagggaaggggctggaatggatcggaatcattgatggtagttcttttacagtctacgcgaactgggcgaaaggccgattcaccatctccaaaacctcgaccacggtggatctgaaaatgaccagtctgacagcttcagacacggccacctatttctgtgccagaggagctaccttctttggcttgtggggcccaggcaccctggtcaccgtctcctca

SEQ ID NO: 50 - DNA, 339 bp, Rattus rattus
gccgacgtcgtgatgacccagactccagcctccgtgtctgcagctgtgggaggcacagtcaccatcaagtgccaggccagtcagagcattagtggcgtcttagcctggtatcagcagaaaccagggcagccccccaagctcctgatctacagggcagccgatctggcatctggggtcccatcgcggttcaaaggcagtggatctgggacagagttcactctcaccatcaacgacctggagtgtgacgatgctgccacttactactgtcaaagctattattatagtaatagtggtagttatagttacgctttcggcggagggaccgaggtggtggtcaaa

SEQ ID NO: 51 - protein, 5 aa, Rattus rattus
TYAMS

SEQ ID NO: 52 - protein, 16 aa, Rattus rattus
IIDGGGFAVYANWAKG

SEQ ID NO: 53 - protein, 7 aa, Rattus rattus
GVTFFNL

SEQ ID NO: 54 - protein, 11 aa, Rattus rattus
QASQSISSALA

SEQ ID NO: 55 - protein, 7 aa, Rattus rattus
RASTLES

SEQ ID NO: 56 - protein, 14 aa, Rattus rattus
QSYYYSSGGSYDYA

SEQ ID NO: 57 - protein, 112 aa, Rattus rattus
QSVEESGGRLVTPGTPLTLTCTVSGFSLSTYAMSWVRQAPGKGLEWIGIIDGGGFAVYANWAKGRFTISKASTTVDLKLPSPTTEDTATYFCARGVTFFNLWGPGTLVTVSS

SEQ ID NO: 58 - protein, 113 aa, Rattus rattus
ADIVMTQTPASVEAAVGDTVTIKCQASQSISSALAWYQQKPGQPPKLLIYRASTLESGVPSRFKGSGSGTEFTLTISDLECADAATYYCQSYYYSSGGSYDYAFGGGTEVVVK

SEQ ID NO: 59 - DNA, 336 bp, Rattus rattus
cagtcggtggaggagtccgggggtcgcctggtcacgcctgggacacccctgacactcacctgcacagtctctggattctccctcagtacctatgcaatgagctgggtccgccaggctccagggaaggggctggaatggatcggaatcattgatggtggtggtttcgcagtctacgcgaactgggcgaaaggccgattcaccatctccaaagcctcgaccacggtggatctgaaactccccagtccgacaaccgaggacacggccacctatttctgtgccagaggagttaccttctttaacttgtggggcccaggcaccctggtcaccgtctcctca

SEQ ID NO: 60 - DNA, 339 bp, Rattus rattus
gctgacattgtgatgacccagactccagcctccgtggaggcagctgtgggagacacagtcaccatcaagtgccaggccagtcagagcattagtagcgccttagcctggtatcagcagaaaccagggcagcctcccaagctcctgatctacagggcatccactctggaatctggggtcccatcgcggttcaaaggcagtggatctgggacagagttcactctcaccatcagcgacctggagtgtgccgatgctgccacttactactgtcaaagttattattatagtagtggtggtagttatgattacgctttcggcggagggaccgaggtggtggtcaaa

SEQ ID NO: 61 - protein, 5 aa, Rattus rattus
NYRMI

SEQ ID NO: 62 - protein, 16 aa, Rattus rattus
VIDRNDNTYYATWAKS

SEQ ID NO: 63 - protein, 9 aa, Rattus rattus
DTDYTTGGI

SEQ ID NO: 64 - protein, 11 aa, Rattus rattus
QASQNIGDNLA

SEQ ID NO: 65 - protein, 7 aa, Rattus rattus
GASTLAS

SEQ ID NO: 66 - protein, 14 aa, Rattus rattus
QSNYYSTSSSYYNS

SEQ ID NO: 67 - protein, 116 aa, Rattus rattus
QSVKESEGGLFKPTDTLTLTCTVSGFSFSNYRMIWVRQAPGNGLEWIGVIDRNDNTYYATWAKSRSTITRNTNLNTVTLKMTSLTAADTATYFCARDTDYTTGGIWGPGTLVTVSS

SEQ ID NO: 68 - protein, 113 aa, Rattus rattus
ADIVMTQTPASVSAAVGGTVTIKCQASQNIGDNLAWHQQKPGQPPKLLIYGASTLASGVPSRFKGSGSRTEYTLTISDLECADAATYYCQSNYYSTSSSYYNSFGGGTEVVVK

SEQ ID NO: 69 - DNA, 348 bp, Rattus rattus
cagtcggtgaaggagtccgagggaggtctcttcaagccaacggataccctgacactcacctgcacagtctctggattctccttcagtaattatcgaatgatctgggtccgccaggctccagggaacgggctggaatggatcggagtcattgatagaaatgataacacatactacgcgacctgggcgaagagccgatccaccatcaccagaaacaccaacctgaacacggtgactctgaaaatgaccagtctgacagccgcggacacggccacctatttctgtgcgagagacactgattatactactggtggcatctggggcccaggcaccctggtcaccgtctcctca

SEQ ID NO: 70 - DNA, 339 bp, Rattus rattus
gctgacattgtgatgacccagactccagcctccgtgtctgcagctgtgggaggcacagtcaccatcaagtgccaggccagtcagaacattggtgataatttagcctggcatcagcagaaaccagggcagcctcccaagctcctgatctatggtgcatccactctggcttctggggtcccatcgcggttcaaaggcagtggatctaggacagagtacactctcaccatcagcgacctggagtgtgccgatgctgccacttactactgtcaaagtaattattatagtactagtagtagttattataattccttcggcggagggaccgaggtggtggtcaaa

SEQ ID NO: 71 - protein, 5 aa, Rattus rattus
NYRVI

SEQ ID NO: 72 - protein, 16 aa, Rattus rattus
GIDRSAVAYYASWAKS

SEQ ID NO: 73 - protein, 9 aa, Rattus rattus
DTDYTSGGI

SEQ ID NO: 74 - protein, 11 aa, Rattus rattus
QASQNIGGNLA

SEQ ID NO: 75 - protein, 7 aa, Rattus rattus
GSSTLES

SEQ ID NO: 76 - protein, 14 aa, Rattus rattus
QSNYWSRSSSYVNS

SEQ ID NO: 77 - protein, 116 aa, Rattus rattus
QSVKESEGGLFKPTDTLTLTCTVSGFTLSNYRVIWVRQAPGNGLEWIGGIDRSAVAYYASWAKSRSTITRNTNLNTVTLKMTSLTAADTATYFCARDTDYTSGGIWGPGTLVTVSS

SEQ ID NO: 78 - protein, 112 aa, Rattus rattus
DIVMTQTPSSVSAAVGGTVTIKCQASQNIGGNLAWYQQKPGQPPKLLIYGSSTLESGVPSRFSGSGSGTEYTLTISDLQCADAATYYCQSNYWSRSSSYVNSFGGGTEVVVK

SEQ ID NO: 79 - DNA, 348 bp, Rattus rattus
cagtcggtgaaggagtccgagggaggtctcttcaagccaacggataccctgacactcacctgcacagtctctggattcaccctcagtaactatcgagtgatctgggtccgccaggctccagggaacgggctggaatggatcggaggcattgataggagtgctgtcgcatattacgcgagctgggcgaaaagccggtccaccatcaccagaaacaccaacctgaacacggtgactctgaaaatgaccagtctgacggccgcggacacggccacctatttctgtgcgagagacactgattatactagtggtggcatctggggcccaggcaccctggtcaccgtctcctca

SEQ ID NO: 80 - DNA, 336 bp, Rattus rattus
gacattgtgatgacccagactccatcctccgtgtctgcagctgtgggaggcacagtcaccatcaagtgccaggccagtcagaacattggtggtaatttagcctggtatcagcagaaaccagggcagcctcccaagctcctgatctatggttcatccactctggaatctggggtcccatcgcggttcagcggcagtggatctgggacagagtacactctcaccatcagcgacctgcagtgtgccgatgctgccacttactactgtcaaagtaattattggagtcgtagtagtagttatgttaattctttcggcggagggaccgaggtggtggtcaaa

SEQ ID NO: 81 - protein, 5 aa, Rattus rattus
SNALS

SEQ ID NO: 82 - protein, 16 aa, Rattus rattus
IINSYGSTYYASWAKG

SEQ ID NO: 83 - protein, 17 aa, Rattus rattus
VLEYSSTDYGDWGYFNL

SEQ ID NO: 84 - protein, 11 aa, Rattus rattus
QASEDIASNLA

SEQ ID NO: 85 - protein, 7 aa, Rattus rattus
GASNLES

SEQ ID NO: 86 - protein, 12 aa, Rattus rattus
QTSYYDSNTRNV

SEQ ID NO: 87 - protein, 122 aa, Rattus rattus
QSLEESGGRLVTPGTPLTLTRTVSGFSLSSNALSWVRQAPGKGLEWIGIINSYGSTYYASWAKGRFTISKTSTTVDLRITSPTPEDTATYFCARVLEYSSTDYGDWGYFNLWGPGTLVTVSS

SEQ ID NO: 88 - protein, 110 aa, Rattus rattus
AYDMTQTPSPVSAAVGGTVTINCQASEDIASNLAWYQQKPGQRPKLLIYGASNLESGVPSRFKGSGSGTEYTLTISDLECADAATYYCQTSYYDSNTRNVFGGGTEVVVK

SEQ ID NO: 89 - DNA, 366 bp, Rattus rattus
cagtcgctggaggagtccgggggtcgcctggtcacgcctgggacacccctgacactcacccgcaccgtctctggattctccctcagtagcaatgcattaagctgggtccgccaggctccagggaaggggctggaatggatcggaatcattaatagttatggtagcacatactacgcgagctgggcgaaaggccgattcaccatctccaaaacctcgaccacggtagatctgcgaatcaccagtccgacacccgaggacacggccacctatttctgtgccagagtattggaatatagcagtactgactacggtgattggggttactttaacttgtggggcccaggcaccctggtcaccgtctcctca

SEQ ID NO: 90 - DNA, 330 bp, Rattus rattus
gcctatgacatgacccagactccatcccccgtgtctgcagctgtgggaggcacagtcaccatcaattgccaggccagtgaggatattgctagtaatttagcctggtatcagcagaaaccagggcagcgtcccaagctcctgatctatggtgcatccaatctggaatctggggtcccatcgcggttcaaaggcagtggatctgggacagagtacactctcaccatcagcgacctggagtgtgccgatgctgccacttactattgtcaaacttcttattatgatagtaatactcggaatgttttcggcggagggaccgaggtggtggtcaaa

SEQ ID NO: 91 - protein, 5 aa, Rattus rattus
TYYMI

SEQ ID NO: 92 - protein, 16 aa, Rattus rattus
IISSNGNAYYATWAKG

SEQ ID NO: 93 - protein, 12 aa, Rattus rattus
AASIANSYYFNV

SEQ ID NO: 94 - protein, 13 aa, Rattus rattus
QSSQSVRDNNWLS

SEQ ID NO: 95 - protein, 7 aa, Rattus rattus
GASNLES

SEQ ID NO: 96 - protein, 11 aa, Rattus rattus
QGGFSTYSDNS

SEQ ID NO: 97 - protein, 117 aa, Rattus rattus
QSVEESGGRLVTPGTPLTLTCTASGFSLSTYYMIWVRQAPGKGLDYIGIISSNGNAYYATWAKGRFTISKTSTTVDLKMTSPTTEDTATYFCARAASIANSYYFNVWGPGTLVTVSS

SEQ ID NO: 98 - protein, 110 aa, Rattus rattus
AVLTQTPSPVSAAVGGTVSISCQSSQSVRDNNWLSWFQQKPGHPPKLLIYGASNLESGVPSRFSGSGSGTEFTLTISDVQCDDAATYYCQGGFSTYSDNSFGGGTEVVVK

SEQ ID NO: 99 - DNA, 351 bp, Rattus rattus
cagtcggtggaggagtccgggggtcgcctggtcacgcctgggacacccctgacactcacctgcacagcctctggattctccctcagtacctactacatgatctgggtccgccaggctccaggaaaggggctggactacatcggaataattagtagtaatggtaacgcatactacgcgacctgggcaaaaggccgattcaccatctccaaaacctcgaccacggtggatctgaaaatgaccagtccgacaaccgaggacacggccacctatttctgtgccagagcggcttctattgctaattcctactactttaatgtctggggcccaggcaccctggtcaccgtctcctca

SEQ ID NO: 100 - DNA, 330 bp, Rattus rattus
gccgtgctgacccagactccatctcccgtgtctgcagctgtgggaggcacagtcagcatcagttgccagtccagtcagagtgttcgtgataacaactggttatcctggtttcagcagaaaccagggcaccctcccaagctcctgatctatggtgcatccaatctggaatctggggtcccatcgcggttcagcggcagtggatctgggacagagttcactctcaccatcagcgacgtgcagtgtgacgatgctgccacttactactgtcaaggcggttttagtacttatagtgataatagtttcggcggagggaccgaggtggtggtcaaa

SEQ ID NO: 101 - protein, 5 aa, Rattus rattus
SYAMT

SEQ ID NO: 102 - protein, 16 aa, Rattus rattus
IIDTIGSTYYASWAKG

SEQ ID NO: 103 - protein, 11 aa, Rattus rattus
DYIGSTSGKKL

SEQ ID NO: 104 - protein, 13 aa, Rattus rattus
QSSKSVYYKNWLA

SEQ ID NO: 105 - protein, 7 aa, Rattus rattus
GASTLAS

SEQ ID NO: 106 - protein, 11 aa, Rattus rattus
AGGYSSDSDTG

SEQ ID NO: 107 - protein, 116 aa, Rattus rattus
QSVEESGGRLVTPGTPLTLTCTVSGFSLSSYAMTWVRQAPGKGLEWLGIIDTIGSTYYASWAKGRFTISKTSTTVDLKITSPTTEDTATYFCVRDYIGSTSGKKLWGRGTLVTVSS

SEQ ID NO: 108 - protein, 110 aa, Rattus rattus
AVLTQTPSSVEAAVGGTVSISCQSSKSVYYKNWLAWYQQKPGQPPKLLIYGASTLASGVPSRFKGSGSGAQFTLTISDVQCDDAATYYCAGGYSSDSDTGFGGGTEVVVK

SEQ ID NO: 109 - DNA, 348 bp, Rattus rattus
cagtcggtggaggagtccgggggtcgcctggtcacgcctgggacacccctgacactcacctgcacagtctctggattctccctcagtagctatgcaatgacctgggtccgccaggctccagggaaggggctggaatggctcggaatcattgatactattggtagcacatactacgcgagctgggcgaaaggccgattcaccatctccaagacctcgaccacggtggatctgaaaatcaccagtccgacaaccgaggacacggccacctatttctgtgtcagagactatattggtagtacttcgggaaaaaagttgtggggccgaggcaccctggtcaccgtctcctca

SEQ ID NO: 110 - DNA, 330 bp, Rattus rattus
gccgtgctgacccagactccatcctccgtggaggcagctgtgggaggcacagtcagcatcagttgccagtccagtaagagtgtttattataaaaactggttagcctggtatcagcagaaaccagggcagcctcccaaactcctgatctatggtgcatccactctggcatctggggtcccatcgcgattcaaaggcagtggatctggggcacagttcactctcaccatcagcgacgtgcagtgtgacgatgctgccacttactattgtgcaggcggttatagtagtgatagtgatactggtttcggcggagggaccgaggtggtggtcaaa

SEQ ID NO: 111 - protein, 5 aa, Rattus rattus
TYYMT

SEQ ID NO: 112 - protein, 16 aa, Rattus rattus
YINTGGSTYYASRAKG

SEQ ID NO: 113 - protein, 13 aa, Rattus rattus
ADASEHNYNGLNI

SEQ ID NO: 114 - protein, 11 aa, Rattus rattus
QASESISSNLA

SEQ ID NO: 115 - protein, 7 aa, Rattus rattus
RASTLAS

SEQ ID NO: 116 - protein, 14 aa, Rattus rattus
QSNYGSISSSYGNA

SEQ ID NO: 117 - protein, 118 aa, Rattus rattus
QSVEESGGRLVTPGTPLTLTCTASGFSLSTYYMTWVRQAPGKGLEAIGYINTGGSTYYASRAKGRFTISKTSTTVDLKMTSPTTEDTATYFCARADASEHNYNGLNIWGPGTLVTVSS

SEQ ID NO: 118 - protein, 113 aa, Rattus rattus
ADIVMTQTPASVSEPVGGTVTIKCQASESISSNLAWYQQKPGQPPKFLIYRASTLASGVPSRFKGSGFGTQFTLTISDLECADAATYYCQSNYGSISSSYGNAFGGGTEVVVK

SEQ ID NO: 119 - DNA, 354 bp, Rattus rattus
cagtcggtggaggagtccgggggtcgcctggtcacgcctgggacacccctgacactcacctgcacagcctctggattctccctcagtacctactacatgacctgggtccgccaggctccagggaaggggctggaggccatcggatacattaatactggtggtagcacatactacgcgagccgggcgaaaggccgattcaccatctccaaaacctcgaccacggtggatctgaaaatgaccagtccgacaaccgaggacacggccacctatttctgtgccagagctgatgctagtgagcataactataatggccttaacatctggggcccaggcaccctggtcaccgtctcctca

SEQ ID NO: 120 - DNA, 339 bp, Rattus rattus
gctgacattgtgatgacccagactccagcctccgtgtctgaacctgtgggaggcacagtcaccatcaagtgccaggccagtgagagcattagtagcaatttagcctggtatcagcagaaaccagggcagcctcccaagttcctgatctacagggcatccactctggcatctggggtcccatcgcggttcaaaggcagtggatttgggacacagttcactctcaccatcagcgacctggagtgtgccgatgctgccacttactactgtcaaagcaattatggtagtattagtagtagttatggtaatgctttcggcggagggaccgaggtggtggtcaaa

SEQ ID NO: 121 - protein, 5 aa, Rattus rattus
SYAMG

SEQ ID NO: 122 - protein, 17 aa, Rattus rattus
VIASNSGISYYATWAEG

SEQ ID NO: 123 - protein, 8 aa, Rattus rattus
GYIGDGAL

SEQ ID NO: 124 - protein, 13 aa, Rattus rattus
QSSKTVYYNNWLA

SEQ ID NO: 125 - protein, 7 aa, Rattus rattus
RASTLAS

SEQ ID NO: 126 - protein, 11 aa, Rattus rattus
AGGFSSDSDTT

SEQ ID NO: 127 - protein, 114 aa, Rattus rattus; identical to SEQ ID NO: 17

SEQ ID NO: 128 - protein, 110 aa, Rattus rattus; identical to SEQ ID NO: 28

SEQ ID NO: 129 - DNA, 342 bp, Rattus rattus; identical to SEQ ID NO: 19

SEQ ID NO: 130 - DNA, 330 bp, Rattus rattus; identical to SEQ ID NO: 30

SEQ ID NO: 131 - protein, 5 aa, Rattus rattus
TWTMS

SEQ ID NO: 132 - protein, 16 aa, Rattus rattus
IINSDSNTYYASWAKG

SEQ ID NO: 133 - protein, 14 aa, Rattus rattus
GVGGYSSGPDYLDI

SEQ ID NO: 134 - protein, 11 aa, Rattus rattus
QASQSIYRSLA

SEQ ID NO: 135 - protein, 7 aa, Rattus rattus
HASDLAS

SEQ ID NO: 136 - protein, 14 aa, Rattus rattus
QSYYYSGINTYGNA

SEQ ID NO: 137 - protein, 119 aa, Rattus rattus
QSVEESGGRLVTPGTPLTLACTVSGIDLSTWTMSWVRQAPGKGLEWIGIINSDSNTYYASWAKGRFTISKASTTVDLKMTSLTTEDTATYFCARGVGGYSSGPDYLDIWGPGTLVTVSS

SEQ ID NO: 138 - protein, 113 aa, Rattus rattus
ADIVMTQTPASVEAAVGGTVTIKCQASQSIYRSLAWYQQKPGQPPKLLMYHASDLASGVPSRFKGSGSGTEYTLTISDLECADAATYYCQSYYYSGINTYGNAFGGGTEVVVK

SEQ ID NO: 139 - DNA, 357 bp, Rattus rattus
cagtcggtggaggagtccgggggtcgcctggtcacgcctgggacacccctgacactcgcctgcacagtctctggaatcgacctcagtacctggacaatgagttgggtccgccaggctccagggaaggggctggaatggatcggaatcattaatagtgatagtaatacatattacgcgagctgggcaaaaggccgattcaccatctccaaagcctcgaccacggtggatctgaaaatgaccagtctgacaaccgaggacacggccacctatttctgtgccagaggtgttggtggttatagtagtggtcctgactatttggacatctggggcccaggcaccctggtcaccgtctcctca

SEQ ID NO: 140 - DNA, 339 bp, Rattus rattus
gctgacattgtgatgacccagactccagcctccgtggaggcagctgtgggaggcacagtcaccatcaagtgccaggccagtcagagcatttacaggtctttagcctggtatcagcagaaaccagggcagcctcccaagctcctgatgtatcatgcatccgatctggcctctggggtcccatcgcggttcaaaggcagtggatctgggacagagtacactctcaccatcagcgacctggagtgtgccgatgctgccacttattattgtcaaagctattattatagtggtattaatacttatgggaatgcttttggcggagggaccgaggtggtggtcaaa

SEQ ID NO: 141 - protein, 5 aa, Rattus rattus
TNGMG

SEQ ID NO: 142 - protein, 16 aa, Rattus rattus
TIRVTAGTDYANWAKG

SEQ ID NO: 143 - protein, 13 aa, Rattus rattus
GHANNDAYVYFNL

SEQ ID NO: 144 - protein, 11 aa, Rattus rattus
QASQSIDRYLA

SEQ ID NO: 145 - protein, 7 aa, Rattus rattus
YTSKLAS

SEQ ID NO: 146 - protein, 11 aa, Rattus rattus
QSYFTSDDANS

SEQ ID NO: 147 - protein, 119 aa, Rattus rattus
QSLEESGGRLVTPGGSLTLTCTVSGIDLSTNGMGWVRQGPGKGLEWIGTIRVTAGTDYANWAKGRFTISKTSSTTVDLKMTSLTTEDTATYFCARGHANNDAYVYFNLWGQGTLVTVSS

SEQ ID NO: 148 - protein, 109 aa, Rattus rattus
AFELTQTPASVEAGVGGTVTIKCQASQSIDRYLAWYQQKSGQPPKLLISYTSKLASGVPSRFKGSGSGTEFTLTISDLECADAATYYCQSYFTSDDANSFGGGTEVVVK

SEQ ID NO: 149 - DNA, 357 bp, Rattus rattus
cagtcactggaggagtccgggggtcgcctggtaacgcctggaggatccctgacactcacctgcacagtctctggaatcgacctcagtactaatggaatgggctgggtccggcagggtccagggaaggggctggaatggatcggaaccattcgtgtaactgctggtacggactacgcgaactgggcgaaaggccgattcaccatctccaaaacctcgtcgaccacggtggatctgaaaatgaccagtctgacaaccgaggacacggccacctatttctgtgccagagggcatgctaataatgatgcctatgtttattttaatttgtggggccaaggcaccctggtcaccgtctcctca

SEQ ID NO: 150 - DNA, 327 bp, Rattus rattus
gcattcgaattgacccagactccagcctccgtggaggcaggtgtgggaggcacagtcaccatcaagtgccaggccagtcagagcattgacaggtacttagcctggtatcagcagaaatcagggcagcctcccaagctcctgatcagttatacatccaaactggcatctggggtcccatcgcggttcaaaggcagtggatctgggacagaattcactctcaccatcagcgacctggagtgtgccgatgctgccacttactactgtcaaagctattttactagtgatgatgctaatagtttcggcggagggaccgaggtggtggtcaaa

SEQ ID NO: 151 - protein, 5 aa, Rattus rattus
TDGMG

SEQ ID NO: 152 - protein, 16 aa, Rattus rattus
TIRVTGGTDFATWAKG

SEQ ID NO: 153 - protein, 13 aa, Rattus rattus
GHANNDAYVYFNL

SEQ ID NO: 154 - protein, 11 aa, Rattus rattus
QASQSIDRYLA

SEQ ID NO: 155 - protein, 7 aa, Rattus rattus
AAANLAS

SEQ ID NO: 156 - protein, 11 aa, Rattus rattus
QSYFTSDDGNT

SEQ ID NO: 157 - protein, 119 aa, Rattus rattus
QSLAESGGRLVTPGGSLTLTCTVSGIDLSTDGMGWVRQGPGKGLEWIGTIRVTGGTDFATWAKGRFTIAKTSSTTVDLKMTSLTTEDTATYFCARGHANNDAYVYFNLWGQGTLVTVSS

SEQ ID NO: 158 - protein, 109 aa, Rattus rattus
AFELTQTPASVEAGVGGTVTIKCQASQSIDRYLAWYQQKPGQPPKLLMAAAANLASGVPSRFKGSRSGTEFTLTISDLECADGATYYCQSYFTSDDGNTFGGGTEVVVK

SEQ ID NO: 159 - DNA, 357 bp, Rattus rattus
cagtcgctggcggagtccgggggtcgcctggtaacgcctggaggatccctgacactcacctgcacagtctctggaatcgacctcagtactgatggaatgggctgggtccggcagggtccagggaaggggctggaatggatcggaaccattcgtgtgactggtggtacggacttcgcgacctgggcgaaaggccgatttaccatcgccaaaacctcgtcgaccacggtggatctgaaaatgaccagtctgacaaccgaggacacggccacctatttctgtgccagaggacatgctaataatgatgcctatgtttattttaatttgtggggccaaggcaccctggtcaccgtctcctca

SEQ ID NO: 160 - DNA, 327 bp, Rattus rattus
gcattcgaattgacccagactccagcctccgtggaggcaggtgtgggaggcacagtcaccatcaagtgccaggccagtcagagcattgacaggtacttagcctggtatcagcagaaaccagggcagcctcccaagctcctgatggctgctgcagccaatctggcatctggggtcccatcgcggttcaaaggcagtagatctgggacagagttcactctcaccatcagcgacctggagtgtgccgatggtgccacttactactgtcaaagctattttactagtgatgatggtaatactttcggcggagggaccgaggtggtggtcaaa

SEQ ID NO: 161 - protein, 5 aa, Rattus rattus
RYRMS

SEQ ID NO: 162 - protein, 16 aa, Rattus rattus
TINNDAGASYASWVNG

SEQ ID NO: 163 - protein, 12 aa, Rattus rattus
AGDNIYVDLYNL

SEQ ID NO: 164 - protein, 11 aa, Rattus rattus
QASENIFSYLA

SEQ ID NO: 165 - protein, 7 aa, Rattus rattus
GASNLAS

SEQ ID NO: 166 - protein, 13 aa, Rattus rattus
QFTNYGSSDIRSP

SEQ ID NO: 167 - protein, 118 aa, Rattus rattus
QSLEESGGRLVTPGTPLTLTCTASGFTMNRYRMSWVRQAPGKGLEWIGTINNDAGASYASWVNGRFTISKTSSTTVDLKITGPTTEDTATYFCARAGDNIYVDLYNLWGPGTLVTVSS

SEQ ID NO: 168 - protein, 111 aa, Rattus rattus
DVVMTQTPASVSGAVGGTVTIKCQASENIFSYLAWYQQKPGQPPKLLFYGASNLASGVPSRFSGSRSGTEFTLTISDLECADAATYYCQFTNYGSSDIRSPFGGGTEVVVK

SEQ ID NO: 169 - DNA, 354 bp, Rattus rattus
cagtcgctggaggagtccgggggtcgcctggtcacgcctgggacacccctgacactcacctgcacagcctctggattcaccatgaatagatatagaatgagttgggtccgccaggctccagggaaggggctggaatggatcggaaccattaataatgacgctggcgcatcctacgcgagttgggtgaatggccgatttaccatctccaaaacctcgtcgaccacggtggatctgaaaatcaccggcccgacaaccgaggacacggccacgtatttctgtgccagagctggtgataatatttatgttgatctatataatttgtggggcccaggcaccctggtcaccgtctcctca

SEQ ID NO: 170 - DNA, 333 bp, Rattus rattus
gatgttgtgatgacccagactccagcctccgtgtctggagctgtgggaggcacagtcaccatcaagtgccaggccagtgagaacattttcagctacttagcctggtatcagcagaaaccagggcagcctcccaagctccttttctatggtgcatccaacctggcatctggggtcccatcgcggttcagcggcagtagatctgggacagagttcactctcaccatcagcgacctggagtgtgccgatgctgccacttactactgtcaatttactaattatggtagtagtgatattcgtagtcctttcggcggcgggaccgaggtggtggtcaaa

SEQ ID NO: 171 - protein, 5 aa, Rattus rattus
TNGMG

SEQ ID NO: 172 - protein, 16 aa, Rattus rattus
TIRVTGGTDYATWAKG

SEQ ID NO: 173 - protein, 13 aa, Rattus rattus
GHANNDAYVYFNL

SEQ ID NO: 174 - protein, 11 aa, Rattus rattus
QASQSIDRYLA

SEQ ID NO: 175 - protein, 7 aa, Rattus rattus
YTSTLTS

SEQ ID NO: 176 - protein, 11 aa, Rattus rattus
QSYFTSDDANT

SEQ ID NO: 177 - protein, 119 aa, Rattus rattus
QSLEESGGRLVTPGGSLTLTCTVSGIDLSTNGMGWVRQGPGKGLEWIGTIRVTGGTDYATWAKGRFTISKTSSTTVDLKMTSPTTEDTATYFCARGHANNDAYVYFNLWGQGTLVTVSS

SEQ ID NO: 178 - protein, 109 aa, Rattus rattus
AFELTQTPASVEAGVGGTVTIKCQASQSIDRYLAWYQQKPGQPPKLLISYTSTLTSGVPSRFKGSGSGTEFTLTISDLECADAATYYCQSYFTSDDANTFGGGTEVVVK

SEQ ID NO: 179 - DNA, 357 bp, Rattus rattus
cagtcgctggaggagtccgggggtcgcctggtaacgcctggaggatccctgacactcacctgtacagtctctggaatcgacctcagtactaatggaatgggctgggtccggcagggtccagggaaggggctggaatggatcggaaccattcgtgtaactggtggtacggactacgcgacctgggcgaaaggccgattcaccatctccaaaacctcgtcgaccacggtggatctgaaaatgaccagcccgacaaccgaggacacggccacctatttctgtgccagagggcatgctaataatgatgcctatgtttattttaatttgtggggccaaggcaccctggtcaccgtctcctca

SEQ ID NO: 180 - DNA, 327 bp, Rattus rattus
gcattcgaattgacccagactccagcctccgtggaggcaggtgtgggaggcacagtcaccatcaagtgccaggccagtcagagcattgacaggtacttagcctggtatcagcagaaaccagggcagcctcccaagctcctgatcagttatacatccactctgacatctggggtcccatcgcggttcaaaggcagtggatctgggacagagttcactctcaccatcagcgacctggagtgtgccgatgctgccacttactactgtcaaagctattttactagtgatgatgctaatactttcggcggagggaccgaggtggtggtcaaa

SEQ ID NO: 181 - protein, 5 aa, Rattus rattus
KYWMA

SEQ ID NO: 182 - protein, 16 aa, Rattus rattus
ILSTTGSTYYASWAKG

SEQ ID NO: 183 - protein, 12 aa, Rattus rattus
GNYIGNGYTWDI

SEQ ID NO: 184 - protein, 11 aa, Rattus rattus
QASQSIGSYLA

SEQ ID NO: 185 - protein, 7 aa, Rattus rattus
SPSTLAS

SEQ ID NO: 186 - protein, 8 aa, Rattus rattus
QSTAVGIT

SEQ ID NO: 187 - protein, 117 aa, Rattus rattus
QSVEESGGRLVTPGTPLTLTCTVSGIDFSKYWMAWVRQAPGEGLEYIGILSTTGSTYYASWAKGRFTISKTSTTVDLKITSLTTEDTATYFCARGNYIGNGYTWDIWGPGTLVTVSS

SEQ ID NO: 188 - protein, 106 aa, Rattus rattus
DPVLTQTPSSASEPVGGTVTIKCQASQSIGSYLAWYQQKPGQPPKLLIYSPSTLASGVSSRFKGSRSGTEFTLTISDLECADAATYYCQSTAVGITFGGGTEVVVK

SEQ ID NO: 189 - DNA, 351 bp, Rattus rattus
cagtcggtggaggagtccgggggtcgcctggtcacgcctgggacacccctgacactcacctgcacagtctctggaatcgactttagtaaatattggatggcctgggtccgccaggctccaggggaggggctggaatacatcggaatccttagtactactggtagcacatactacgcgagctgggcgaaaggccgattcaccatctccaaaacctcgaccacggtggatctgaaaatcaccagtctgacaaccgaggacacggccacatatttctgtgccagagggaattatattggtaatggttatacttgggacatctggggcccaggcaccctggtcaccgtctcctca

SEQ ID NO: 190 - DNA, 318 bp, Rattus rattus
gaccctgtgctgacccagactccatcttccgcgtctgaacctgtgggaggcacagtcaccatcaagtgccaggccagtcagagcattggtagttacttagcctggtatcagcagaaaccagggcagcctcccaagctcctgatctattctccatccactctggcatctggggtctcatcgcggttcaaaggcagtagatctgggacagaattcactctcaccatcagcgacctggagtgtgccgatgctgccacttactactgtcaatctactgctgttggtattactttcggcggagggaccgaggtggtggtcaaa

SEQ ID NO: 191 - protein, 5 aa, Rattus rattus
SYYII

SEQ ID NO: 192 - protein, 16 aa, Rattus rattus
IIDTGGSADYASWAKG

SEQ ID NO: 193 - protein, 9 aa, Rattus rattus
ADVGSVGSI

SEQ ID NO: 194 - protein, 11 aa, Rattus rattus
QASQNIYTNLA

SEQ ID NO: 195 - protein, 7 aa, Rattus rattus
YASTLAS

SEQ ID NO: 196 - protein, 12 aa, Rattus rattus
QCTYIKWRHVCG

SEQ ID NO: 197 - protein, 114 aa, Rattus rattus
QSVEESGGRLVTPGTPLTLTCTASGFTITSYYIIWVRQAPGEGLEYIGIIDTGGSADYASWAKGRFTISRTSTTVDLKMTSLTTEDTATYFCARADVGSVGSIWGPGTLVTVSS

SEQ ID NO: 198 - protein, 110 aa, Rattus rattus
DVAMTQTPSSVSAAVGGTVTIECQASQNIYTNLAWYQQKPGQRPKLLIYYASTLASGVPSRFSGSGSGTEFSLTISDLECADAATYYCQCTYIKWRHVCGFGGGTEVVVK

SEQ ID NO: 199 - DNA, 342 bp, Rattus rattus
cagtcggtggaggagtccgggggtcgcctggtcacgcctgggacacccctgacactcacctgcacagcctctggattcaccatcactagttactacataatctgggtccgccaggctccaggggaggggctggaatacatcggaatcattgatactggtggtagcgcggactacgccagctgggcgaaaggccgattcaccatctccagaacctcgaccacggtggatctgaaaatgaccagtctgacaaccgaggacacggccacttatttctgtgccagggcagatgttggtagtgttggttcgatctggggcccaggcaccctggtcaccgtctcctca

SEQ ID NO: 200 - DNA, 330 bp, Rattus rattus
gatgttgcgatgacccagactccatcctccgtgtctgcagctgtgggaggcacagtcaccatcgagtgccaggccagtcagaacatttacaccaatttagcctggtatcagcagaaaccagggcagcgtcccaagctcctgatctattatgcatccactctggcatctggggtcccatcgcggttcagcggcagtggatctgggacagagttcagtctcaccatcagcgacctggagtgtgccgatgctgccacttactactgtcaatgtacttatataaagtggaggcatgtttgtggtttcggcggagggaccgaggtggtggtcaaa

SEQ ID NO: 201 - protein, 5 aa, Rattus rattus
NYYMG

SEQ ID NO: 202 - protein, 16 aa, Rattus rattus
WITSGRIAAYANWAKG

SEQ ID NO: 203 - protein, 14 aa, Rattus rattus
TTTSDAYFFPRLGL

SEQ ID NO: 204 - protein, 12 aa, Rattus rattus
QSSQSVDNNRLS

SEQ ID NO: 205 - protein, 7 aa, Rattus rattus
DASTLAS

SEQ ID NO: 206 - protein, 11 aa, Rattus rattus
AGGYSFPSENA

SEQ ID NO: 207 - protein, 119 aa, Rattus rattus
QSVEESGGRLVTPGTPLTLTCTVSGFSLNNYYMGWVRQAPGKGLEYIGWITSGRIAAYANWAKGRFTISKTSTTVDLKITSPTIGDTATHFCATTTTSDAYFFPRLGLWGQGTLVTVSS

SEQ ID NO: 208 - protein, 109 aa, Rattus rattus
AVLTQTPSPVSAAVEGTVTINCQSSQSVDNNRLSWFQQKPGQPPKLLIYDASTLASGVPPRFKGSGSGTQFTLTISDVQCDDAATYYCAGGYSFPSENAFGGGTEVVVK

SEQ ID NO: 209 - DNA, 357 bp, Rattus rattus
cagtcggtggaggagtccgggggtcgcctggtcacgcctgggacacccctgacactcacctgcacagtctctggattctccctcaataactactacatgggctgggtccgccaggctccagggaaggggctggaatacatcggatggattacttctggtcgtatcgcagcctacgcgaactgggcaaaaggccgattcaccatctctaaaacctcgaccacggtggatctcaaaatcaccagtccgacaatcggggacacggccacccatttctgtgccacaactactactagtgatgcctatttttttcctcggttgggtctctggggccagggcaccctggtcaccgtctcctca

SEQ ID NO: 210 - DNA, 327 bp, Rattus rattus
gccgtgctgacccagactccatctcccgtgtctgcagctgtggaaggcacagtcaccatcaattgccagtccagtcagagtgttgataacaaccgcttatcctggtttcagcagaaaccagggcagcctcccaagctcctgatctacgatgcatccactctggcatctggggtcccaccgcgattcaaaggcagtggatctgggacacagttcactctcaccatcagcgacgtgcagtgtgacgatgctgccacttattactgtgcaggcggttatagttttcctagtgagaatgctttcggcggagggaccgaggtggtggtcaaa

SEQ ID NO: 211 - protein, 5 aa, Rattus rattus
DYYMT

SEQ ID NO: 212 - protein, 16 aa, Rattus rattus
IISSSGNTYNATWANG

SEQ ID NO: 213 - protein, 13 aa, Rattus rattus
EDGSGGRFDAFDL

SEQ ID NO: 214 - protein, 11 aa, Rattus rattus
QASQSISSYCS

SEQ ID NO: 215 - protein, 7 aa, Rattus rattus
RASILQS

SEQ ID NO: 216 - protein, 12 aa, Rattus rattus
QQGSTIANIENT

SEQ ID NO: 217 - protein, 118 aa, Rattus rattus
QSVEESGGRLVTPGTPLTLTCTASGFSLSDYYMTWVRQAPGKGLEWIGIISSSGNTYNATWANGRFTISKTSTTVDLKITSPTTEDTATYFCAREDGSGGRFDAFDLWGPGTLVTVSS

SEQ ID NO: 218 - protein, 110 aa, Rattus rattus
  misc_feature (105)..(106): Xaa can be any naturally occurring amino acid
AYDMTQTPASVEAAVGGTVTIKCQASQSISSYCSWYQQKPGQPPKLLIYRASILQSGVPSRFKGSGSGTDFTLTISDLECADAATYYCQQGSTIANIENTFGGGXXVVVK

SEQ ID NO: 219 - DNA, 354 bp, Rattus rattus
cagtcggtggaggagtccgggggtcgcctggtcacgcctgggacacccctgacactcacctgcacagcctctggattctccctcagtgactactacatgacctgggtccgccaggctccagggaaggggctggaatggatcggaatcattagtagtagtggtaacacatacaacgcgacctgggcgaacggccgattcaccatctccaaaacctcgaccacggtggatctgaaaatcaccagtccgacaaccgaggacacggccacctacttctgtgccagagaagatggtagtggtggtaggtttgatgcttttgatctctggggcccaggcaccctggtcaccgtctcctca

SEQ ID NO: 220 - DNA, 330 bp, Rattus rattus
  misc_feature (315)..(316): n is a, c, g, or t
  misc_feature (318)..(318): n is a, c, g, or t
gcctatgatatgacccagactccagcctctgtggaggcagctgtgggaggcacagtcaccatcaagtgccaggccagtcagagcattagtagttactgctcctggtatcagcagaaaccagggcagcctcccaagctcctgatctacagggcatccattctgcaatctggggtcccatcgcggtttaaaggcagtggatctgggacagatttcacgctcaccatcagcgacctggagtgtgccgatgctgccacttactactgtcaacagggtagtactattgctaatattgagaatactttcggcggagggacnnangtggtggtcaaa

SEQ ID NO: 221 - protein, 5 aa, Rattus rattus
RYAMG

SEQ ID NO: 222 - protein, 17 aa, Rattus rattus
IITSNSGDTYYATWAKG

SEQ ID NO: 223 - protein, 8 aa, Rattus rattus
GYIGAPNL

SEQ ID NO: 224 - protein, 13 aa, Rattus rattus
QSSKTVYYNNYLS

SEQ ID NO: 225 - protein, 7 aa, Rattus rattus
YASTLSS

SEQ ID NO: 226 - protein, 11 aa, Rattus rattus
AGGYNSDSDNT

SEQ ID NO: 227 - protein, 114 aa, Rattus rattus
QSLEESGGRLVTPGTPLTLTCTVSGIDLSRYAMGWVRQAPGKGLEFIGIITSNSGDTYYATWAKGRFTISKTSTTVDLKITSPTTEDTATYFCVRGYIGAPNLWGPGTLVTVSS

SEQ ID NO: 228 - protein, 110 aa, Rattus rattus
AVLTQTPSPVSAAVGGTVSISCQSSKTVYYNNYLSWFQQKPGQPPKELIYYASTLSSGVPSRFKGSGSGTQFTLTISDVQCDDAATYYCAGGYNSDSDNTFGGGTEVVVK

SEQ ID NO: 229 - DNA, 342 bp, Rattus rattus
cagtcgctggaggagtccgggggtcgcctggtcacgcctgggacacccctgacactcacctgcacagtctctggaatcgacctcagtaggtatgcaatgggctgggtccgccaggctccagggaaggggctggaattcatcggaatcattactagtaatagtggtgatacatactacgcgacctgggcgaaaggccgattcaccatctccaaaacctcgaccacggtggatctgaaaatcaccagtccgacaaccgaagacacggccacctatttctgtgtcagaggctatattggtgctcctaatttatggggcccaggcaccctggtcaccgtctcctca

SEQ ID NO: 230 - DNA, 330 bp, Rattus rattus
gccgtgctgacccagactccatctcccgtgtctgcagctgtgggaggcacagtcagcatcagttgccagtccagtaagactgtttattataataattacttatcctggtttcagcagaaaccagggcagcctcccaaggaactgatctattatgcatccactctgtcatctggggtcccatcgcggttcaaaggcagtggatctgggacacaattcactctcaccatcagcgacgtgcagtgtgacgatgctgccacttattactgtgcaggcggttataatagtgatagtgataatactttcggcggggggaccgaggtggtggtcaaa

SEQ ID NO: 231 - protein, 5 aa, Rattus rattus
GYVMG

SEQ ID NO: 232 - protein, 16 aa, Rattus rattus
WISFGGDPYYASWVKG

SEQ ID NO: 233 - protein, 6 aa, Rattus rattus
STYGDI

SEQ ID NO: 234 - protein, 11 aa, Rattus rattus
QASQSISSRLA

SEQ ID NO: 235 - protein, 7 aa, Rattus rattus
SASTLAS

SEQ ID NO: 236 - protein, 14 aa, Rattus rattus
QNHGYATSESYTNT

SEQ ID NO: 237 - protein, 111 aa, Rattus rattus
QSVEESGGRLVTPGTPLTLTCTASGFSLIGYVMGWFRQAPGKGLEYIGWISFGGDPYYASWVKGRFTISKTSTTVDLKMTSLTTEDTATYFCARSTYGDIWGPGTLVTVSS

SEQ ID NO: 238 - protein, 113 aa, Rattus rattus
ADIVMTQTPASVEAAVGGTVTIKCQASQSISSRLAWYQLKPGQPPKLLIYSASTLASGVPSRFKGSGSGTEFTLTISDLECADAATYYCQNHGYATSESYTNTFGGGTEVVVK

SEQ ID NO: 239 - DNA, 333 bp, Rattus rattus
cagtcggtggaggagtccgggggtcgcctggtcacgcctgggacacccctgacactcacctgcacagcctctggattctccctcattggctatgtaatgggctggttccgccaggctccagggaaggggctggaatatatcggatggattagttttggtggtgatccatactacgcgagctgggtgaaaggccgattcaccatctccaaaacctcgaccacggtggatctgaaaatgaccagtctgacaaccgaggacacggccacctatttctgtgccagatctacttatggtgacatctggggcccaggcaccctggtcaccgtctcctca

SEQ ID NO: 240 - DNA, 339 bp, Rattus rattus
gctgacattgtgatgacccagactccagcctccgtggaggcagctgtgggaggcacagtcaccatcaagtgccaggccagtcagagcattagtagtagattagcctggtatcagctgaaaccagggcagcctcccaaactcctgatctattctgcatccactctggcatctggggtcccatcgcggttcaaaggcagtggatctgggacagagttcactctcaccatcagcgacctggagtgtgccgatgctgccacttactactgtcaaaaccatggttatgctactagtgagagttatactaatactttcggcggagggaccgaggtggtggtcaaa

SEQ ID NO: 241 - protein, 5 aa, Rattus rattus
DHTMI

SEQ ID NO: 242 - protein, 16 aa, Rattus rattus
FINIGGRAYYASWAKG

SEQ ID NO: 243 - protein, 11 aa, Rattus rattus
GDTHGYSYPEI

SEQ ID NO: 244 - protein, 11 aa, Rattus rattus
QASQTIYSYLS

SEQ ID NO: 245 - protein, 7 aa, Rattus rattus
RASTLAS

SEQ ID NO: 246 - protein, 11 aa, Rattus rattus
QNYYDSTSSNA

SEQ ID NO: 247 - protein, 116 aa, Rattus rattus
QSVEESGGRLVTPGTPLTLTCTVSGFSFYDHTMIWVRQAPGKGLEYIGFINIGGRAYYASWAKGRFTISRTSTTVDLEMTSLTTEDTATYFCARGDTHGYSYPEIWGPGTLVTVSS

SEQ ID NO: 248 - protein, 110 aa, Rattus rattus
ADIVMTQTPASVEAGVGGTVTIKCQASQTIYSYLSWYQQKPGQPPKLLIYRASTLASGVPSRFRGSGSGTEFTLTISDLECADAAAYYCQNYYDSTSSNAFGKGTEVVVK

SEQ ID NO: 249 - DNA, 348 bp, Rattus rattus
cagtcggtggaggagtccgggggtcgcctggtcacgcctgggacacccctgacactcacctgcacagtctctggattctccttctatgatcatacaatgatctgggtccgccaggctccaggaaaggggctggaatacatcggattcataaatattggtggtcgcgcatactacgcgagctgggcaaaaggccgattcaccatctccagaacctcgaccacggtggatctggaaatgaccagtctgacaaccgaggacacggccacctatttctgtgccagaggggatactcatggttattcttatcccgagatctggggcccaggcaccctggtcaccgtctcctca

SEQ ID NO: 250 - DNA, 330 bp, Rattus rattus
gctgacattgtgatgacccagactccagcctccgtggaggcaggtgtgggaggcacagtcaccatcaagtgccaggccagtcagaccatttacagctacttatcctggtatcagcagaaaccagggcagcctcccaaactcctgatctacagggcatccactctggcatctggggtcccatcgcggttcagaggcagtggatctgggacagagttcactctcaccatcagcgacctggagtgtgccgatgctgccgcttattattgtcaaaactattatgatagtactagttctaatgctttcggcaaagggaccgaggtggtggtcaaa

SEQ ID NO: 251 - protein, 5 aa, Rattus rattus
SYAMN

SEQ ID NO: 252 - protein, 16 aa, Rattus rattus
MIATGGKTFYAGWAEG

SEQ ID NO: 253 - protein, 10 aa, Rattus rattus
GYVGDIYFGF

SEQ ID NO: 254 - protein, 13 aa, Rattus rattus
QSSKSVYYNTHLS

SEQ ID NO: 255 - protein, 7 aa, Rattus rattus
RASTLAS

SEQ ID NO: 256 - protein, 11 aa, Rattus rattus
AGGYANDVDNG

SEQ ID NO: 257 - protein, 115 aa, Rattus rattus
QSVEESGGRLVTPGTPLTLTCTVSGFSLSSYAMNWVRQAPGKGLEYTGMIATGGKTFYAGWAEGRFTISKTSTTVDLHIASLTTEDTATYYCARGYVGDIYFGFWGPGTLVTVSS

SEQ ID NO: 258 - protein, 110 aa, Rattus rattus
AVLTQTPSPVSAGVGGTVTISCQSSKSVYYNTHLSWFQQKPGQPPKLLIYRASTLASGVPSRFKGSGSGTQFTLTISDVQCDDAATYYCAGGYANDVDNGFGGGTEVVVK

SEQ ID NO: 259 - DNA, 345 bp, Rattus rattus
cagtcggtggaggagtccgggggtcgcctggtcacgcctgggacacccctgacactcacctgcacagtctctggattttccctcagtagctatgcaatgaattgggtccgccaggctccagggaaggggctggaatacaccggaatgattgctactggtggtaagacattttacgcgggctgggcggaaggccgattcaccatctccaaaacctcgaccacggtggatctgcatatcgccagtctgacaaccgaggacacggccacttattactgtgccagaggttatgttggtgatatatattttggtttctggggcccaggcaccctggtcaccgtctcctca

SEQ ID NO: 260 - DNA, 330 bp, Rattus rattus
gccgtgctgacccagactccatctcccgtgtctgcaggtgtgggaggcacagtcaccatcagttgccagtccagtaagagtgtttattataatacgcacttgtcctggtttcagcagaaaccagggcagcctcccaagctcctgatctatagggcttccactctggcatctggggtcccatcgcggttcaaaggcagtggatctgggacacagttcactctcaccatcagcgacgtacagtgtgacgatgctgccacttactactgtgcaggcggttatgctaatgatgttgataatggtttcggcggagggaccgaggtggtggtcaaa

SEQ ID NO: 261 - protein, 5 aa, Rattus rattus
SYAMS

SEQ ID NO: 262 - protein, 16 aa, Rattus rattus
VMNTGGSAHYATWAKG

SEQ ID NO: 263 - protein, 8 aa, Rattus rattus
GYIGSPNL

SEQ ID NO: 264 - protein, 13 aa, Rattus rattus
QSSKSVYYNNYLS

SEQ ID NO: 265 - protein, 7 aa, Rattus rattus
LASTLAS

SEQ ID NO: 266 - protein, 10 aa, Rattus rattus
AGGYTDSDDA

SEQ ID NO: 267 - protein, 113 aa, Rattus rattus
QSVEESGGRLVTPGTPLTLTCTVSGFSLSSYAMSWVRQAPGKGLEYIGVMNTGGSAHYATWAKGRFTISKTSTTVDLKITSPTTADTATYFCARGYIGSPNLWGPGTLVTVSS

SEQ ID NO: 268 - protein, 109 aa, Rattus rattus
AVLTQTPSPVSAAVGGTVSISCQSSKSVYYNNYLSWFQQKPGQPPKQLIYLASTLASGVPSRFKGSGSGTEFTLTISDVQCADAATYYCAGGYTDSDDAFGGGTEVVVK

SEQ ID NO: 269 - DNA, 339 bp, Rattus rattus
cagtcggtggaggagtccgggggtcgcctggtcacgcctgggacacccctgacactcacctgcaccgtctctggattctccctcagtagctatgcaatgagctgggtccgccaggctccagggaaggggctggaatacatcggagtcatgaatactggtggtagcgcacactacgcgacctgggcgaaaggccgattcaccatctccaaaacctcgaccacggtggatctgaaaatcaccagtccgacaaccgcggacacggccacctatttctgtgccagaggctatattggttctcctaacttgtggggcccaggcaccctggtcaccgtctcctca

SEQ ID NO: 270 - DNA, 327 bp, Rattus rattus
gccgtgctgacccagactccatctcccgtgtctgcagctgtgggaggcacagtcagcatcagttgccaatccagtaagagtgtttattataacaactacttatcctggtttcagcagaaaccagggcagcctcccaagcaactgatctatctggcatctactctggcatctggggtcccatcgcggttcaaaggcagtggatctgggacagagttcactctcaccatcagcgacgtgcagtgtgccgatgctgccacttactactgtgcaggcggttatacagatagtgatgatgctttcggcggagggaccgaggtggtggtcaaa

SEQ ID NO: 271 - protein, 5 aa, Rattus rattus
SYAMN

SEQ ID NO: 272 - protein, 16 aa, Rattus rattus
MIATGGKTFYAGWAEG

SEQ ID NO: 273 - protein, 10 aa, Rattus rattus
GYVGDIYFGF

SEQ ID NO: 274 - protein, 13 aa, Rattus rattus
QSSKSVYYNTHLP

SEQ ID NO: 275 - protein, 7 aa, Rattus rattus
RASTLAS

SEQ ID NO: 276 - protein, 11 aa, Rattus rattus
AGGYANDVDNG

SEQ ID NO: 277 - protein, 115 aa, Rattus rattus
QSVEESGGRLVTPGTPLTLTCTVSGFSLSSYAMNWVRQAPGKGLEYIGMIATGGKTFYAGWAEGRFTISKTSTTVDLHIASLTTEDTATYYCARGYVGDIYFGFWGPGTLVTVSS

SEQ ID NO: 278 - protein, 110 aa, Rattus rattus
AVLTQTPSPVSAGVGGTVTISCQSSKSVYYNTHLPWFQQKPGQPPKLLIYRASTLASGVPSRFKGSGSGTQFTLTISDVQCDDAATYYCAGGYANDVDNGFGGGTEVVVK

SEQ ID NO: 279 - DNA, 345 bp, Rattus rattus
cagtcggtggaggagtccgggggtcgcctggtcacgcctgggacacccctgacactcacctgcacagtctctggattttccctcagtagctatgcaatgaattgggtccgccaggctccagggaaggggctggaatacatcggaatgattgctactggtggtaagacattttacgcgggctgggcggaaggccgattcaccatctccaaaacctcgaccacggtggatctgcatatcgccagtctgacaaccgaggacacggccacttattactgtgccagaggttatgttggtgatatatattttggtttctggggcccaggcaccctggtcaccgtctcctca

SEQ ID NO: 280 - DNA, 330 bp, Rattus rattus
gccgtgctgacccagactccatctcccgtgtctgcaggtgtgggaggcacagtcaccatcagttgccagtccagtaagagtgtttattataatacgcacttgccctggtttcagcagaaaccagggcagcctcccaagctcctgatctatagggcttccactctggcatctggggtcccatcgcggttcaaaggcagtggatctgggacacagttcactctcaccatcagcgacgtacagtgtgacgatgctgccacttactactgtgcaggcggttatgctaatgatgttgataatggtttcggcggagggaccgaggtggtggtcaaa

SEQ ID NO: 281 - protein, 5 aa, Rattus rattus
AYAIH

SEQ ID NO: 282 - protein, 16 aa, Rattus rattus
IIDAGGNIVYATWALG

SEQ ID NO: 283 - protein, 12 aa, Rattus rattus
GLSSYSNSAMDL

SEQ ID NO: 284 - protein, 11 aa, Rattus rattus
QASQNIYTYLA

SEQ ID NO: 285 - protein, 7 aa, Rattus rattus
QASTLAS

SEQ ID NO: 286 - protein, 10 aa, Rattus rattus
QSTAGNAYNA

SEQ ID NO: 287 - protein, 117 aa, Rattus rattus
QSVEESGGRLVTPGTPLTLTCTVSGFSLSAYAIHWVRQAPGKGLEYIGIIDAGGNIVYATWALGRFTISKTSTTVDLKITSPTTEDTATYFCGRGLSSYSNSAMDLWGPGTLVTVSS

SEQ ID NO: 288 - protein, 108 aa, Rattus rattus
DVVMTQTPASVSEPVGGTVTMKCQASQNIYTYLAWYQQKPGRPPKLLIYQASTLASGVSSRFSGSGSGTEFTLTISDLECADAATYYCQSTAGNAYNAFGGGTEVVVK

SEQ ID NO: 289 - DNA, 351 bp, Rattus rattus
cagtcggtggaggagtccgggggtcgcctggtcacgcctgggacacccctgacactcacctgcacagtctctggattctctctcagtgcctatgcaattcactgggtccgccaggctccagggaaggggctggaatacatcggaatcattgatgctggtggtaacatagtctacgcgacttgggcgttaggccgattcaccatctccaaaacctcgaccacggtggatctgaaaatcaccagtccgacaaccgaggacacggccacctatttctgtggcagaggactttcgagttatagtaatagtgctatggacttgtggggcccaggcaccctggtcaccgtctcctca

SEQ ID NO: 290 - DNA, 324 bp, Rattus rattus
gatgttgtgatgacccagactccagcctccgtgtctgaacctgtgggaggcacagtcaccatgaagtgccaggccagtcagaacatttacacctacttagcctggtatcagcagaaaccagggcggcctcccaagctcctgatctatcaggcatccactctggcatctggggtctcatcgcggttcagtggcagtggatctgggacagagttcactctcaccatcagcgacctggagtgtgccgatgctgccacttattattgtcaaagtactgctggaaatgcttataatgctttcggcggggggaccgaggtggtggtcaaa

SEQ ID NO: 291 - protein, 5 aa, Rattus rattus
SYAMN

SEQ ID NO: 292 - protein, 16 aa, Rattus rattus
VITSSGTIYYANWATG

SEQ ID NO: 293 - protein, 12 aa, Rattus rattus
GITGAVTTGLSL

SEQ ID NO: 294 - protein, 11 aa, Rattus rattus
QASQNIYNNLA

SEQ ID NO: 295 - protein, 7 aa, Rattus rattus
AASNLAS

SEQ ID NO: 296 - protein, 12 aa, Rattus rattus
LGGYYGNNVGNT

SEQ ID NO: 297 - protein, 119 aa, Rattus rattus
QEQLKESGGGLVTPGTALTLTCTASGSDISSYAMNWVRQAPGKGLEYIGVITSSGTIYYANWATGRFTISKASSTTVDLKTTRLTTEDTATYFCARGITGAVTTGLSLWGPGTLVTVSS

SEQ ID NO: 298 - protein, 110 aa, Rattus rattus
DVVMTQTPPSVSEPVGGTVTIKCQASQNIYNNLAWYQQTPGQPPKLLGYAASNLASGVSSRFSGSGYGTEFTLTISDLECADAATYYCLGGYYGNNVGNTFGGGTEVVVK

SEQ ID NO: 299 - DNA, 357 bp, Rattus rattus
caggagcagctgaaggagtccggaggaggcctggtaacgcctgggacagccctgacactcacctgcacagcctctggatccgacatcagtagctatgcaatgaactgggtccgccaggctccagggaaggggctggaatacatcggagtcattactagtagtggtaccatatactacgcgaactgggcgacaggccgattcaccatctccaaagcctcgtcgaccacggtggatctgaaaacgacgaggctgacaaccgaggacacggccacctatttctgtgccagagggatcactggtgctgttactactggtctgagtttgtggggcccaggcaccctggtcaccgtctcctca

SEQ ID NO: 300 - DNA, 330 bp, Rattus rattus
gatgttgtgatgacccagactcctccctccgtatctgaacctgtgggaggcacagtcaccatcaagtgccaggccagtcagaacatttacaacaatttagcctggtatcagcagacaccagggcagcctcccaagctcctgggctatgctgcatccaatctggcatctggggtctcatcgcggttcagcggcagtggatatgggacagagttcactctcaccatcagcgacctggagtgtgccgatgctgccacttactactgtctaggcggttattatggtaataatgttgggaatactttcggcggagggaccgaggtggtggtcaaa

SEQ ID NO: 301 - protein, 5 aa, Rattus rattus
SYTMA

SEQ ID NO: 302 - protein, 16 aa, Rattus rattus
TIDSSGSADYASWAQS

SEQ ID NO: 303 - protein, 12 aa, Rattus rattus
PTNYEYTWALNI

SEQ ID NO: 304 - protein, 11 aa, Rattus rattus
QASQTIYSYLA

SEQ ID NO: 305 - protein, 7 aa, Rattus rattus
GASKLAS

SEQ ID NO: 306 - protein, 13 aa, Rattus rattus
QCTGYDSAYVYND

SEQ ID NO: 307 - protein, 119 aa, Rattus rattus
QSVKESEGGLFKPTDTLTLTCTVSGFSLGSYTMAWVRQAPGNGLEYIGTIDSSGSADYASWAQSRSTITRNTNLNTVTLKMTSLTAADTATYFCARPTNYEYTWALNIWGPGTLVTVSS

SEQ ID NO: 308 - protein, 111 aa, Rattus rattus
DVVMTQTPASVSEPVGGTVTIKCQASQTIYSYLAWYQQKPGQPPKLLIYGASKLASGVPSRFSGSRSGTEFTLTISDLECADAATYYCQCTGYDSAYVYNDFGGGTEVVVK

SEQ ID NO: 309 - DNA, 357 bp, Rattus rattus
cagtcggtgaaggagtccgagggaggtctcttcaagccaacggataccctgacactcacctgcacagtttctggattctccctcggtagctatacaatggcctgggtccgccaggctccagggaacgggctggaatatatcggaaccattgatagtagtggtagcgctgactacgcgagctgggcgcaaagccgatccaccatcaccagaaacaccaacctgaacacggtgactctgaaaatgaccagtctgacagccgcggacacggccacctatttctgtgcgaggcctactaattatgagtatacttgggcgcttaacatctggggcccaggcaccctggtcaccgtctcctca

SEQ ID NO: 310 - DNA, 333 bp, Rattus rattus
gatgttgtgatgacccagactccagcctccgtgtctgaacctgtgggaggcacagtcaccatcaagtgccaggccagtcagaccatttacagctacttagcctggtatcagcagaaaccagggcagcctcccaagctcctgatctatggtgcatccaaactggcatctggggtcccatcgcggttcagcggcagtagatctgggacagagttcactctcaccatcagcgacctggagtgtgccgatgctgccacttactactgtcaatgtactggttatgatagtgcttatgtctataatgatttcggcggagggaccgaggtggtggtcaaa

SEQ ID NO: 311 - protein, 115 aa, Artificial Sequence
  Synthetic
EVQLVESGGGLIQPGGSLRLSCAVSGFSLSTYAVSWVRQAPGKGLEWVGIIDSGGGTAYASWVKGRFTISRDNSKNTLYLQMNSLRAEDTAVYYCARGVTFFGLWGQGTLVTVSS

SEQ ID NO: 312 - protein, 112 aa, Artificial Sequence
  Synthetic
DIVMTQSPSTLSASVGDRVTITCQASQSISNYLSWYQQKPGKAPKLLIYRASDLASGVPSRFSGSGSGTEFTLTISSLQPDDFATYYCQAYYYSGGGSYDYAFGGGTKVEIK

SEQ ID NO: 313 - protein, 5 aa, Artificial Sequence
  Synthetic
  MISC_FEATURE (4)..(4): X at the fourth position is M or S.
TYAXS

SEQ ID NO: 314 - protein, 16 aa, Artificial Sequence
  Synthetic
  MISC_FEATURE (4)..(14): X at the fourth, fifth and sixth positions is independently G or S; X at the seventh position is F or G; X at the eighth position is A or T; X at the ninth position is A or V; X at the twelfth position is N or S; X at the fourteenth position is A or V.
IIDXXXXXXYAXWXKG

SEQ ID NO: 315 - protein, 7 aa, Artificial Sequence
  Synthetic
  MISC_FEATURE (2)..(6): X at the second position is A or V; X at the sixth position is G or N.
GXTFFXL

SEQ ID NO: 316 - protein, 11 aa, Artificial Sequence
  Synthetic
  MISC_FEATURE (8)..(11): X at the eighth position is G, N or S; X at the ninth position is A, V or Y; X at the eleventh position is A or S.
QASQSISXXLX

SEQ ID NO: 317 - protein, 7 aa, Artificial Sequence
  Synthetic
  MISC_FEATURE (3)..(6): X at the third position is A or S; X at the fourth position is D or T; X at the sixth position is A or E.
RAXXLXS

SEQ ID NO: 318 - protein, 14 aa, Artificial Sequence
  Synthetic
  MISC_FEATURE (2)..(12): X at the second position is A or S; X at the seventh position is G, N or S; X at the eighth position is G or S; X at the twelfth position is D or S.
QXYYYSXXGSYXYA

SEQ ID NO: 319 - protein, 243 aa, Artificial Sequence
  Synthetic
EVQLVESGGGLIQPGGSLRLSCAVSGFSLSTYAVSWVRQAPGKGLEWVGIIDSGGGTAYASWVKGRFTISRDNSKNTLYLQMNSLRAEDTAVYYCARGVTFFGLWGQGTLVTVSSGGGGSGGGGSGGGGSDIVMTQSPSTLSASVGDRVTITCQASQSISNYLSWYQQKPGKAPKLLIYRASDLASGVPSRFSGSGSGTEFTLTISSLQPDDFATYYCQAYYYSGGGSYDYAFGGGTKVEIKR